SEQUENCE LISTING (24 sequences)

SEQ ID NO: 1 - DNA, 18 bp, Homo sapiens
gctgccaagaagaaggtg

SEQ ID NO: 2 - DNA, 1059 bp, Homo sapiens
tcttgagtgcctcctgtctgcctggcactgttctgggcactggaggaacaacagataaaagtctgctctcctgggagcttatattctagtaggtataaacaaggaaaaactgacaaaacagacactacacaaaatagataagaaaatcatgtagtagattagaaggtgaaaagtgctgtgaagacaaagtagcagggaaggcaaagaggaagtgttggtggtttgaggttggttgcaattttaattaaagtgatgtcaaggaaggcctcaaggagaaggtgacagttgaatgaagagatgaaagaggtgaggggggaaatcatgtgaatatctggaggggagaatgctccaggcaggcagaggccctagtgtcttcagatgggagcatgaatggactgcacaagaaaccagaagggggcagccttgtcttatctcttttccaactggactgtccccttgttcactggaacaaattcaagctcctgtgcctggttctcaaagctgccaaaagaaggtggctgccaggaagaagatgccttccaagaagaaggtggctgcctctgtgctctggtcaagcagtttccctgatgtcagcaggctatcctctcctcaccagtgtatcatcactgcatctttcctcaaaggctttttattttctaatatccatcagcgcaaagtataaacaactttctttgcaaagctcaagactgactccttcaagaagccttccaagcttcatctcactacccatcagtgctctgcacataaagttcactccatttgagtccctaccatcttaacttgtacttgcccatgttatcttgatagtactgtattctctaggtattccatgtgggtatgtcttccctctccagctatatcaggaaatgaaccaatcatctgtatcatcccataatgcttaagaatgttatatattcaattggtgtttgacatgcatgggatggttggatggatgaatggatgtttggatggacagatgtaaggatggaaatggatggatgaatgaatggatagatagatgactggataaacagtact

SEQ ID NO: 3 - DNA, 12 bp, Homo sapiens
tggatggatgaa

SEQ ID NO: 4 - DNA, 1067 bp, Homo sapiens
gaacaaattcaagctcctgtgcctggttctcaaagctgccaaaagaaggtggctgccaggaagaagatgccttccaagaagaaggtggctgcctctgtgctctggtcaagcagtttccctgatgtcagcaggctatcctctcctcaccagtgtatcatcactgcatctttcctcaaaggctttttattttctaatatccatcagcgcaaagtataaacaactttctttgcaaagctcaagactgactccttcaagaagccttccaagcttcatctcactacccatcagtgctctgcacataaagttcactccatttgagtccctaccatcttaacttgtacttgcccatgttatcttgatagtactgtattctctaggtattccatgtgggtatgtcttccctctccagctatatcaggaaatgaaccaatcatctgtatcatcccataatgcttaagaatgttatatattcaattggtgtttgacatgcatgggatggttggatggatgaatggatgtttggatggacagatgtaaggatggaaatggatggatgaatgaatggatagatagatgactggataaacagtactaaaataagaagaattcttcagagtctgagtcagaagtttgactgctttctaaacacaatcaaaattaatttattctgaaaaaagtaaagtgtttaagaagctgaaaattgtaattcctccctcaaagacctgtctttctcctatttaccctcataatctggtgtaaatgcatctgtcagtggagacaggtttgagttccacagaagtctccaaaattagtccatgcattgacctcaaagaataagggtgtgttcagaaggatgcagggttttatgaccctaattctttgctgcttgtatcagccattttcagctgtatcagccatttccaactgtatcacatttctaaccatcaggacatgccatcatgccaagaggttgtttccaaagcagaataatgcccacgactaaaattggttttgctgtgccaactttctcactctttcaatttgtaaacacataagtgtttatttga

SEQ ID NO: 5 - DNA, 10 bp, Homo sapiens
cacacacatg

SEQ ID NO: 6 - DNA, 1056 bp, Homo sapiens
ttaatattgtcatttttatttcagtgggttctgggttaaatgcctaaatgaagaaagaggtttggcccttcataggaaaagatttgtggccaggtgcagtggctcacacctgtaattctagcactttgggaaaccgaggcgggtggatcacctgaggtcaggagttcgagaccagtctggccaacatggcaaaacccgtctctactaaaaatagaaaaattagcttggcatggtggcagacacctgtagtcctagctactcaggaggctgaggcaggagaattgcttgaacctgggaggtagaggttgcagtgagccgagatcactccattgcactccagcctgggcaacagagtgagactctgtctcaaaaaaaaaaagaaaagatttgtattcttcttttgtatctgcccccaaagaaagcagagtaacagctggggtgaattgccgtccatgaagaagaaatagacacagacatatgtgtggccatgtgcacgtgcacacacacatgcacacgcatgcacacatgtgcacacacacgcacacatgcacacacacatctatttgctcatgaaggaggttctgcagaatagtactttacaaaggaaagtcacatcatgttacttctctacttcaaaccttccagtggcttcccacatcacccagagtaaatgtcaatgttcttagaattgccctctaggccctatgccatttagccctgttctctctctgacctcctcatattgtgttggagtctgtgcgtactgtgtcatgagaaccatctgtgttcacttctttccaactctgccttcagggacggtaagttggtagcttgacatcggacattaagtatttacaccgtgcaaaatctacaggtgagagttttgttttttgtttgtttgttttttgagaaccactgtttccctcccccagctttcttttgtctacttcactccagccacagggggttccttgcagtgctgtaaacgtaagctttacaaaacagcttcagggcttctgaacctcacattcctctggctggaacactcttacccaa

SEQ ID NO: 7 - DNA, 11 bp, Homo sapiens
atttattaact

SEQ ID NO: 8 - DNA, 1025 bp, Homo sapiens
aaagtaaagtctgtgtctaattacatttttaaaaataaaaatgagaggagctctttataaaatgttttggcagtactaaaaagtacaaagaagaaagcaacgaattactccatgttgtaccatcaagttaaacaaaaaaaaagccgtaacatttggtgtatgacattccagacctcttatataagcatctacaagatagaaaaatgaatagacgaatggatggatggttgctatggatggataaatgaacaaatggatgactgagagagggaggaaggaaacaaaaaacaaggaaggaagaaaggaaggagggagaaaagaacaaagagaaaataaaaaatatgagaattgaatcatattatatctgttattttaaatgatggaaatatacttcaatttgatggaagaaacataaactgaaatgaactgaaggaagtgctaaacttcaaatagatgtttcacacaccctaagagatattagttcctgagggaactaattaatttattaactatttattaactattaattttggtatagattgattcattgctatgaaagatttaagcagctgttacctctcttatgtgtcttcctttctcaccttctctacattctgattttgttgattatataatttttaccatataaacatttttaataacatgtacataaattactatgattgtaattcttccaattgtttaaatctttatttagatatattttatgttcaacaccagtttcactaaagctgatccattcctgaattcttgatttatcacttggtgatattctgaactagaatgctgctatgacaaaatttccaattgtgtgtgcctttgtcttagtggacaggctgcatgtagccagcaaacaggtgccacaagactgacaggtagaaaaaatgataaagcattcgtaaaactgtcgccagctttacttgttcatcaagtacaatcatctggataatctggaaggccaaccaagtacttaccaaccttaaagctctaagagaaactgttag

SEQ ID NO: 9 - DNA, 19 bp, Homo sapiens
tgtatcagccatttccaac

SEQ ID NO: 10 - DNA, 1045 bp, Homo sapiens
tgggtatgtcttccctctccagctatatcaggaaatgaaccaatcatctgtatcatcccataatgcttaagaatgttatatattcaattggtgtttgacatgcatgggatggttggatggatgaatggatgtttggatggacagatgtaaggatggaaatggatggatgaatgaatggatagatagatgactggataaacagtactaaaataagaagaattcttcagagtctgagtcagaagtttgactgctttctaaacacaatcaaaattaatttattctgaaaaaagtaaagtgtttaagaagctgaaaattgtaattcctccctcaaagacctgtctttctcctatttaccctcataatctggtgtaaatgcatctgtcagtggagacaggtttgagttccacagaagtctccaaaattagtccatgcattgacctcaaagaataagggtgtgttcagaaggatgcagggttttatgaccctaattctttgctgcttgtatcagccattttcagctgtatcagccatttccaactgtatcacatttctaaccatcaggacatgccatcatgccaagaggttgtttccaaagcagaataatgcccacgactaaaattggttttgctgtgccaactttctcactctttcaatttgtaaacacataagtgtttatttgatcaaatagcacaggcatctaaagtatctcaataaatatcatccctttatttaggacatgtaatttttggatatccccaacatattgctcaacatgcacaccagcactagcaaaactccagagtgttctggccttgaataatgtgcttttcaaaaatagagacgactatctaccagtgtcagcttttccaggagaaatcttggctcaaataaaaggctgtttcaccacaatattagagattataagaaagctgaaaactggaagccgtagtttttgcccttctctgggacaagggctcctctgaaaatttgttgcaaaccatgaagtctctccagcagagtgcatacatatagataatataacacc

SEQ ID NO: 11 - DNA, 19 bp, Homo sapiens
ttttacaaagtaatatttg

SEQ ID NO: 12 - DNA, 1042 bp, Homo sapiens
ttgtttttgcctgttttcacattttttaatggactcatacaagatgtacacctttgtatctggcgtcttttgctcagcatgacatttctgaggtttgtccaagtttccgtttgttcctttttattgctaagtagtgtccattgtatggatgcatcacggtttgtttcacctgttgataactatttctagttttgatctgctgtgaacaaaactgctataaacattcttgtacaggtctttttgtgtacatatgtttttttcttctcttgggtcaatgcctagtagtggaatggctgagtcacacagtgggtgtatgtttaactttttaagtttaatagtgttccaaagttgttgcactgctttacattaccaacagcaatgcatgaaagttctagttgatcagaatactgacacttgctgacttttcttttatgctttatgctttaaatatagtttctatttgttttacacatttttacaaagtaatttgttttacaaatttttacaaagtaactttgttttacaaagtaatatttgttttataaaaaagttgcaaagatagtacagaaagttcccatatacctttcacctagttttctctaatgttagcatcttacattacatggtacattgccaagaccaggatatcgacactggtagatcactatgtactaaacttcagattttatttggatttcacccatttatccactaaccatcctttttctgttgcaggatcccatccaggataccacatttcatttagttgtcctgtcttcttagtctcctatggtgtgtgatagtttctcagtagtcccttatttttcataaccttcaaagtttgaagaatacgggtcagttatctggtagaatatcccttaatttggatctgtctgaagttacttttttcatttttagattaggactatgaattttggggaagactaccacaaagaccaagtgctcttgtcatcatgtcacctcagagggcacttgatatcaatatgacttatcacggatgatgttaaccttgattccttgc

SEQ ID NO: 13 - DNA, 27 bp, Homo sapiens
atttgttttacaaatttttacaaagta

SEQ ID NO: 14 - DNA, 1069 bp, Homo sapiens
tggccctgggcaaccactgttcagttttctgtcacttatagattgtttttgcctgttttcacattttttaatggactcatacaagatgtacacctttgtatctggcgtcttttgctcagcatgacatttctgaggtttgtccaagtttccgtttgttcctttttattgctaagtagtgtccattgtatggatgcatcacggtttgtttcacctgttgataactatttctagttttgatctgctgtgaacaaaactgctataaacattcttgtacaggtctttttgtgtacatatgtttttttcttctcttgggtcaatgcctagtagtggaatggctgagtcacacagtgggtgtatgtttaactttttaagtttaatagtgttccaaagttgttgcactgctttacattaccaacagcaatgcatgaaagttctagttgatcagaatactgacacttgctgacttttcttttatgctttatgctttaaatatagtttctatttgttttacacatttttacaaagtaatttgttttacaaatttttacaaagtaactttgttttacaaagtaatatttgttttataaaaaagttgcaaagatagtacagaaagttcccatatacctttcacctagttttctctaatgttagcatcttacattacatggtacattgccaagaccaggatatcgacactggtagatcactatgtactaaacttcagattttatttggatttcacccatttatccactaaccatcctttttctgttgcaggatcccatccaggataccacatttcatttagttgtcctgtcttcttagtctcctatggtgtgtgatagtttctcagtagtcccttatttttcataaccttcaaagtttgaagaatacgggtcagttatctggtagaatatcccttaatttggatctgtctgaagttacttttttcatttttagattaggactatgaattttggggaagactaccacaaagaccaagtgctcttgtcatcatgtcacctcagagggcacttgatatcaatatgacttatcacggatgatgtta

SEQ ID NO: 15 - DNA, 18 bp, Homo sapiens
ttcaaagcaaatgttgag

SEQ ID NO: 16 - DNA, 1036 bp, Homo sapiens
attctcccatctcactttctggtatgccctctccccaccttcctgcttgattttttccgcactaattatcactatctgacatactttatattataactatatgtttcattactgtctttctccttcactagaaggagaattgtatgtgggcagagaattgtatctgttttgttcacgactgtgttcttaactcctagaatcatgtcaggcacataggaggtgcttgatatttatcaaataaatattttctgtgtttttctaattacataaaagcaaaatacagagggttgataatacctttataagtttttataaatcctcccccaccccaaatattctggttctcttttcctttttttcagttagagatagctttttcactattggcaaaaagactgtctataaagtgtgtgacattgttgacttcggctttgattatctatgagactgtggcctcttcccacttgtttaagtgtcttggtaactctgagtggaaaaatttcaaagcaaagttgagttcaaagcaaatgttgagtgagagaatggaaaaaagtgcttttttccattctctcactcaacaacgatcaccacagaagacttctgtgaccaaatgtggagtggtttttccccacatattaagcaagcaagcaattctatagcagacatcagccatgtgtcctctgaattcaactcaattctgacagtatctacctggagataacttctgatcctacaggttgagggctcagtctccaagactgtcccccaccttcagatgtcagtcacaagtccagcttccagaatttctgaccaatctgcttccagttgatgttcctacaaccccttcaagtttgattgatttgctacagtggctcacagaactcagggaaacatgtttactgttttattataaaggatattacaaagggtacagaggaagaggtgcattgggcaagagttgtgagaaagggcatggagctttcatgccctccccaggcatgccattctcaaggaacctccagttgtgcagatatcc

SEQ ID NO: 17 - DNA, 15 bp, Homo sapiens
tgtttatgaaacaaa

SEQ ID NO: 18 - DNA, 1041 bp, Homo sapiens
taaactgaccagtcctgtgtagatgcctctgcaaatatattgggaaaaacatgaacttcaggacccaacttgcattaacctatccctgctagtcacttggggacaaagaaaggcctctcacccttagtatagggttgggaagcagccttacctccagagcttctagggaacccagaaccttggcatagagttcacaaagtttcttcaacctgtgaatgaaaagagacaaaagagaaaataaaggaaagaagggagggaaaaaaaaagtgggaaagaggcatttatcccctcataaaaaagattccatgagccattgattccactccagcatttcattttaattttttaaaacaaggcattttgttccatttcagatgtcttaaatctttgaggtcacaaatattaatcacacaggattgctggtaagtctttaagaaagtaaagtgacatgttgcctgaatcatggtattattttattattttaaatccaggagcccttttgtttatgaacaaaatgtttctgaaacaaatgtttatgaaaatgcctaagttcttctccctccctctcttctttctctctctttctgtctttttctttgtttttatacaaatgatgtaacttgccacaagcagaggaatggatagaaagagcaaagaaataagtacagaagtagaaccaaatatataaggaaatttagaattcataaaggtggacattttgaacccacagggaaaagacagttttcaataaaatggcactgggacaaatagctgctcgtgaaagaaataaagtaggatgcttacgttattttcttacaccaaaatatgtttcagaaggatcaaagatttacatataataggtcagaccataaaacgagtggaagaatgggtgatttctaaaaaattatcttggaatgagaaagtcttttctatgtatgatagatactcagaaaccatggttttaaaatggataaatttgattacataaaaagataaaattttattatagtataaaatgaataagccataaacaggatcaaa

SEQ ID NO: 19 - DNA, 21 bp, Homo sapiens
gatttcattcataagatacac

SEQ ID NO: 20 - DNA, 1041 bp, Homo sapiens
gattattgctcacttttcatcattttgtgctttcccatccacttgctaagaaagtgaggttcagaaaggagaagtgacctgtttggcatgacaaagtttgttattggtagaatcagagctgggacccaagtctcttcatgcccagtctagaactcattttgtttttataattttgtccccgtgttggcagttgtacagtggcatttgaggaagttttactgagtcctaactcagtataaggacacttcttctgacattgggtgaacccttggactacctaaaatgctactcttttttgatttttaatttttatgggtacagagtaggtgtatatatctatgggtacatgatgtgttttgatacaggcatgcaatgcgtaataatcatatcatgaagaatggggtaaaatgctattcttatgctgaaaccttctatggttatattgattggaattgctcttccagggaaaagcagcaccatgatgtatgtataattataatgatttcattcataagatacacgatttcattcataagatacaaaggaaattgggttgttttttttttttttttttgtatttataagaaacaagggagaagagctacctgaacctatttaggttttggattcaggtgcaggggatttctgtaacagcttagcatgcttttactctaccttccatattgagtcaggatcccaggggggaaataagttgtgtagcagcccccagagtactgctcctcacaccagttcttttcttcataatgcactggctgcaagatagagcaacaaaaacttgaaggagactcagagaaagctatttagcctagaataaatcgaacagtttagtaggctttgtattgtcaaaccaatgtatttcggaaatagagttgcattatcctgatgtttttaaagatatgtctttgggcaggtaaatgtccttggtaataagcttctaagaaattcagatcttagaatgtttttttcatttggtttctttgtatcttaagtactgttcataaattgcgataatctaattcc

SEQ ID NO: 21 - DNA, 17 bp, Homo sapiens
cttgctcagttacaaga

SEQ ID NO: 22 - DNA, 1034 bp, Homo sapiens
gaaaagcagagttcaatgcaaattctagaaatagttgtcaaaatccccatttcttatgtcctagataatacttgtatatttctggatgtccatagaaaaataaggatgtcattacatagaacaatagctgtcagcatacagaacaatagcagaacagtggggaggatttcagatgtgaacagtgcttgtgagaatgaagcaagctacagtgtcctccaaggggacttcgtgagctcaacttgacatttagtctcacatgactgccttaggctccttggcaccagtcaacacagaaggacattggatgtgtttatccaacacttctgtcttgccaacagagcagcatcagcagacagtcctcttcaggggaagagtcctcactgtatacagttgagatgtgaggaaatgacatccacgtggggtgggtccaccttgacctagaagccccttgctccatgggagccattatctctatgcttcataggcatagtgtccggtaccttgctcagttataagacttgctcagttacaagagacaccacactcagggaagcccaaagtgatagcctcctaccaggtatttacaattcatttagagtcctcccagcctggatgcagttcactaatgtggggatgggaggggaggcacttttagcataaatggtgttgcctccatagatggcattacaccttcatctggtttccagagaaacagagctagaaagttaatacaaggtcacatttgtccactgaaaaggaggaagtatttattaaccttttcgttacaggagtttttagtctgtttttttttcttcttaattgagtcagatcgcctttgttaacaatagcaggattgatgaactggattagttctggtatcaaagtagaagtctagaagacaagactaagatgaggaatagatgaaacccagggttctggaattggaaaaaggagtgccaacgggttgagatgaaggccatcagggtggggggcaggacatgcagcttgggagagttcatgaaactagctgtgga

SEQ ID NO: 23 - DNA, 1085 bp, Homo sapiens
aaatagaaaggttttcttggtcccaaaaggagctctgttgctatgaacattaatccctagtgtagaaattgtgttgatttaaaggagattcacttgttgagggaataagaaggatgctgggaagattctgattagaagaatattctgaagactagtcgagagatgacacagcaggagaaacctggagtgagattcagatgaaggaagaggcaagtttggccaaaaagctacttgtgaattttacatggaagacaccctggagatacttaaagggcattcaaactttaccaactggactgagataaaaagaatatcaaagaggcatctgtttatttgaaacaattattttcaggaggaaaaagttttcaaagaaattcacttaatgggtactttgaattgacacccatatgctttttgaaaagccatttcaaaggagaagtcatgagcacaaagaaagtgggtatgagaaataaggattgtgataggcaagtatcataattaccatcatcaccatcaccatcatcaccaccaccaccaccagtatcatcatgatcattaccaccatcaccatcatcatcatcatcatggttaacacttttttcacatttgctgcatactaaaatccttttaataagactttatacgtttatttttatttatctccctcaacaaccctataagcaaggtattattattttcattttttagattaagaaacagaggcacagagaggttaagaaacttgcctcaaatcacacagctagtaaatagcgagttcagaattcaaacccaggcccctctgggttctaagcctatgctctcagtatctaaaacagatggtctttttttttttttttttttttttttgagatggagtctcactctgtcacccaggctggagtgcagtggcatgatcttggctcactgcaacatccacctcccgggttccagtgattctcctgcctcagcctcccaagtaattgggattacaggtgtgtgccaccacacccggctaatttttgtatttttagtagaaacggggtttcaccatgttggccaggctgatctcaaactcctgacctcag

SEQ ID NO: 24 - DNA, 1052 bp, Homo sapiens
tataccacctggttcttcccacagggatacctcgacacagaaataaagggaacacattgttccaaggatttaaaataaataatatctgggagaattaacctgttgatggcacttaagagacacgcaaaaaaacccactttgggtcaatctgaaaacccgagtcagaagccaggtcgtgcattctgatagcatctttcaacggaatcagaataacttaaggttctcagtcaacagaaacttccccaaaactctgagtcattcaataaagatgcaccttctgttgatgttgtacaggcagagaaactagaattgtggaataagagatttcgtatttgttcttgtattaattctgtggccatgttggtcttctctctccaacaaggccataagattctcagggaagggttgagatacagctttatgtttcttctcagccaggattccttagagaaacagaaccaacaggagaaagagagagagagagagagagagatacatacatagatatatagatacatagatacatagatatagatagatgatattgatatagatatagatggagagagagacagagacagagagagaaacagagaaagagagaaggattcagatttattttaaggaattagctcatgttgattgagagggacggcaggtccaaaatctgtagggtaggtcacggacccaaggaagagttgcagtttgacttcaaaggcagtctgctggcagaattccctcttttcctgagagtcatcaatctttttctcttaaggacttcaactgattggataaagtccattcacattatggaagttaatctgctttgctcaaagtctactgatttaaatgttaaccttatcttaaaaatagcttgacagaaacattagaataatgtttgtccaaatatctacacaagttgacgtataaaattaactgtcacccttcttaacactattgaggtgtactttagctacaatgtactcattttattttagtttagtttccgtattttgttttttatagttagaagtaattttaaaaattttaac

(19) United States
(12) Patent Application Publication
(10) Pub. No.: US 2007/0259967 A1
(43) Pub. Date: Nov. 8, 2007

(54) Methods of Diagnosis

(76) Inventor: Anne T. Bruinvels, London (GB)

Correspondence Address: NIXON & VANDERHYE, PC, 901 NORTH GLEBE ROAD, 11TH FLOOR, ARLINGTON, VA, 22203, US

(21) Appl. No.: 11/661,211

(22) PCT Filed: Aug. 30, 2005
(86) PCT No.: PCT/GB05/03358

§ 371 Date: Jun. 29, 2007

(30) Foreign Application Priority Data
Aug. 27, 2004 (GB) 0419199.5

(51) Int. Cl. A61K 31/04; C07H 21/04; C12Q 1/68; G01N 33/53

(52) U.S. Cl. 514/741; 435/6; 435/7.4; 536/24.31; 536/24.33

(57) ABSTRACT

Methods for determining whether an ADHD patient is suitable for treatment with a monoamine oxidase type B (MAO-B) inhibitor, and uses of MAO-B inhibitors in medicaments for treatment of ADHD. Nucleic acid probes and primer sequences useful for determining MAO-B activity in ADHD patients.

### FIELD OF THE INVENTION

The present invention investigates the significance of levels of the monoamine oxidase type B enzyme in patients with Attention-Deficit Hyperactivity Disorders (ADHD) and presents a method for the determination of the suitability of a specific sub-group of ADHD patients for treatment with a monoamine oxidase type B inhibitor.

### BACKGROUND ART

Attention-Deficit Hyperactivity Disorder

Attention-deficit hyperactivity disorder (ADHD) is the most common childhood-onset behavioral disorder affecting approximately 5% of children and adolescents. Although it was previously believed that children eventually outgrew ADHD, it is now recognised that adults who, in hindsight fulfilled the conditions for diagnosis of the condition in childhood, can be diagnosed as having ADHD.

The condition is known to be highly heterogeneous and this has led to recent adjustments as detailed in the Diagnostic and Statistical Manual of Mental Disorders (DSM) IV. Following DSM IV, ADHD can be separated into three sub-types being: 1) inattentive, 2) hyperactive-impulsive and 3) combined inattentive and hyperactive-impulsive subtypes (Lahey et al., Am. J. Psychiatry, 1994, 151:1673), with the latter being twice as prevalent as the former two (25%:25%:50%) (Leung and Lemay, Adv. Ther., 2003, 20(6): 305; Biederman et al., Am. J. Psychiatry, 2002, 159:36; de Quiros et al. J. Dev. Behav. Pediatr., 1994, 5:311).

According to DSM IV, the following criteria are used in the diagnosis of the three ADHD subtypes:

Either (1) or (2):

(1) six (or more) of the following symptoms of inattention have persisted for at least 6 months to a degree that is maladaptive and inconsistent with developmental level:
(a) often fails to give close attention to details or makes careless mistakes in schoolwork, work, or other activities;
(b) often has difficulty sustaining attention in tasks or play activities;
(c) often does not seem to listen when spoken to directly;
(d) often does not follow through on instructions and fails to finish schoolwork, chores, or duties in the workplace (not due to oppositional behaviour or failure to understand instructions);
(e) often has difficulty organizing tasks and activities;
(f) often avoids, dislikes, or is reluctant to engage in tasks that require sustained mental effort (such as schoolwork or homework);
(g) often loses things necessary for tasks or activities (e.g., toys, school assignments, pencils, books, or tools);
(h) is often easily distracted by extraneous stimuli;
(i) is often forgetful in daily activities.

(2) six (or more) of the following symptoms of hyperactivity-impulsivity have persisted for at least 6 months to a degree that is maladaptive and inconsistent with developmental level:
Hyperactivity
(a) often fidgets with hands or feet or squirms in seat;
(b) often leaves seat in classroom or in other situations in which remaining seated is expected;
(c) often runs about or climbs excessively in situations in which it is inappropriate (in adolescents or adults, may be limited to subjective feelings of restlessness);
(d) often has difficulty playing or engaging in leisure activities quietly;
(e) is often “on the go” or often acts as if “driven by a motor”;
(f) often talks excessively.

Impulsivity
(g) often blurts out answers before questions have been completed;
(h) often has difficulty awaiting turn;
(i) often interrupts or intrudes on others (e.g., butts into conversations or games);

ADHD Predominantly Inattentive Type: if Criterion (1) is met but Criterion (2) is not met for the past 6 months.

ADHD Predominantly Hyperactive-Impulsive Type: if Criterion (2) is met but Criterion (1) is not met for the past 6 months.

ADHD Combined Type: if both Criteria (1) and (2) are met for the past 6 months.

Children with the predominantly inattentive subtype often experience considerable learning difficulties (American Academy of Pediatrics, Pediatrics, 2000, 105(5): 1158; August and Garfinkel, J. Am. Acad. Child Adolesc. Psychiatry, 1989, 5:739) resulting in serious academic underachievement. Furthermore, this subgroup of patients is often not treated with currently available treatments (Weiss et al., J. Attent. Disord., 2003, 7(1):1).

Concrete disease mechanisms have not yet been elucidated, although it is has been demonstrated that ADHD aggregates in families and it is expected that a significant genetic component underlies the disease (For review see Biederman, 1998, J. Clin. Psychiatry, 59(7):4). Genetic association studies using candidate genes have demonstrated a possible involvement of dopamine receptor- and transporter genes (Zametkin and Liotta, J. Clin. Psychiatry, 1998, 59(7):17; Hunt et al. Schiz. Bulletin, 1982, 8:236; Faraone and Biederman, J Atten Disord. 2002; 6(1):S7).

Shekim et al (Biolog. Psych. 18(6):707-714 (1983)) discuss the relevance of the metabolism of the catecholamines noradrenaline and/or dopamine in learning disabilities and suggest that affected patients may have a disturbance in the metabolism of these biogenic amines. In view of this, and other work, the first line treatment for ADHD is at present represented by psycho-stimulants such as methylphenidate and D-amphetamine. According to data available in 1998 (J. Clin. Psychiatry 59:7, 31-41 (1998)) between 2% and 2.5% of all school aged children in North America receive some pharmacological intervention for hyperactivity, with more than 90% being treated with the psychostimulant methylphenidate. Furthermore, according to Jodi Sarowitz in an article in The Chronicle Online 2002 (www.chronicle.duke.edu/vnews/display.v?TARGET=printable-&article id=3d817873efdfc), the number of prescriptions in the USA for methylphenidate is approximately 11 million per year, with an additional 6 million prescriptions for amphetamines.

Zametkin et al., (Amer. J. Psychiat. (1984) 141:9, 1055-1058) demonstrated that amphetamine, administered to children with ADHD produced a 1600% increase in the excretion of urinary phenylethylamine (PEA). PEA is a monoamine that can be isolated from the human brain and has a chemical structure almost identical to that of amphetamine.

However, drugs based on amphetamines and methamphetamines may cause a number of serious side-effects, including the possibility of addiction, sleeping problems and anorexia.

Other enzymes involved in the regulation of monoamine neurotransmission such as monoamine oxidase type A (MAO-A), dopamine beta-hydroxylase and catechol-O-methyl transferase may also play a role in the disease aetiology.

Molecular genetic and pharmacological studies suggest the involvement of dopaminergic and noradrenergic neurotransmitter systems in ADHD. Wender et al., (Psychiatry Res. (1983) 9:329-336) suggested that Attention Deficit Disorder (ADD)/ADHD may be caused by underactivity of the dopaminergic or phenylethyl-aminergic systems, with this underactivity being caused by a decreased rate of synthesis or an increased rate of breakdown of dopamine or PEA by the type B monoamine oxidase. Monoamine oxidase (MAO) A and B genes encode enzymes that participate in the metabolism of neurotransmitters of the dopaminergic and noradrenergic systems.

Investigations have been performed to determine the effects of administration of specific monoamine oxidase inhibitors (MAOI's), specifically selegiline and pargiline, as a means to reduce breakdown or increase synthesis of PEA and to determine the effects of this on the symptoms of ADHD.

In addition to this, it has recently been shown by Akhondzadeh et al., (Progress in Neuro-Psychopharmacol. & Biol. Psych. 27:841-845 (2003)) that selegiline (a selective MAO-B inhibitor) is metabolised to 1-amphetamine and 1-methamphetamine stimulant compounds and, as such, is valuable in the treatment of ADHD.

WO 97/17067 relates to the therapeutic application of selegiline against a number of diseases and conditions, including ADHD.

Shekim et al., (Am. J. Psychiatry, (1982) 139:936-938) suggested that low levels of MAO-B enzyme are a reflection of a generalised vulnerability to psychopathology and there are various studies that have investigated the relationship between MAO-A and MAO-B levels and various psychiatric disorders and personality traits, including ADHD.

Monoamine Oxidase Type-B (MAO-B) Enzyme

MAO-B is an enzyme responsible for the metabolism of the neurotransmitter dopamine as well as the biogenic trace amine, PEA. Dopamine is well known for its importance in a number of central nervous system (CNS) functions and it has been related to several diseases of the CNS, including Parkinson's disease, schizophrenia, addiction and depression. There has only been a limited amount of clinical research done on PEA, but it is thought to play a role in neurotransmitter release and is widely present in the central nervous system. MAO-B is primarily located on mitochondria and can be found in the brain as well as in peripheral organs and blood platelets. The MAO-A isozyme is the major type in fibroblasts and liver and mainly breaks down noradrenaline, adrenaline and serotonin, but is also capable of metabolising dopamine.

Control of MAO-B Activity

MAO-B activity is known to be under genetic control (Weinshilboum, in Neurogenetics: Genetic Approaches to the Nervous System, Ed. Breakefield, Elsevier, 1979, 257; Rice et al. Am. J. Hum. Genet., 1984, 36:36), is slightly increased with age and can be affected by drugs, including cigarette smoking. Generally, platelet MAO-B activity appears to be stable over time.

MAO-B, Dopamine and ADHD

MAO-B is widely expressed in the human brain in structures known to receive strong dopaminergic innervation such as frontal cortex, temporal cortex, caudate nucleus and putamen (Jossan et al. Brain Res., 1991, 547:69; Saura et al. J. Neurosci., 1992, 12:1977). It has been widely postulated that, in addition to a possible disturbance in noradrenergic neurotransmission, a deficiency in dopaminergic neurotransmission may underlie part of the pathology of ADHD.

A Neurochemical Basis for ADHD

It is of great interest that some reports have highlighted links between biochemical characteristics and specific patient phenotypes or disease subtypes. A study published by Shekim et al. (Shekim et al. Psychiatry Res., 1986, 18:179) highlights the correlation between reduced platelet MAO-B and reduced performance of children with ADHD in tests measuring hyperactivity and inattention. In this study, platelet MAO activity was examined in a sample of hyperactive and normal children. The results showed the hyperactive children to have significantly lower MAO activity than the normal children.

In contrast to the results of the Shekim study, Stoff et al., (J. Am. Acad. Child Psychiatry (1989) 28(5):754-760) suggested that MAO activity is elevated in boys with poorer performance on laboratory tasks requiring impulsivity. The authors suggested that the finding of a positive relationship between platelet MAO and impulsivity is consistent with several other reports of clinical improvement in hyperactive children treated with MAOI's (e.g. Zametkin et al., supra).

Jiang et al., (Am. J. Med. Gen. (Neuropsychiatric Genetics) (2001) 105:783-788) suggested that MAO genes may be susceptibility factors for ADHD and tested this hypothesis by investigating a linkage between ADHD and MAO genes. The results indicated that the MAO A gene may be a susceptibility factor for ADHD.

It is apparent from the above that effective and safe treatments for ADHD are required and that, although there has historically been some interest in the role played by MAO-A and B isoenzymes in i.a. ADHD, the precise relationship between those enzymes and ADHD is unclear. Furthermore, although a limited number of selective MAO-B inhibitors (selegiline and pargiline) have been used in the treatment of ADHD, the only rationale behind the use of these compounds is in their metabolism to the amphetamines/-methamphetamines currently providing the front line treatment for ADHD.

### SUMMARY OF THE INVENTION

The present invention provides for the first time evidence that low MAO-B activity in a patient is a risk factor for ADHD and more specifically for the predominantly inattentive sub-type of ADHD. Thus, the present invention is concerned with a method for determining whether a patient with ADHD is susceptible to treatment with a monoamine oxidase-B inhibitor, whereby the improvements in symptoms obtained via treatment with the MAO-B inhibitor are attributable to the action on the MAO-B enzyme rather than on the metabolism to amphetamines/methamphetamines.

Having determined that selective MAO-B inhibitors are appropriate for the treatment of such patients, the present invention also provides the use of MAO-B inhibitors hitherto unknown for the treatment of ADHD in such patients.

Accordingly the invention provides a method for determining whether an ADHD patient is suitable for treatment with a monoamine oxidase type B (MAO-B) inhibitor which comprises:
(a) determining ex vivo the level of MAO-B activity in an ADHD patient;
(b) if said activity of MAO-B falls within the 30% lower percentile of the full range of MAO-B activity within a normal population, testing said patient for symptoms of ADHD predominantly inattentive type (in accordance with DSM IV); and
(c) if said activity of MAO-B falls within the 30% lower percentile of the full range of MAO-B activity within a normal population and said patient tests for symptoms of ADHD predominantly inattentive type, concluding that said patient is suitable for treatment with an MAO-B inhibitor.

The invention additionally provides:
a method for determining whether an ADHD patient is suitable for treatment with an MAO-B inhibitor which comprises:
(a) selecting patients diagnosed with ADHD predominantly inattentive type in accordance with DSM IV;
(b) testing ex vivo the patients selected according to step (a) for activity of MAO-B; and

(c) submitting patients with an MAO-B level within the 30% lower percentile of the full range of MAO-B activity within a normal population for treatment with an MAO-B inhibitor;
use of an MAO-B inhibitor in the manufacture of a medicament for the treatment of a patient suitable for treatment therewith, wherein the suitability of the patient for such treatment is determined by:
(a) determining ex vivo the level of MAO-B activity in an ADHD patient;
(b) if said activity of MAO-B is within the 30% lower percentile of the full range of MAO-B activity within a normal population, testing said patient for symptoms of ADHD predominantly inattentive type (in accordance with DSM IV); and
(c) if said activity of MAO-B falls within the 30% lower percentile of the full range of MAO-B activity within a normal population and said patient tests for symptoms of ADHD predominantly inattentive type, concluding that said patient is suitable for treatment with a monoamine oxidase type B inhibitor;
use of an MAO-B inhibitor in the manufacture of a medicament for the treatment of a patient with ADHD, wherein the MAO-B inhibitor is other than selegiline, pargiline or rasagiline;
use of the flanking sequence of a VNTR sequence motif for the generation of a nucleic acid probe or primer for the assessment of a genotype predictive of MAO-B activity in an ADHD patient, wherein the VNTR motif is selected from GCTGCCAAGAAGAAGGTG, TGGATGGATGAA, ACCATCATC, CACACACATG, ATTTATTAACT, TGTATCAGCCATTTCCAAC, TTTTACAAAGTAATATTTG, ATTTGTTTTACAAATTTTTACAAAGTA, ATAGATAT, TTCAAAGCAAATGTTGAG, TGTTTATGAAACAAA, GATTTCATTCATAAGATACAC and CTTGCTCAGTTACAAGA, and wherein the flanking sequence is the sequence from 1 to 250 bases upstream or downstream of the motif;
a nucleic acid probe or primer for detection of a VNTR motif, comprising:
   (a) a fragment of the flanking sequence of the VNTR motif; or
   (b) a nucleic acid sequence complementary to (a);
      wherein the VNTR motif is selected from GCTGCCAAGAAGAAGGTG, TGGATGGATGAA, ACCATCATC, CACACACATG, ATTTATTAACT, TGTATCAGCCATTTCCAAC, TTTTACAAAGTAATATTTG, ATTTGTTTTACAAATTTTTACAAAGTA, ATAGATAT, TTCAAAGCAAATGTTGAG, TGTTTATGAAACAAA, GATTTCATTCATAAGATACAC and CTTGCTCAGTTACAAGA and wherein the flanking sequence is the sequence from 1 to 250 bases upstream or downstream of the motif;
a probe or primer of the invention for use in medicine;
use of a probe or primer of the invention for the in vitro determination of a genotype predictive of MAO-B activity in a sample from an ADHD patient;
a kit for use in diagnosing and/or treating ADHD in a subject which kit comprises a probe or primer of the invention.

### BRIEF DESCRIPTION OF THE FIGURES

FIG. 1: Table 3—a list of variable number tandem repeat (VNTR) motifs identified in the promoter region of the MAO-B and MAO-A genes and their surrounding sequences, useful in the design of primer and probe sets for use as (part of) a diagnostic test.

FIG. 2: Table 4—a list of primer sequences of single nucleotide polymorphisms (SNPs) identified in the MAO-B gene which may be used in the design of a diagnostic test.

### DETAILED DESCRIPTION

In its first aspect, the present invention provides a method for determining whether an ADHD patient is suitable for treatment with a monoamine oxidase type B (MAO-B) inhibitor which comprises:
a) determining the level of MAO-B activity in an ADHD patient;
b) if said activity of MAO-B falls within the 30% lower percentile of the full range of MAO-B activity within a normal population, testing said patient for symptoms of ADHD predominantly inattentive type (in accordance with DSM IV); and
c) if said activity of MAO-B falls within the 30% lower percentile of the full range of MAO-B activity within a normal population and said patient tests for symptoms of ADHD predominantly inattentive type, concluding that said patient is suitable for treatment with an MAO-B inhibitor.

The method of diagnosis of this aspect of, the invention can be supplemented by a method of treating the patient with an MAO-B inhibitor.

Alternatively, the present invention provides the use of an MAO-B inhibitor in the manufacture of a medicament for the treatment of a patient suitable for treatment therewith, wherein the suitability of the patient for such treatment is determined by:
a) determining the level of MAO-B activity in an ADHD patient;
b) if said activity of MAO-B is within the 30% lower percentile of the full range of MAO-B activity within a normal population, testing said patient for symptoms of ADHD predominantly inattentive type (in accordance with DSM IV); and
c) if said activity of MAO-B falls within the 30% lower percentile of the full range of MAO-B activity within a normal population and said patient tests for symptoms of ADHD predominantly inattentive type, concluding that said patient is suitable for treatment with a monoamine oxidase type B inhibitor.

In its second aspect, the present invention provides a method for determining whether an ADHD patient is suitable for treatment with an MAO-B inhibitor which comprises:
a) selecting patients diagnosed with ADHD predominantly inattentive type in accordance with DSM IV;
b) testing the patients selected according to step (a) for activity of MAO-B; and
c) submitting patients with an MAO-B level within the 30% lower percentile of the full range of MAO-B activity within a normal population for treatment with an MAO-B inhibitor.

The method of diagnosis of this aspect of the invention can be supplemented by a method of treating the patient with an MAO-B inhibitor.

In a third aspect, the present invention provides the use of an MAO-B inhibitor in the manufacture of a medicament for the treatment of a patient with ADHD, wherein the MAO-B inhibitor is other than selegiline, pargiline or rasagiline.

By screening human public domain data for novel associations between biological effects (e.g. protein activity) and a number of central nervous system disorders it was unexpectedly revealed that low MAO-B activity may represent a risk factor for ADHD. Table 1 lists the data obtained. Although approximately 1 in 10 people without ADHD have low MAO-B activity, the prevalence of that effect is far greater in ADHD patients.

TABLE 1
Platelet MAO-B Activity in Children with ADHD and
Control Subjects*

| | ALL | Low** | High |
|---|---|---|---|
| ALL | | 26 | 18 |
| Control | 12 | 8% | 92% |
| ADHD | 19 | 84% | 16% |
| Relative Risk: | 10.11 | Control: | 8% |
| | | ADHD | 84% |

*Data (used and transformed) from Shekim et al. Am. J. Psychiatry, 1982, 139: 937; Shekim et al. Psychiatry Research, 1986, 18: 179; Garpenstrand et al. J. Neural Transm, 2001, 107: 523
**Low <30 nmol/ml/h/number of platelets × 10⁻⁶

Therefore, low MAO-B activity, i.e. an activity within the 30% lower percentile of the full range of MAO-B activity within a normal population, may represent a possible biological risk factor for the disease as it is more frequently expressed in the patient population than in the control group. (Similarly, the APOE-ε4 allele, a known risk factor for Alzheimer's disease, appears in 13% of the general population and in 35% of patients (Farrer et al. JAMA, 1997, 278:1349).

Therefore, it is suggested that individuals with low MAO-B activity may have a higher chance of developing ADHD. However, as with many psychiatric conditions, ADHD is most likely to be caused by a number of risk factors of which low MAO-B activity may be one. Other risk factors for ADHD have been suggested, although none have been confirmed. These include dopamine receptor genes, the dopamine transporter as well as the MAO-A enzyme.

Based on the biochemical studies by Shekim et al. (Shekim et al. Psychiatry Res., 1986, 18:179 and Shekim et al. Am. J. Psychiatry, 1982, 139:485) the present inventors reasoned that different biochemical characteristics may be associated with different ADHD subtypes.

Thus, for example, the hyperactive and combined subtypes may be characterised by:
Low levels of the noradrenaline metabolite 3-methoxy-4-hydroxyphenylglycol (MHPG)
Reduced noradrenergic neurotransmission (putatively mediated via MAO-A)
No significant change in levels of the dopamine metabolite homovanillic acid (HVA)
Normal dopaminergic neurotransmission.

The inattentive subtype, may be characterised by:
Low MAO-B activity and resulting low HVA levels
Reduced dopaminergic neurotransmission
No significant change in MHPG levels
Normal noradrenergic neurotransmission
Possibly reduced turnover of phenylethylamine (and its active metabolite phenylethanolamine).

The present inventors consider that there is a correlation between ADHD patients with the predominantly inattentive subtype and low MAO-B activity. The inattentive subtype is generally seen as the more severe form of the disease, with a greater deficit in the social and cognitive functioning of the patient. The inventors consider that selective modulation of MAO-B activity may represent an effective and safe treatment for specifically those ADHD patients with low MAO-B activity.

As used herein, a patient having ‘predominantly inattentive symptoms’ or patient with ‘ADHD predominantly inattentive type’ is a patient who has been so diagnosed according to the criteria set forth in DSM IV, and as set out above. This classification does not include patients with predominantly hyperactive-impulsive or combined symptoms.

A ‘patient’, as used herein is intended to embrace patients of any age, be they child, adolescent or adult patients. Whilst diagnosis of adults may be via hindsight recognition of symptoms suffered during childhood, such adults may still be diagnosed as suffering from ADHD.

In the method of the first aspect of the invention, a patient diagnosed with ADHD is tested to determine MAO-B activity. The diagnosis of ADHD may have been made at any time during the patient's history and need not be of any particular sub-type of ADHD. Any such ADHD-diagnosed patient with a low MAO-B activity level, i.e. an activity level within the 30% lower percentile of the full range of MAO-B activity within a normal population, is then tested to determine whether the ADHD is of the predominantly inattentive type according to the symptoms set out in DSM IV. If this latter test is positive, the patient can then be treated with an MAO-B inhibitor.

In the method of the second aspect of the invention, a patient diagnosed with ADHD is selected according to a determination of predominantly inattentive type and said patient is then tested for activity of MAO-B. If the MAO-B activity level is within the 30% lower percentile of the full range of MAO-B activity within a normal population, said patient is then submitted for treatment with an MAO-B inhibitor.

In accordance with the invention, the level of MAO-B activity in a subject may be determined for example, ex vivo. For example, activity may be determined using an in vitro test, such as one carried out on a sample which has been taken from the subject.

The exact magnitude of MAO-B enzyme activity is dependent on the experimental conditions, data read-out and analysis used in the determination of enzyme activity. However, the activity in the patient relative to the activity in the normal population should not be dependent on the method of determination of enzyme activity.

MAO-B activity can be directly measured in various ways as illustrated in the Examples hereinafter, in blood, platelets, brain biopsies, cerebrospinal fluid (CSF), lymphocytes, liver or other tissue samples.

For the purposes of the present invention, measurement of MAO-B activity is preferably in platelets.

Classically, MAO-B activity is measured in platelets as described, for example, by Wurtman and Axelrod (Biochem Pharmacol. (1963) 12:1417-1419), Jackman et al. (Clin Chim Acta. (1979) 96(1-2):15-23), Shekim et al. (1982, 1986, supra, Shekim et al., Psychiatry Res. (1984) 11(2):99-106), Young et al. (Arch Gen Psychiatry (1986) 43(6):604-609), Hallman et al. (Acta Psychiatr. Scand. (1987) 76(3):225-34), Garpenstrand et al. (J Neural Transm. (2000) 107(5):523-530), Whitfield et al. (Psychol Med. (2000) 30(2):443-54).

In accordance with this aspect of the present invention, MAO-B activity may be measured by:

(A) direct biochemical tests, such as:
1) radiometry, in platelets;
2) radiometry, in tissue samples;
3) gas chromatography-mass spectrometry (GC-MS);
4) spectrophotometry;
5) luminometry
6) high throughput fluorescence assay;
7) high performance liquid chromatography (HPLC);
8) Berthelot Reaction;
9) Immunoblotting;
10) Radiolabelling;
11) Positron emission tomography; or

(B) indirect biochemical tests, such as:
1) Urine Concentrations By GC-MS;
2) HVA Concentrations in Urine Samples;
3) Urine and Plasma Concentrations of PEA and related substances;
4) Plasma PEA Concentrations by GC-MS;
5) Urine Concentrations of PEA & Related Substances by GC-MS;
6) Urine concentrations of HVA by GC; or
7) HVA In Cerebro-Spinal Fluid (CSF) by GC-MS; or

(C) by genetic tests in any tissue sample of fluid, including blood and saliva, for example including measuring to select those ADHD patients with genotypes associated with low MAO-B activity, such as the G-allele, as defined hereinafter.

Various ways to express MAO-B enzyme activity may be used. For example, the 30% lower percentile of the full range of MAO-B activity within a normal population may be defined as ≦30 nmol/ml/h/number of platelets×10⁻⁶ (following measurements by Shekim et al., 1982, 1984, 1986, supra) or ≦8 nmol/min/10¹⁰ platelets (as by Garpenstrand et al., supra), or ≦3 MAO-B units/10⁸ platelets (as by Young et al., supra).

In the present invention, it is preferred that MAO-B levels are defined as less than or equal to 30 nmol/ml/h/number of platelets×10⁻⁶ (following measurements by Shekim et al., 1982, 1984, 1986, supra); or less than or equal to 8 nmol/min/10¹⁰ platelets (as by Garpenstrand et al., supra), or less than or equal to 3 MAO-B units/10⁸ platelets (as by Young et al., supra)

Although any of the above methods may be used to measure MAO-B activity, or any other method available from the art, it is preferred to measure MAO-B activity by radiometry, for example, radiometry in platelets or by gas chromatography/mass spectrometry or by genetic tests.

The most preferred method for assessment of MAO-B activity in the methods of the present invention is by a genetic test.

Preferred genetic tests include, for example:
by use of one or more probes/primers for variable number tandem repeats (VNTRs) and/or single nucleotide polymorphisms (SNPs);
by the presence of a G-allele in the MAO-B intron 13;
by the assessment of the genotype at the AP-2beta gene;
by the assessment of the genotype at or expression levels of the c-Jun, Egr-1 and Sp1 genes;
by the assessment of genes and proteins on the protein kinase C and MAPK signalling pathways; and/or
by the assessment of genes or proteins involved in dopamine and PEA turnover.

The most preferred such test is the determination of the presence of the G-allele in the MAO-B intron 13.

MAO-B enzyme activity is under genetic control, and therefore a combination of genotypes and/or gene expression levels may be assessed to identify those patients with low MAO-B activity.

MAO-B activity was demonstrated to be largely hereditary (Rice et al., Am. J. Hum. Genet. (1984) 36(1):36-43, Pederson et al. 1993, Psychiatry Res., 46(3):239) and is thought to be genetically controlled (Weinshilboum, Neurogenetics: Genetic Approaches to the Nervous System, Ed. Breakefield, Elsevier, 1979, 257; Rice et al. supra). Genetic tests can be used to select ADHD patients with genotype and/or gene expression patterns resulting in low MAO-B activity. The MAO-B gene is located on chromosome Xp11.3 and has 15 exons, encoding a 519aa product. It is found adjacent to the MAO-A gene and expression of the MAO-B gene may be coordinately regulated with the MAO-A gene. Other genes may also influence MAO-B gene expression and/or activity.

The MAO-B gene has multiple Variable Number Tandem Repeats (VNTRs) and Single Nucleotide Polymorphisms (SNPs), which used alone or in combination, may be useful to identify the ADHD subgroups with low MAO-B activity and/or the predominant inattentive subtype of the disease. Specifically, variances in the MAO-B intron 13 were demonstrated to correlate with activity of the enzyme (Garpenstrand et al., J. Neural Transm. (2000) 107(5):523-530; Balciuniene et al. 2002, Hum Genet., 110(1):1). The G-allele was found to associate with low MAO-B activity.

Therefore, the present test would include such a measurement to select those ADHD patients with G-alleles for treatment with a MAO-B inhibitor.

Further possibilities of genotypes which may associate with low activity of the MAO-B enzyme include not only variances in the MAO-B but possibly also in the MAO-A enzyme, which have neighbouring chromosomal localizations and the expression of which may be linked by common pathways.

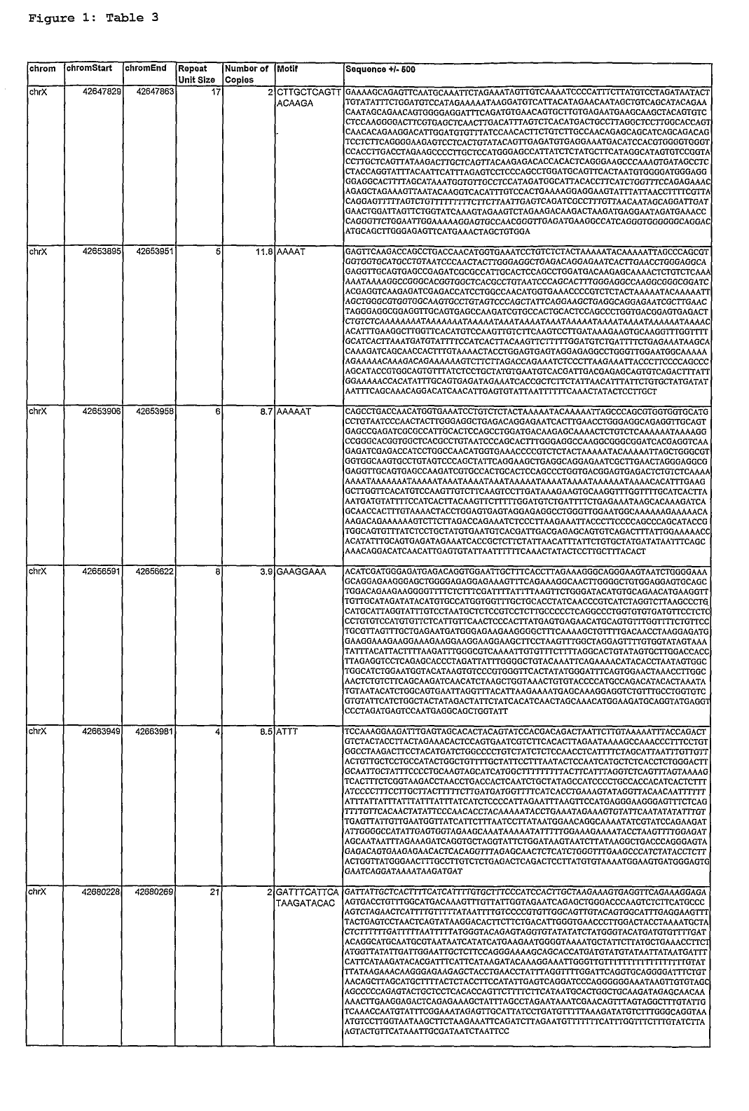

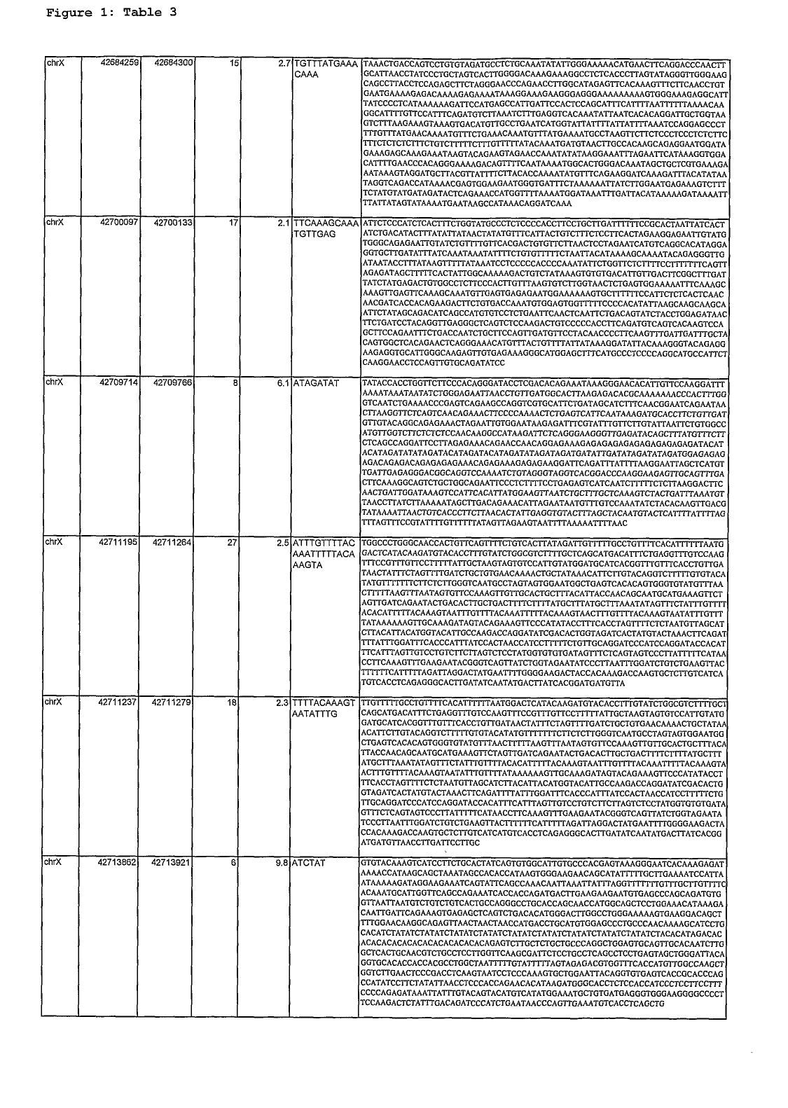

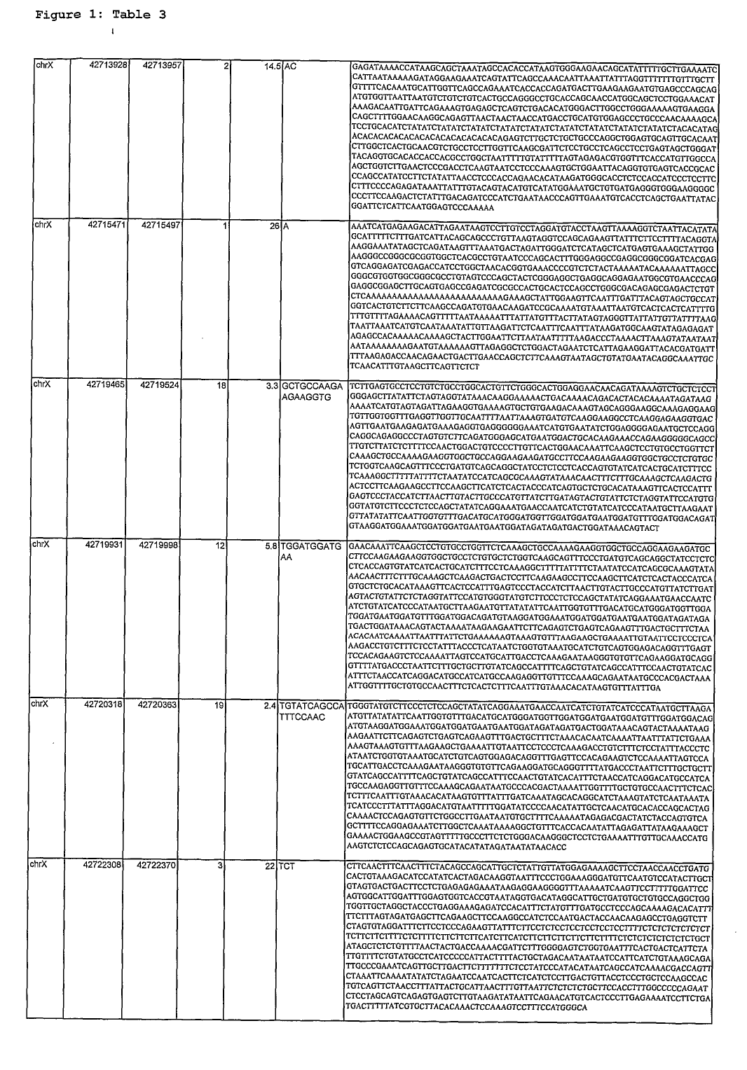

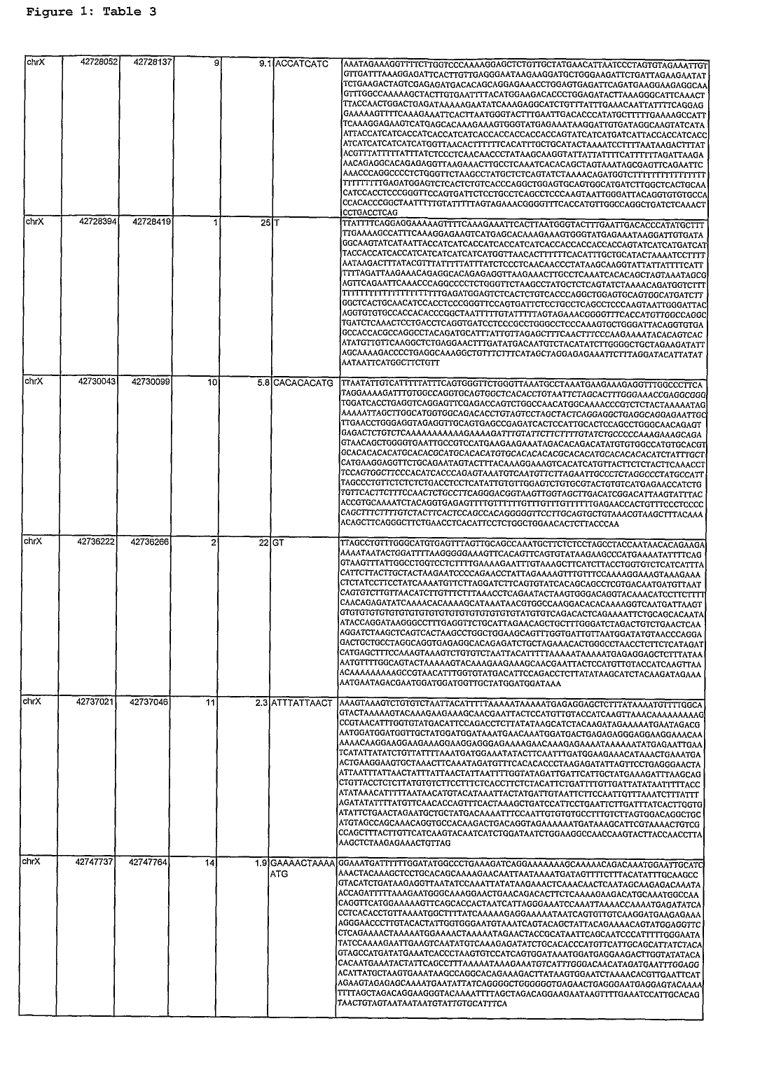

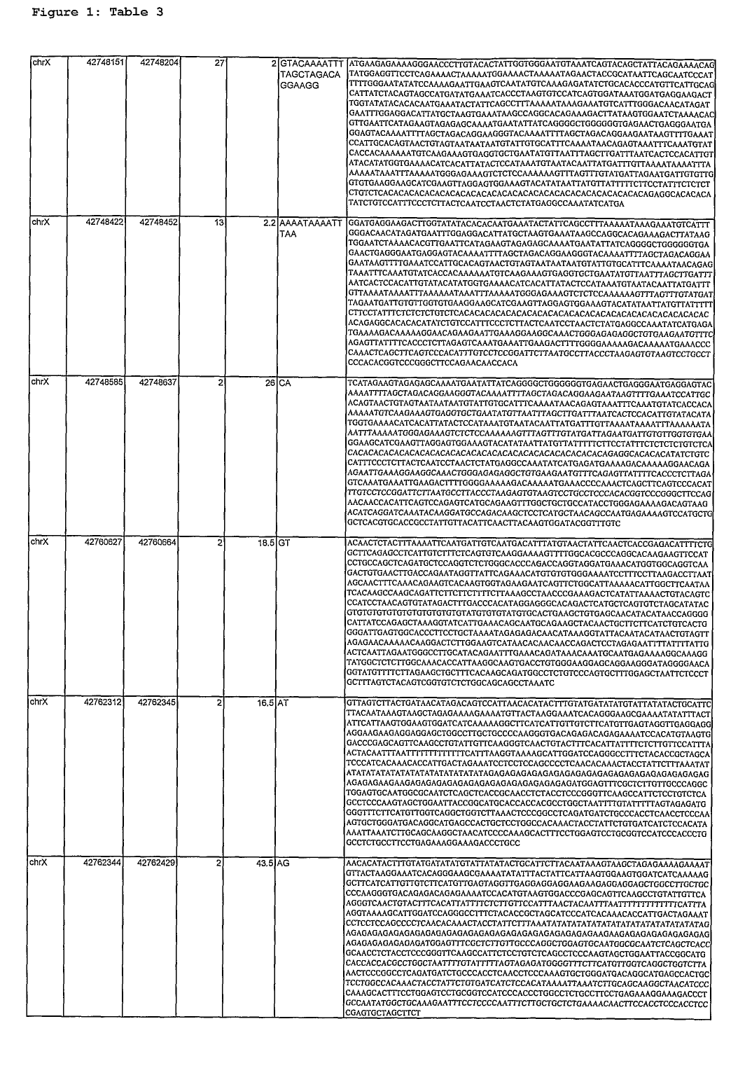

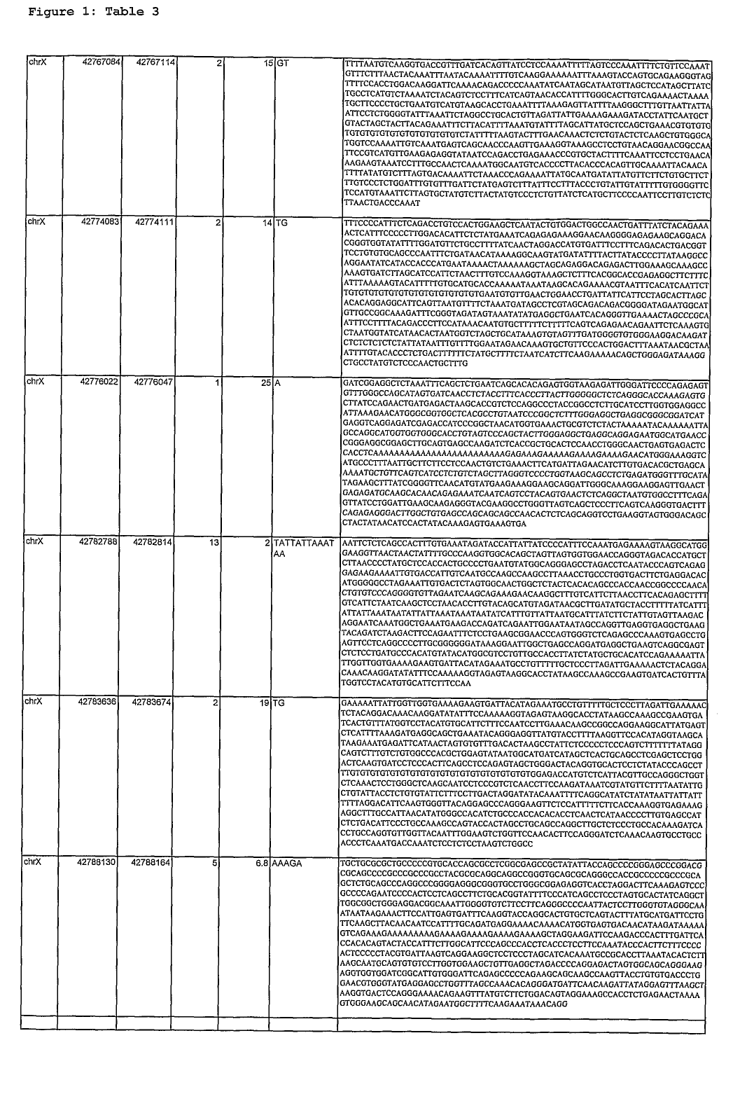

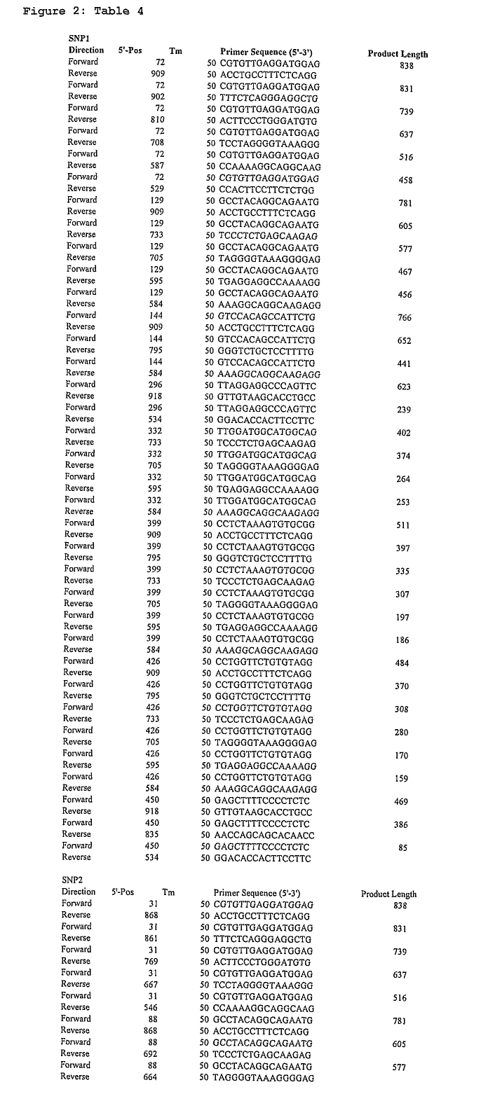

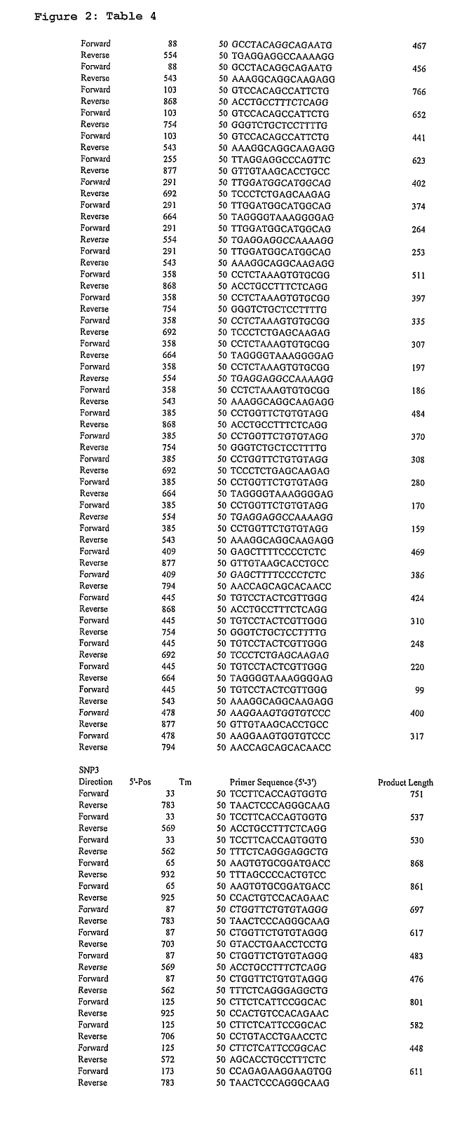

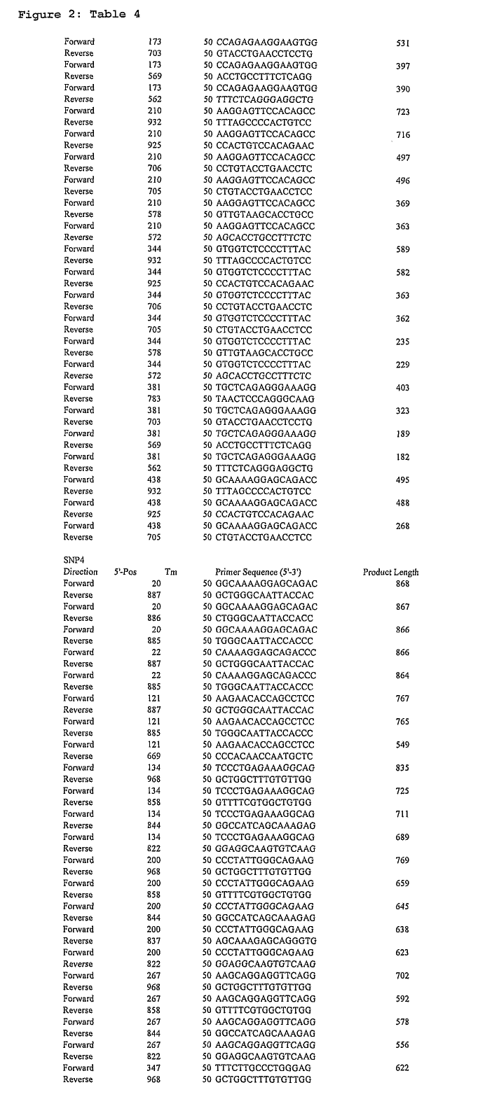

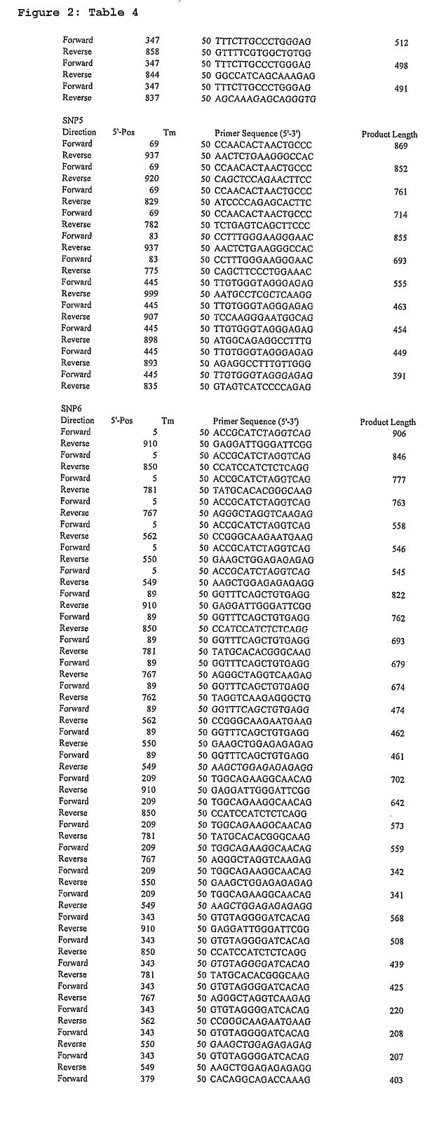

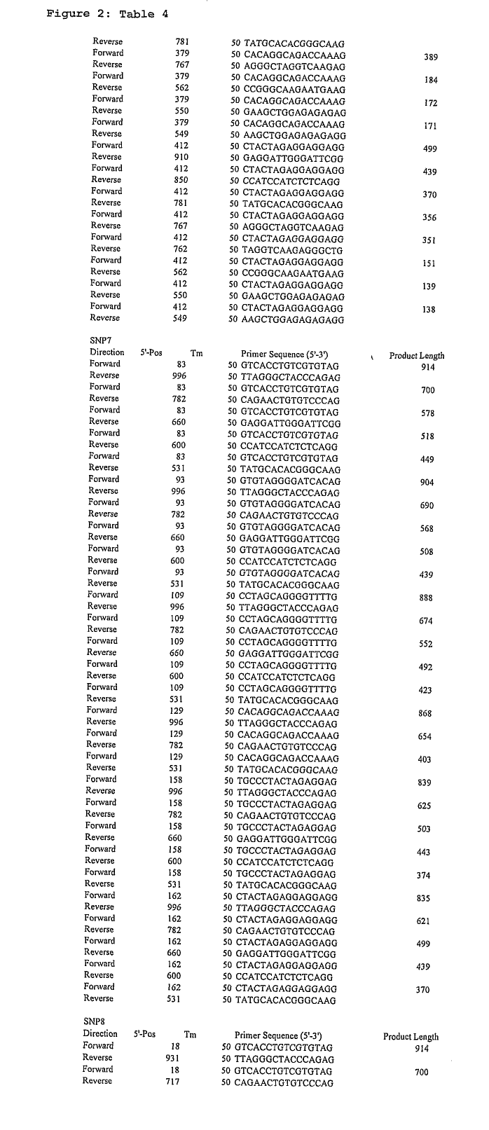

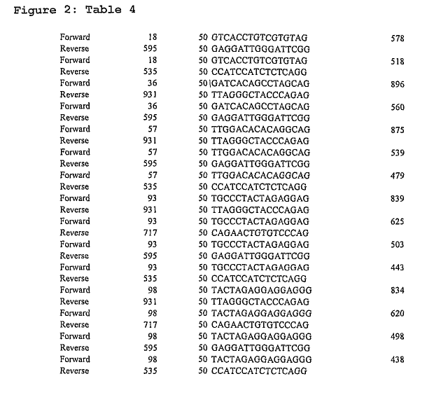

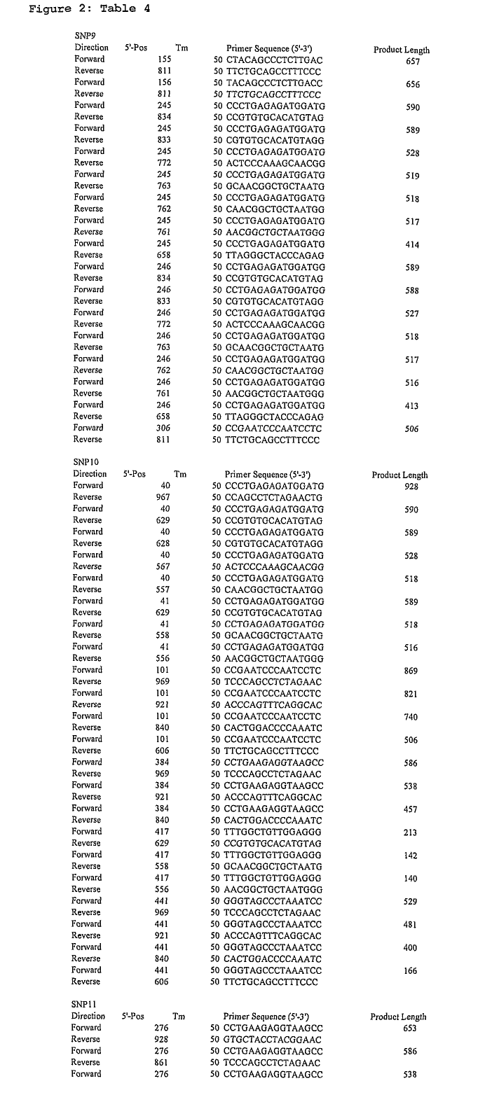

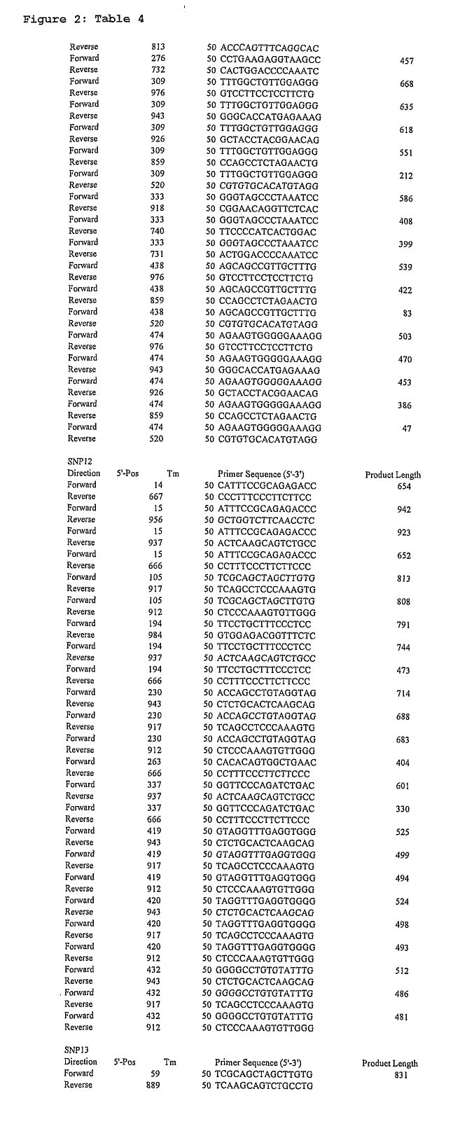

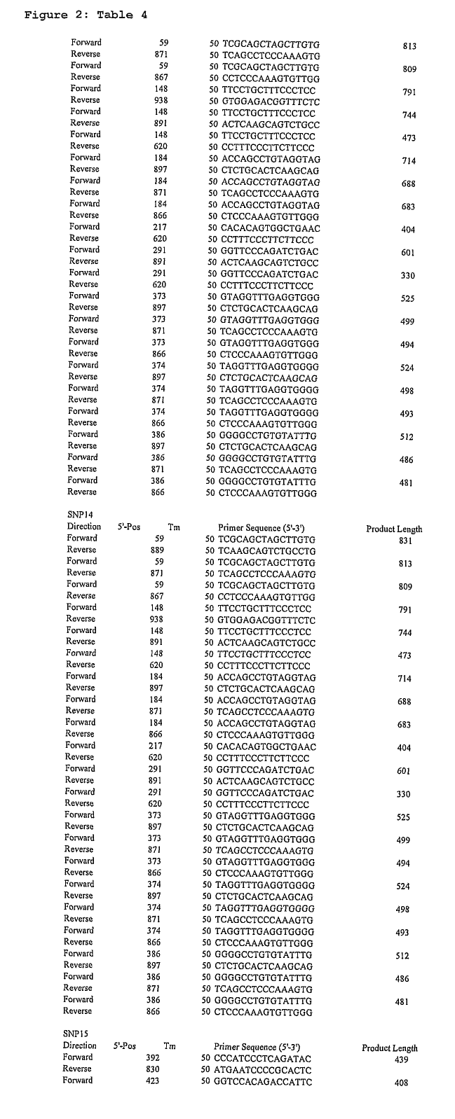

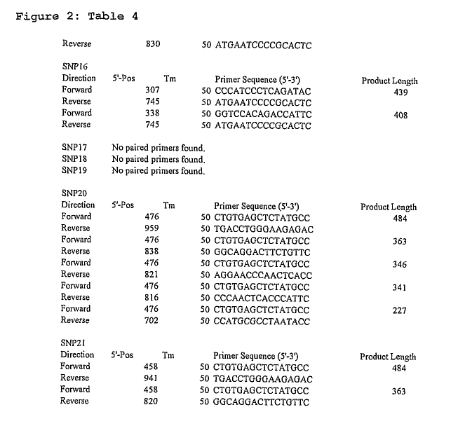

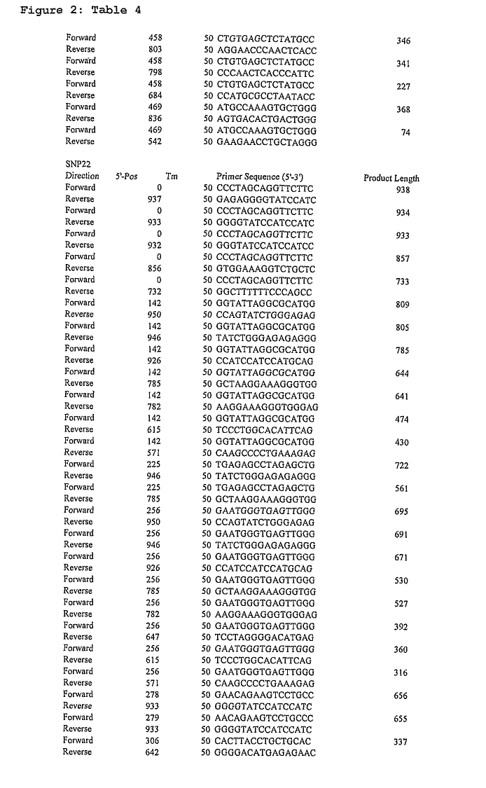

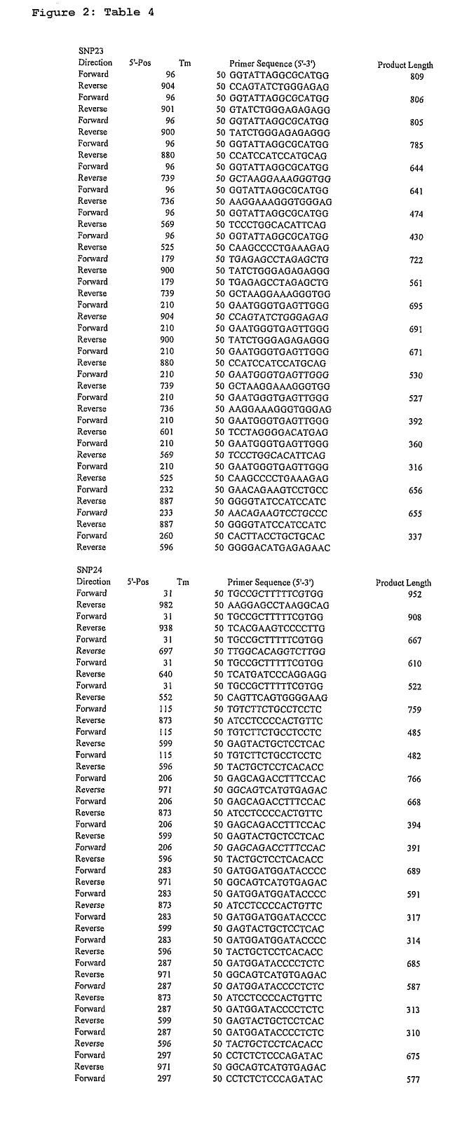

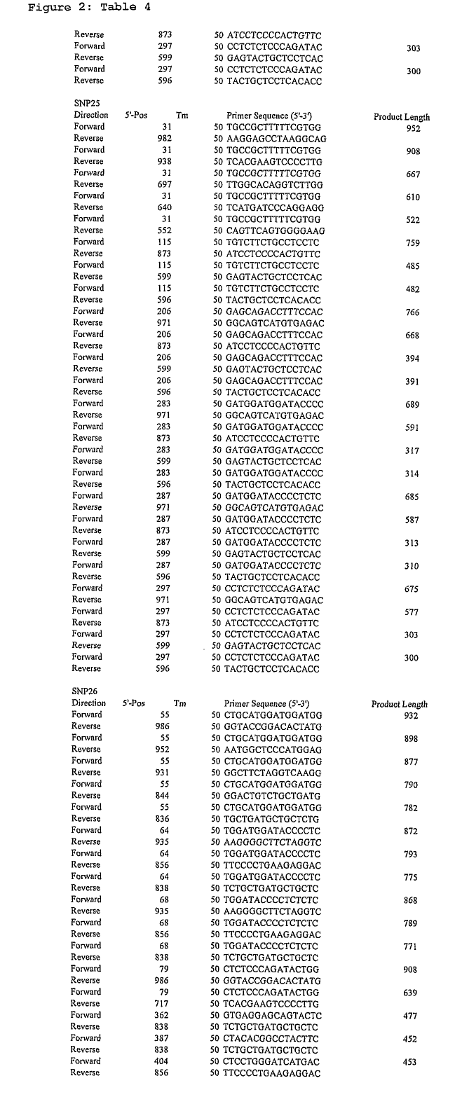

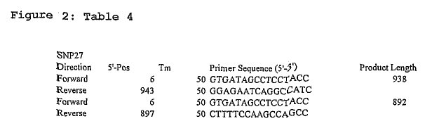

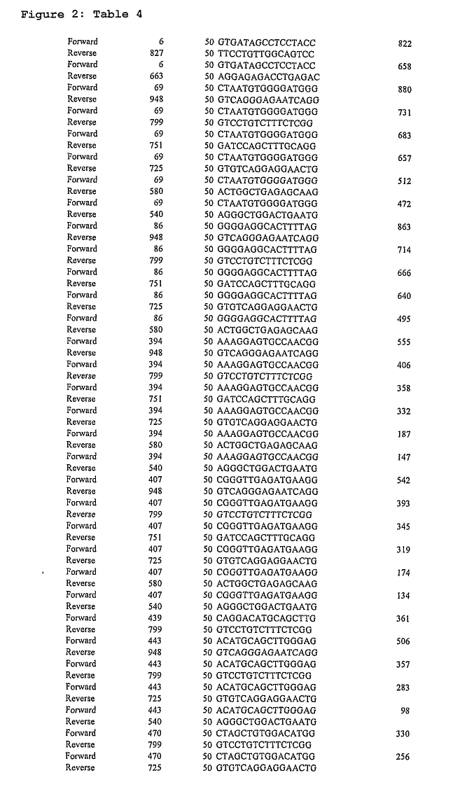

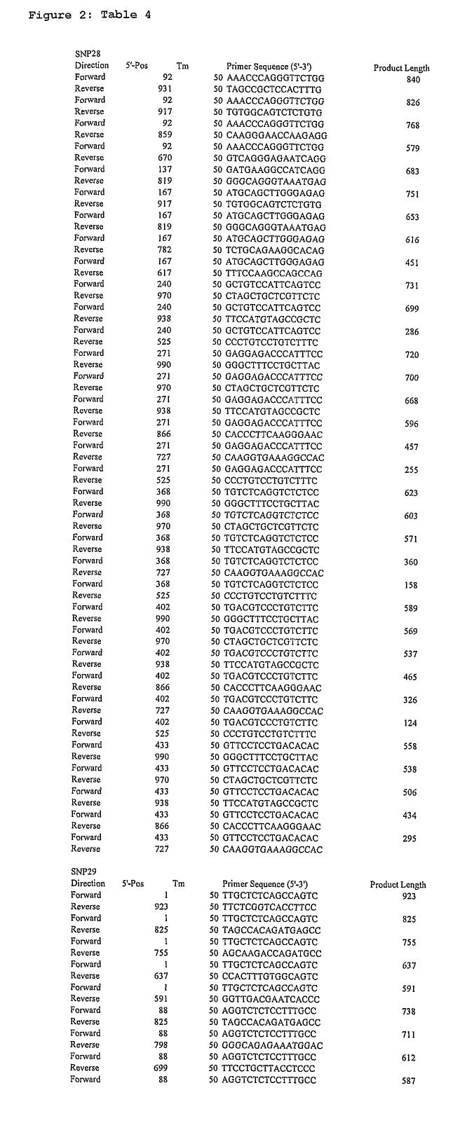

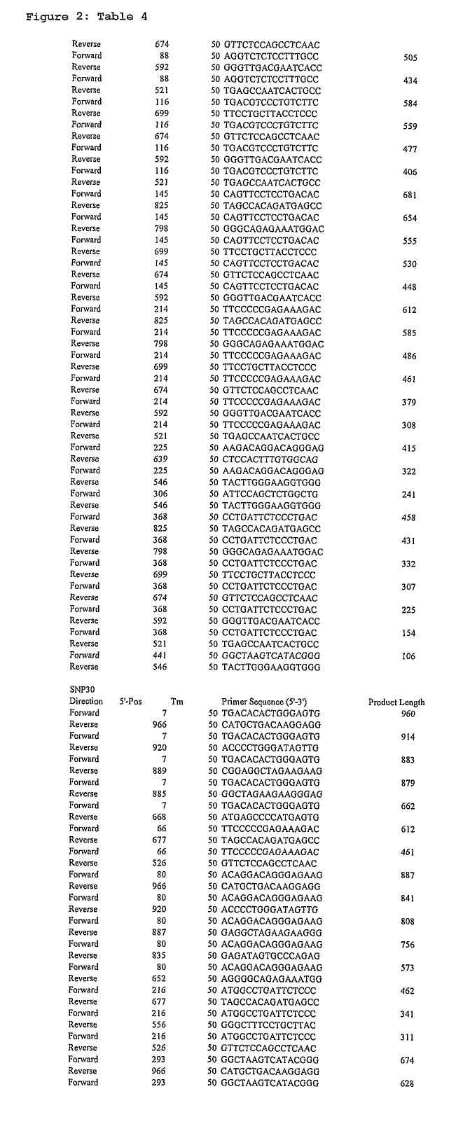

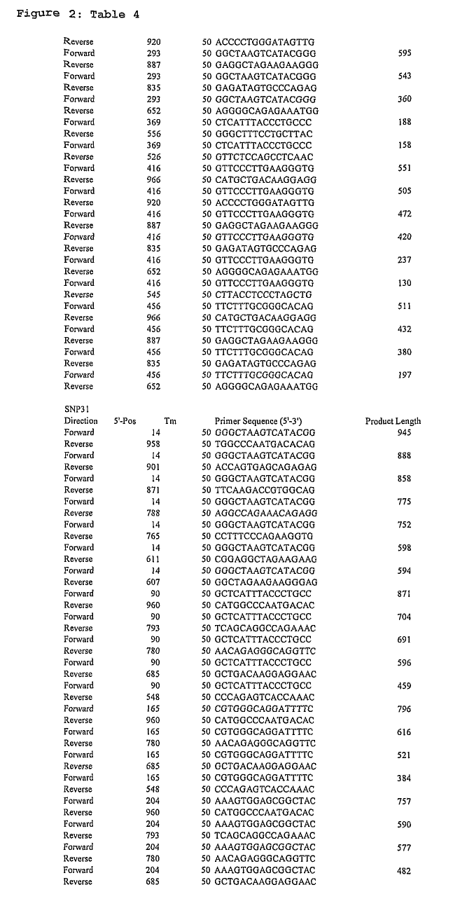

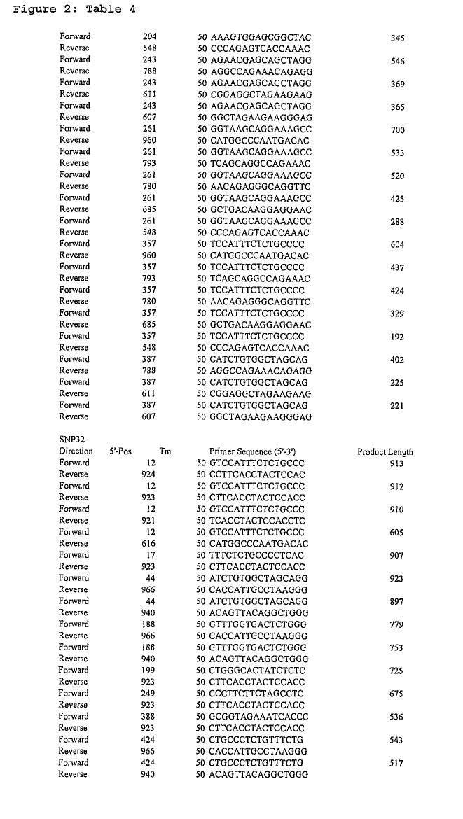

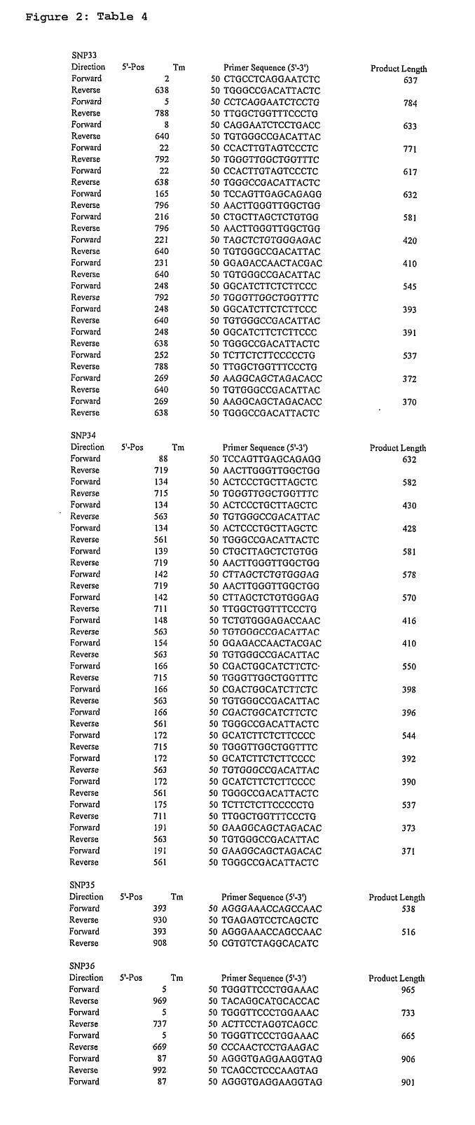

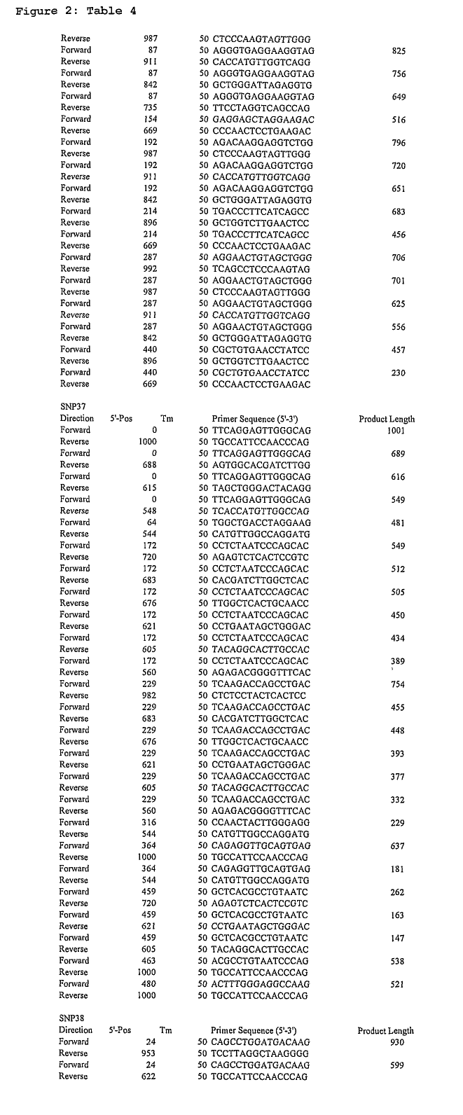

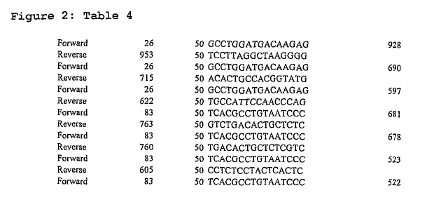

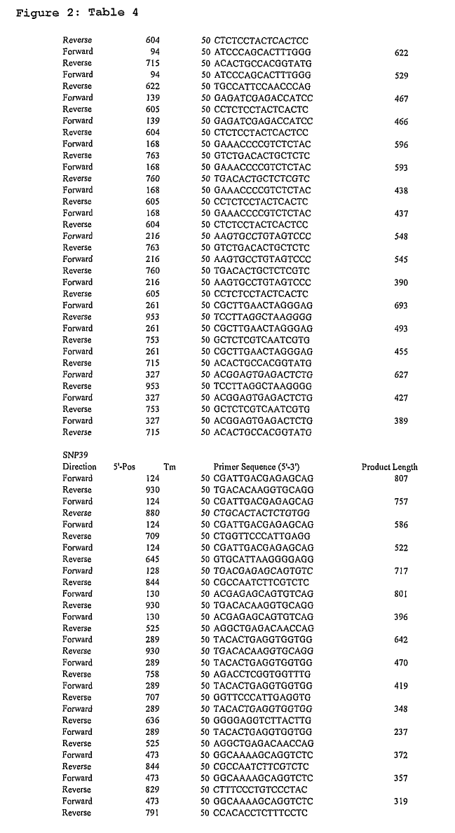

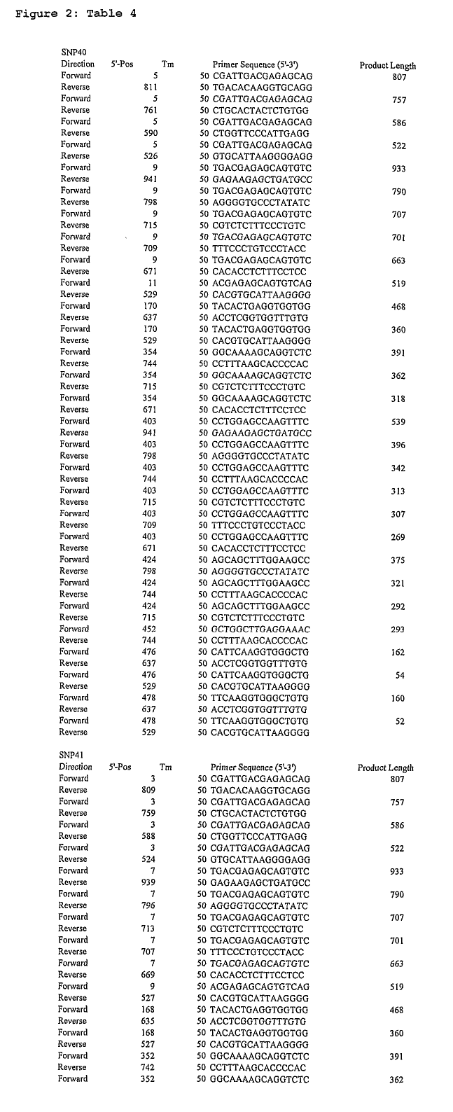

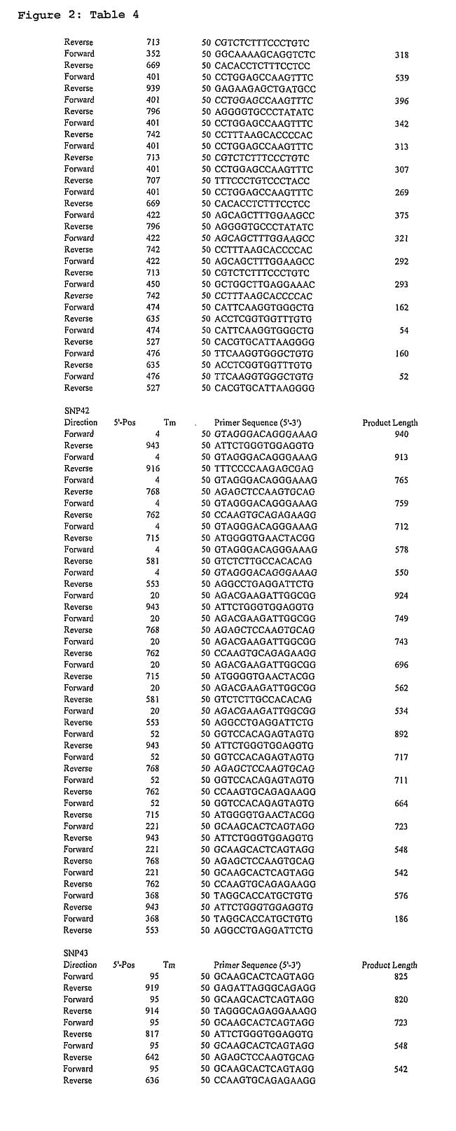

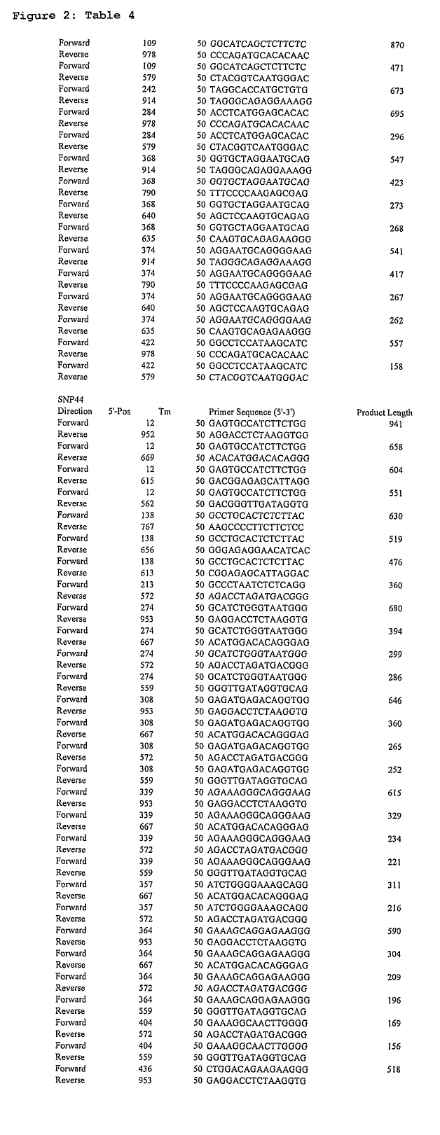

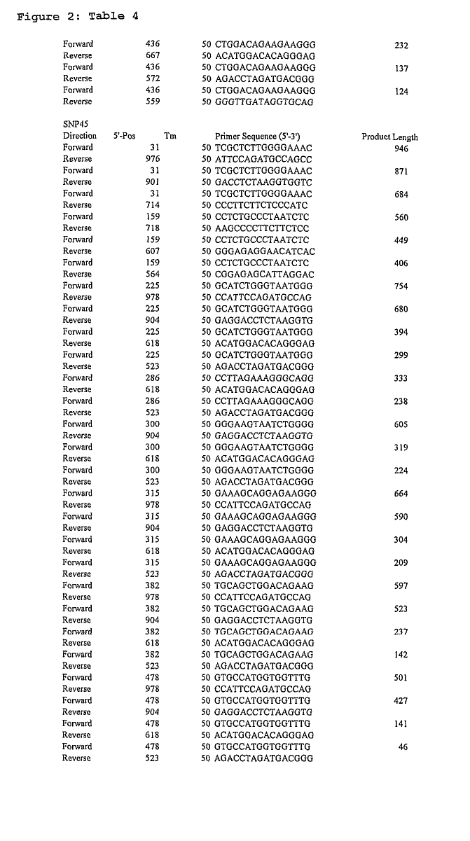

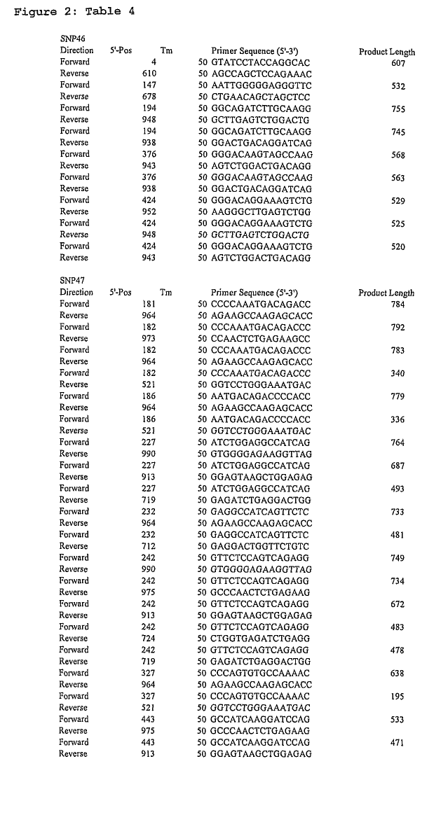

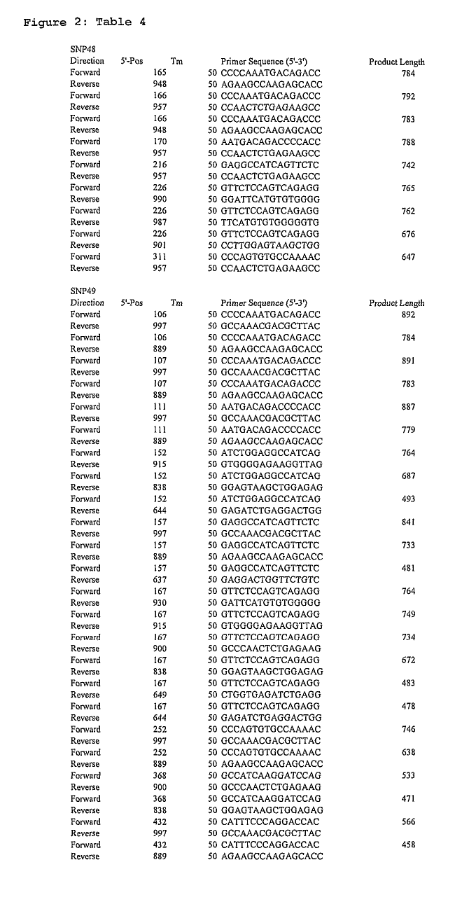

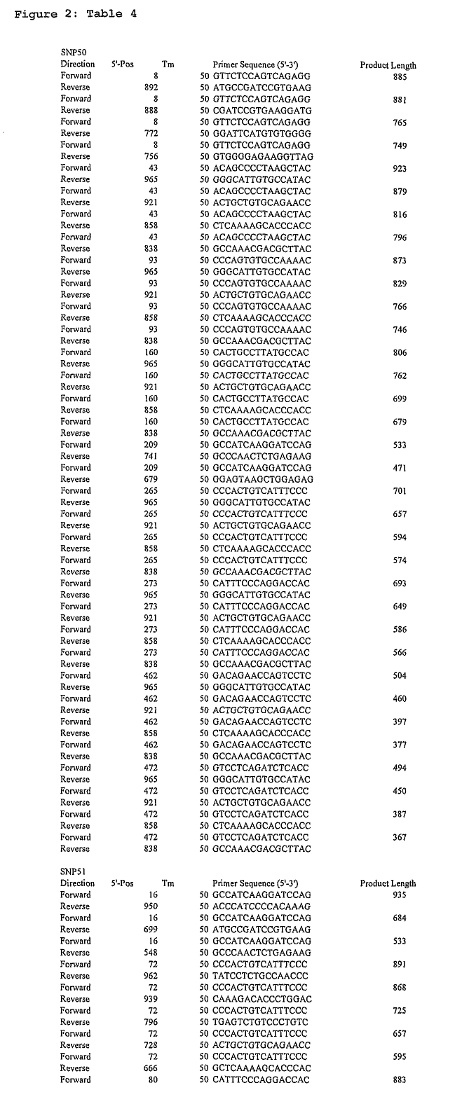

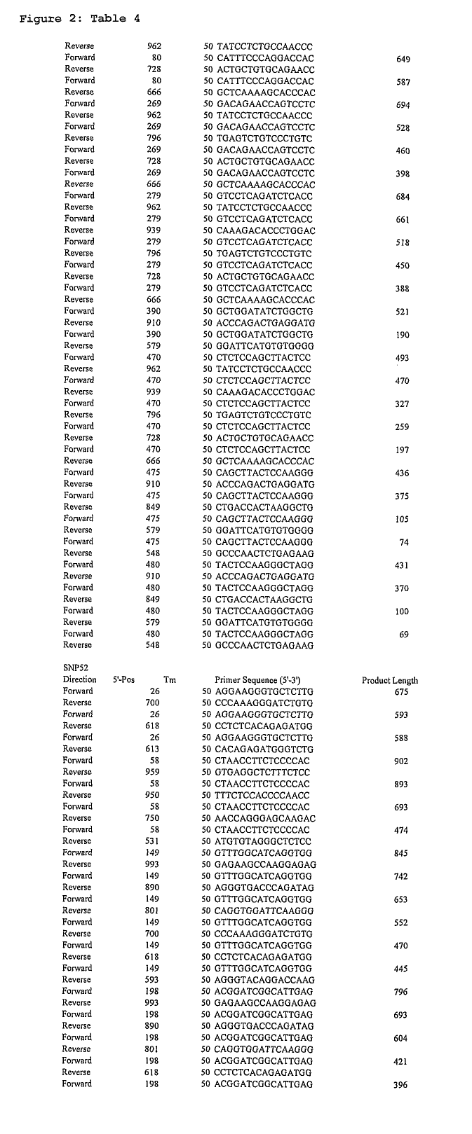

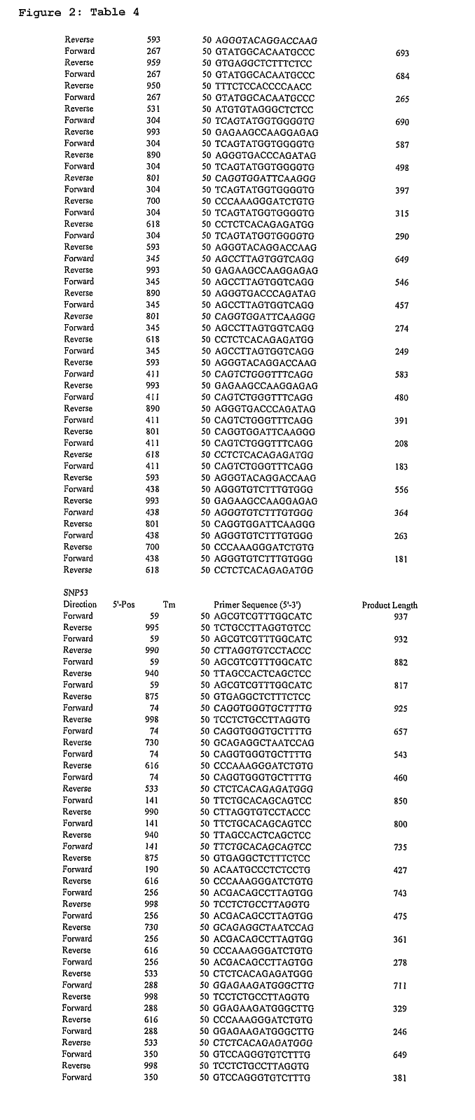

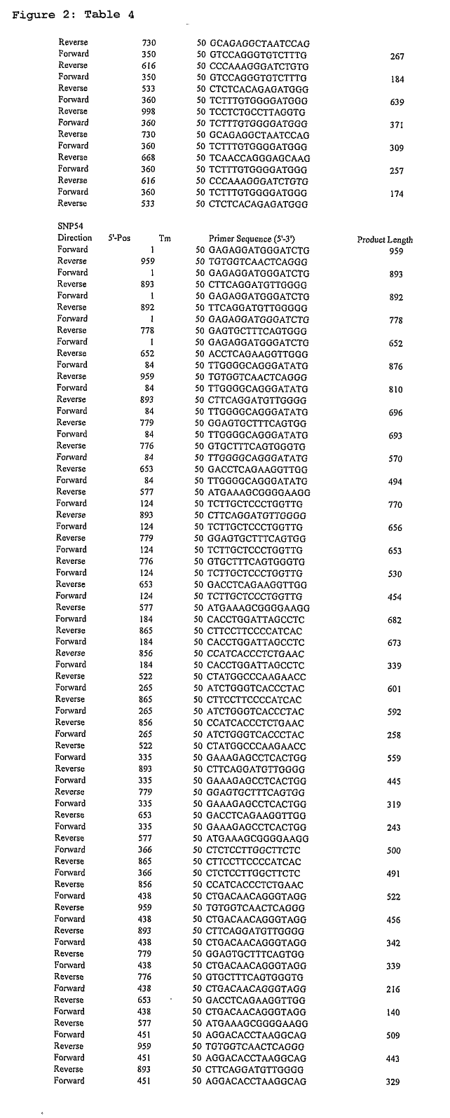

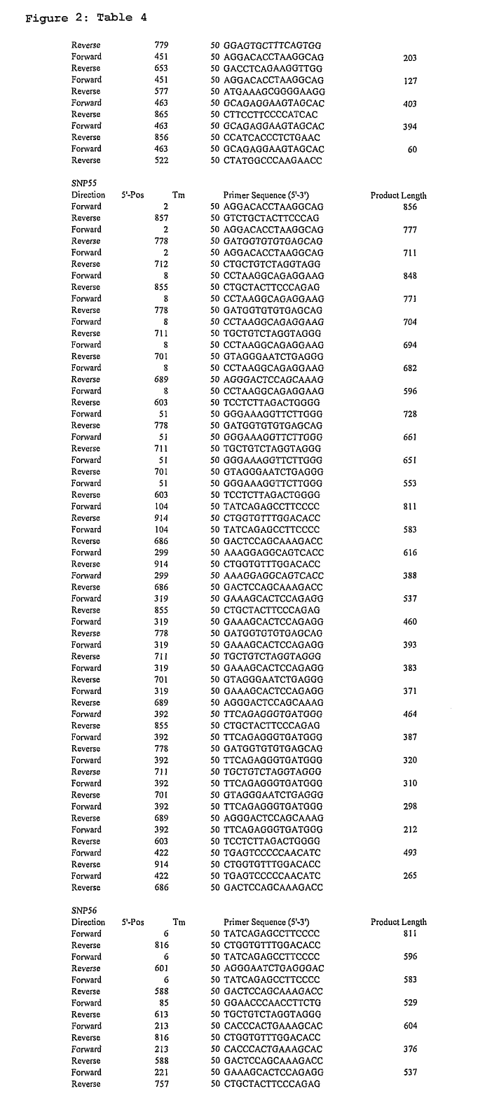

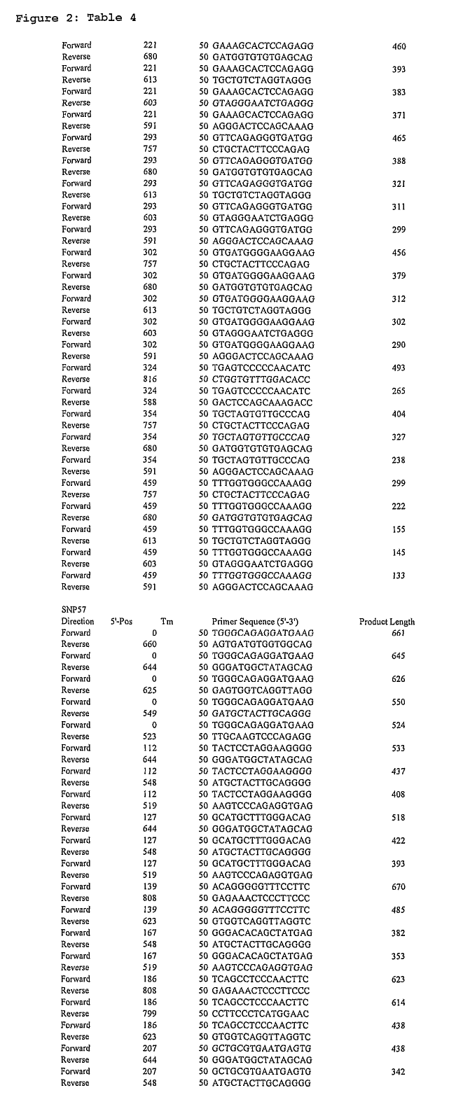

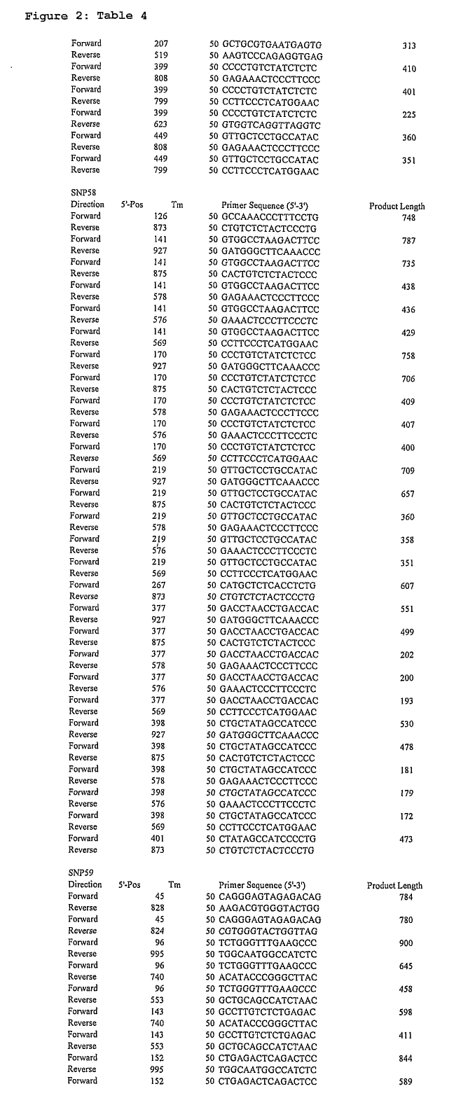

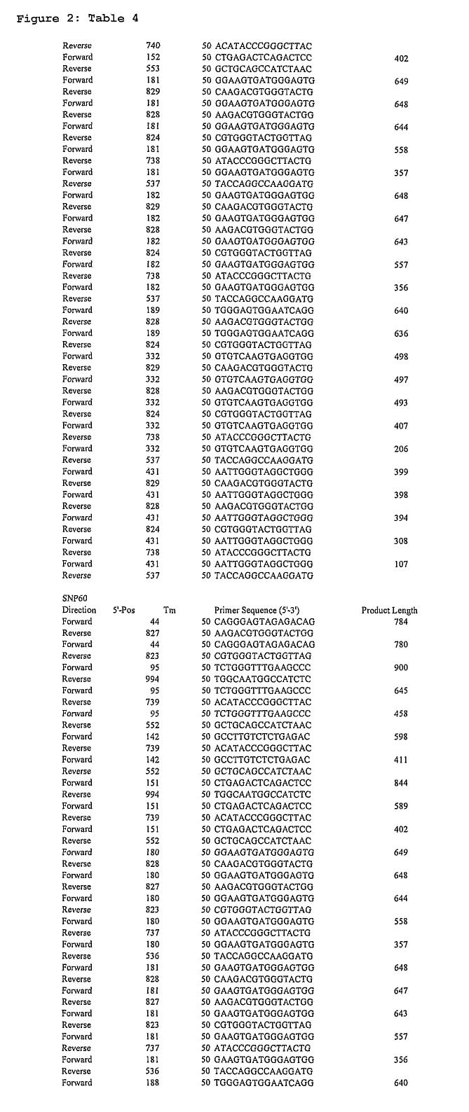

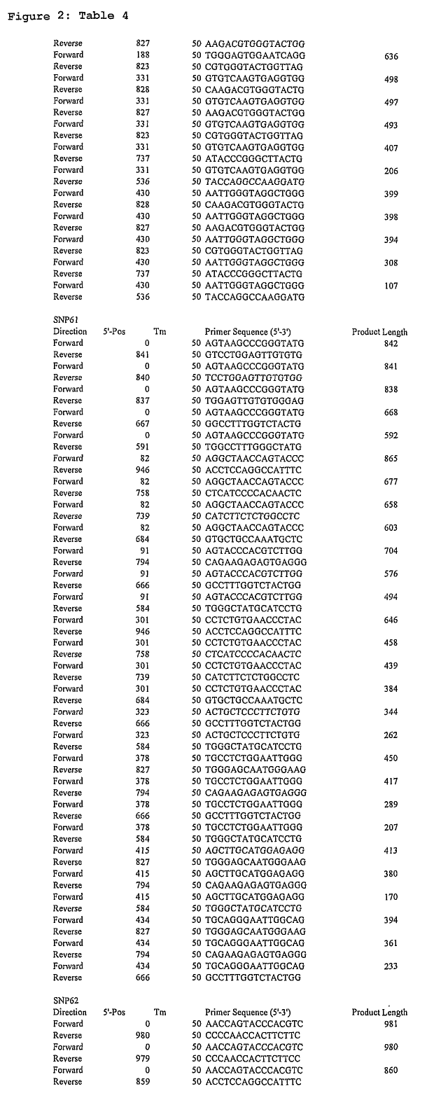

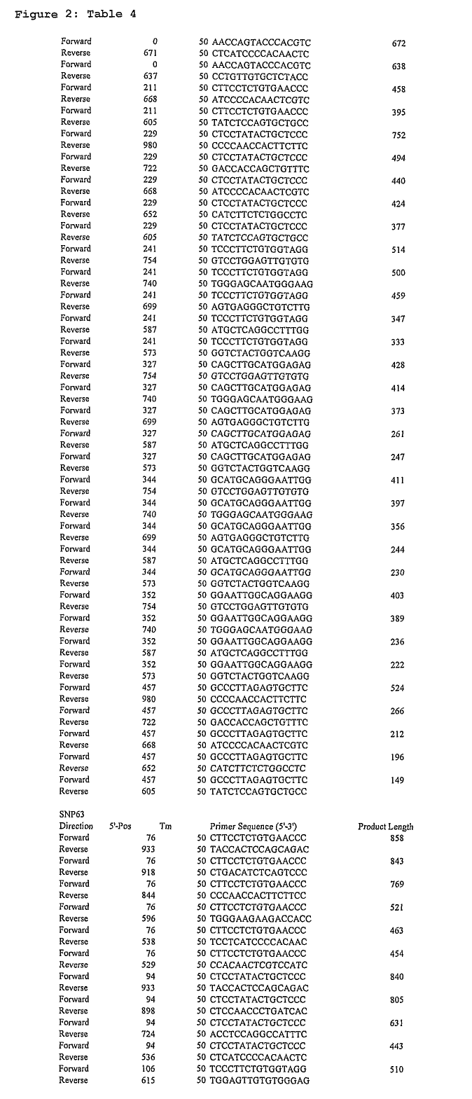

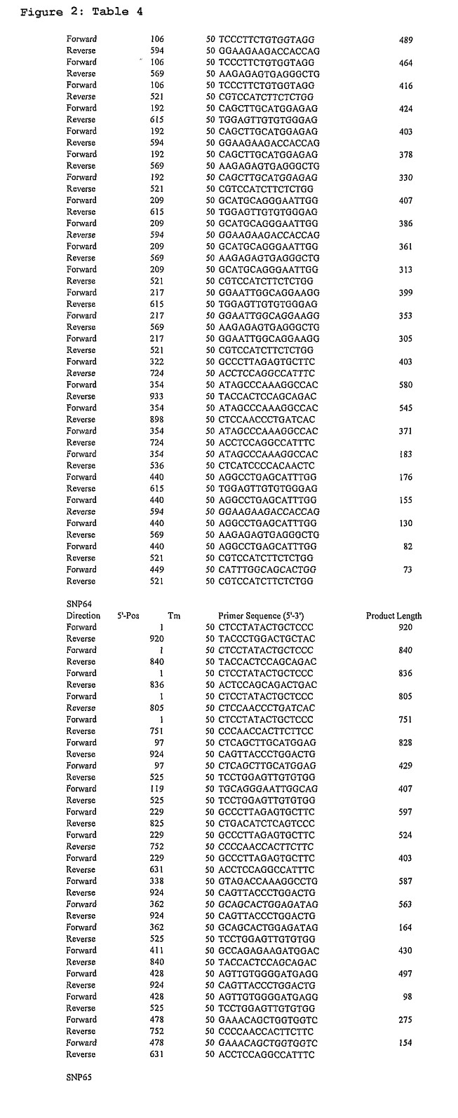

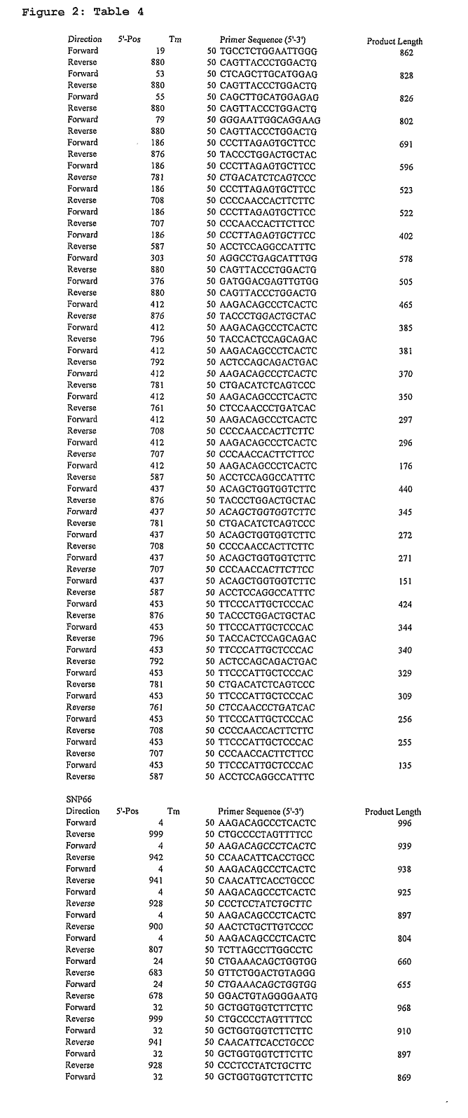

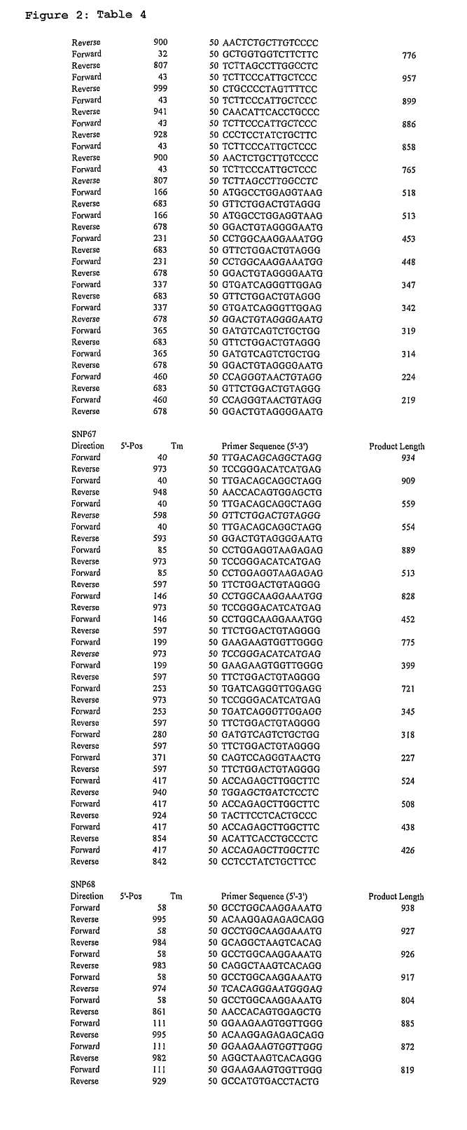

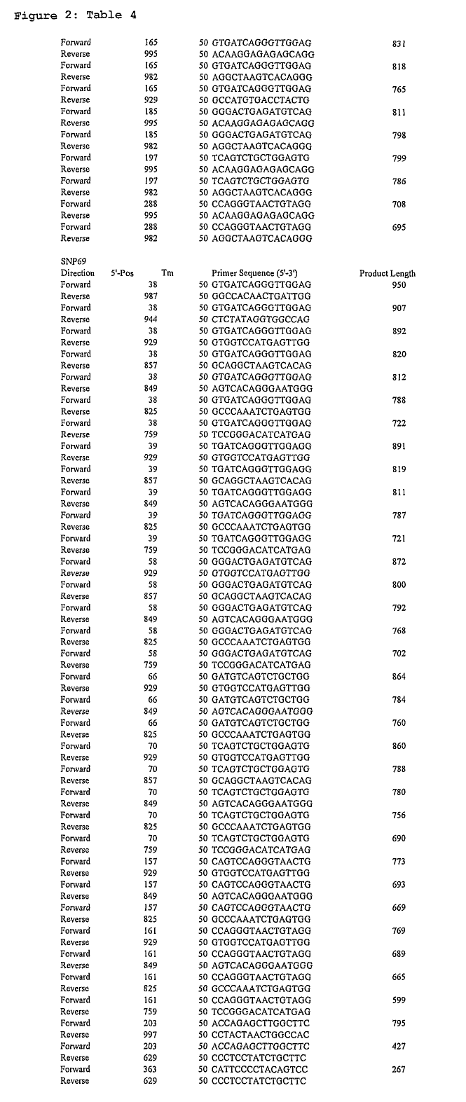

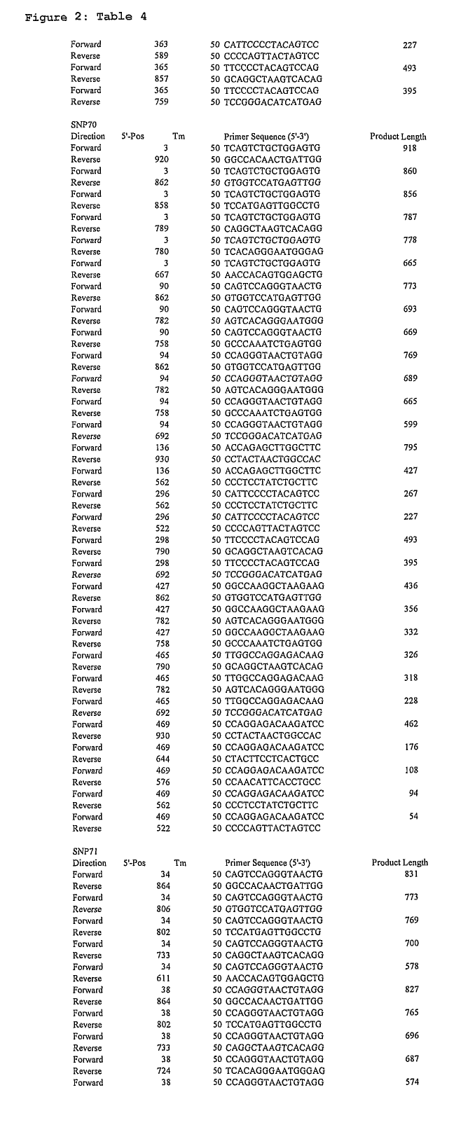

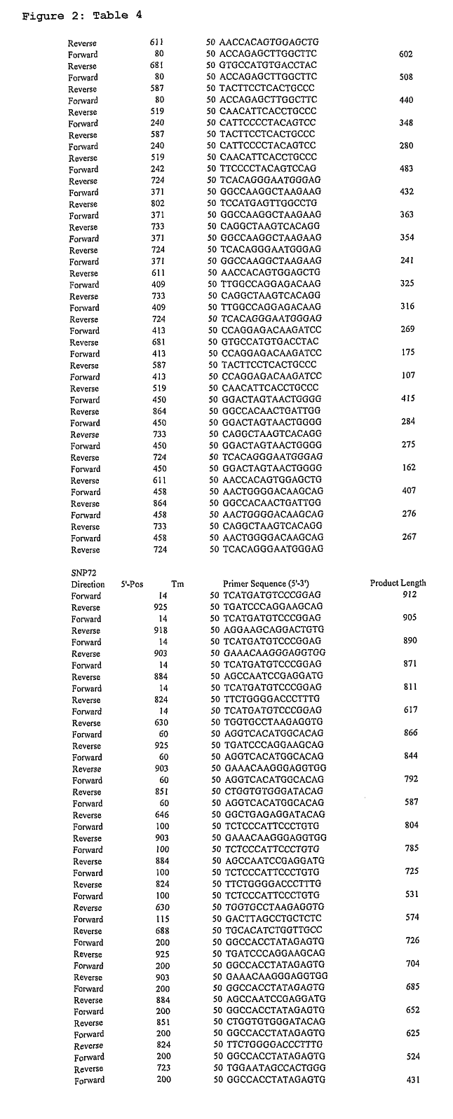

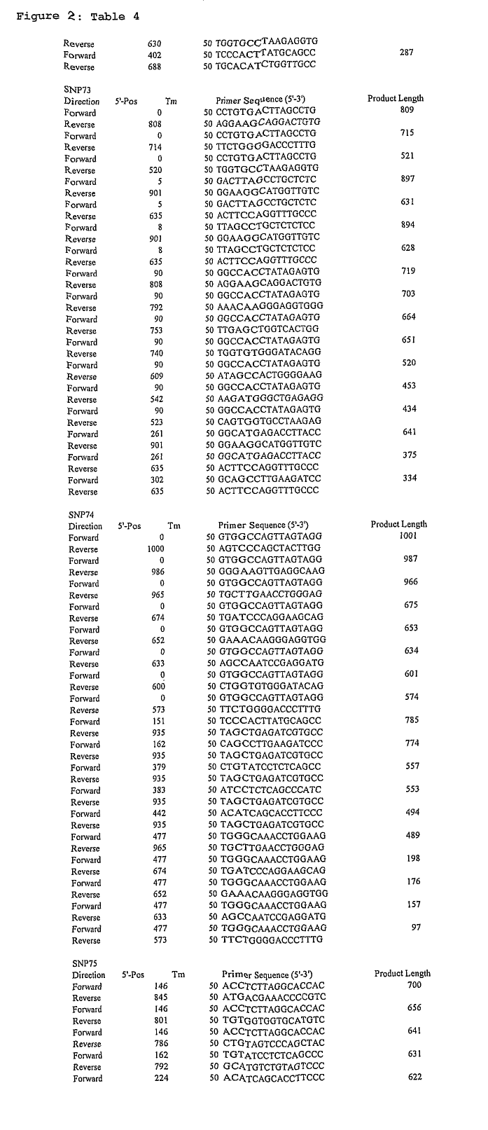

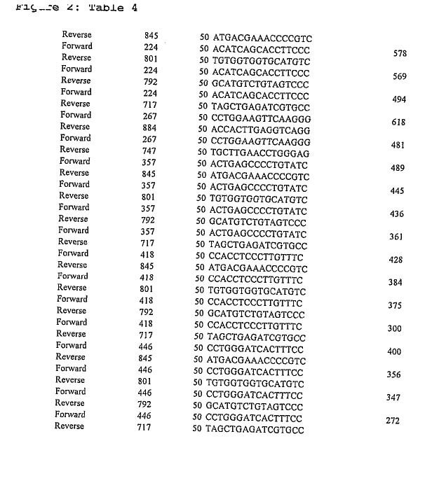

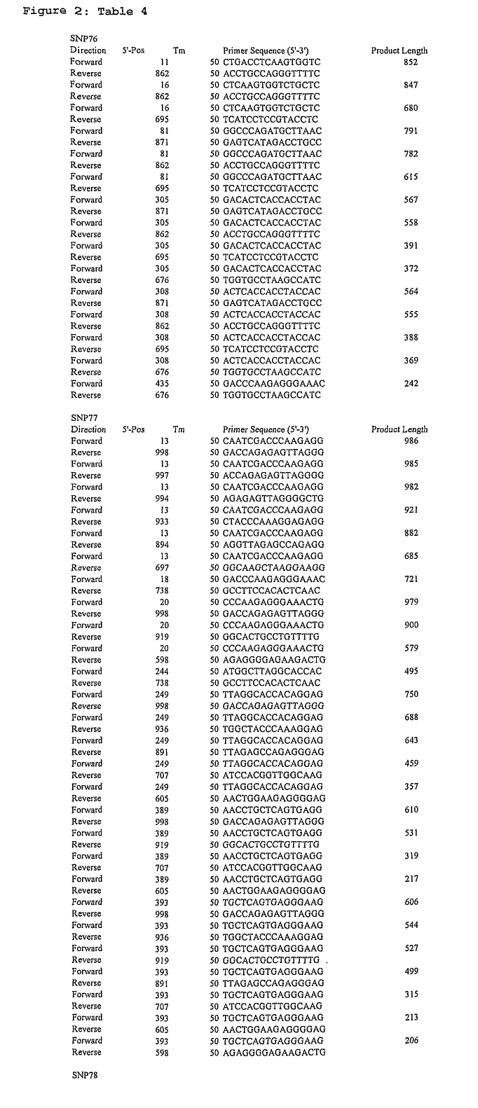

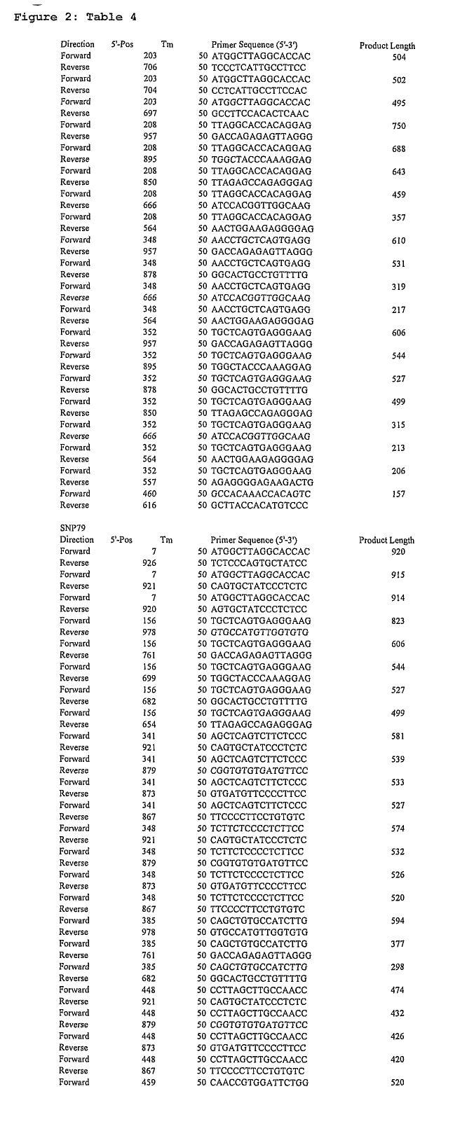

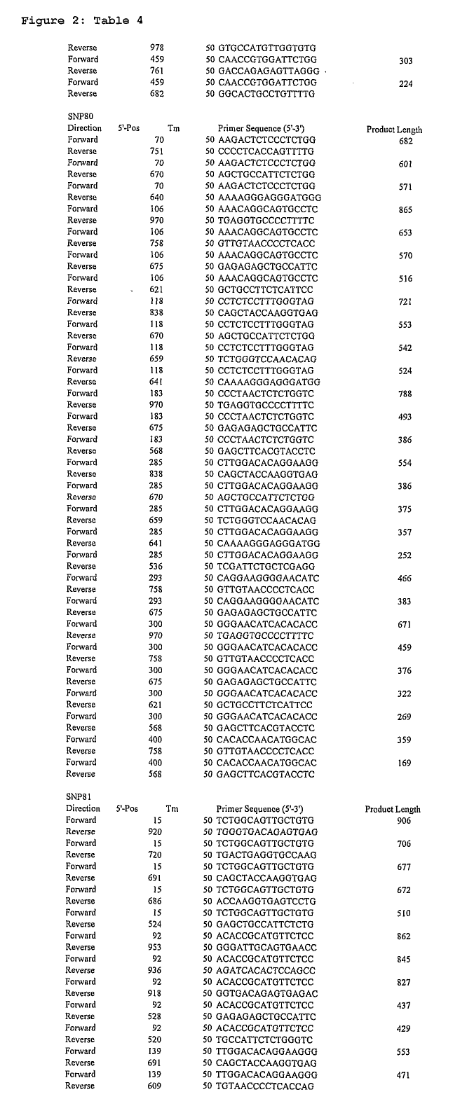

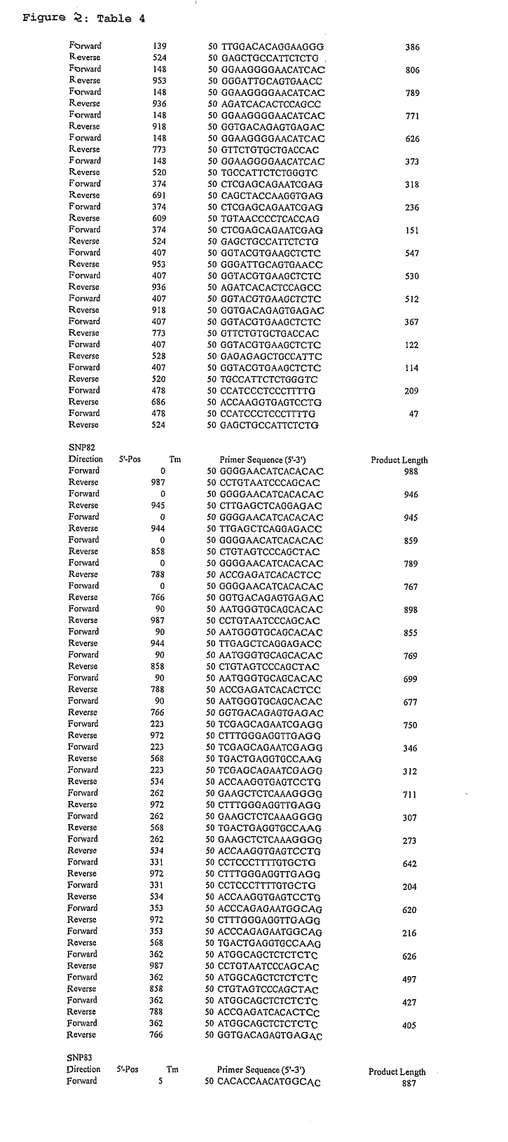

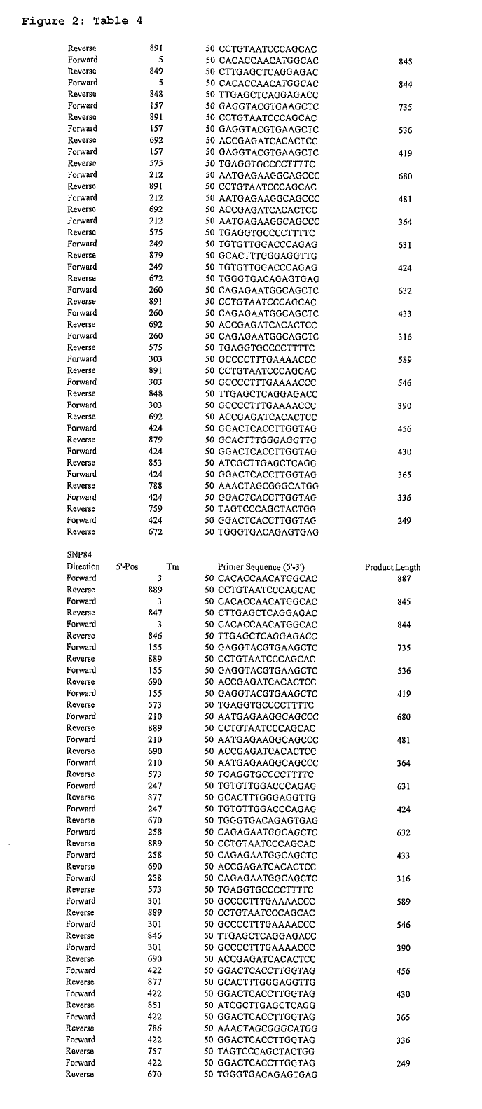

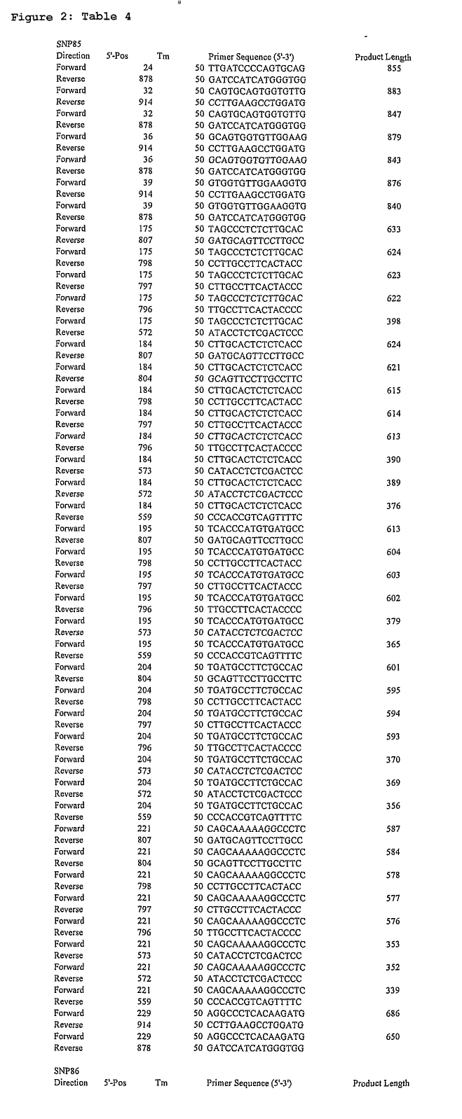

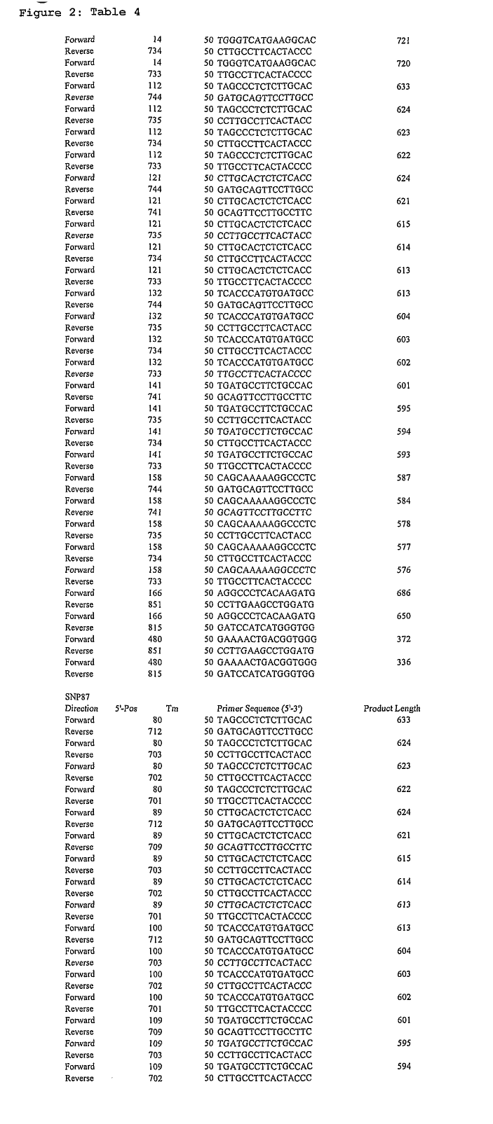

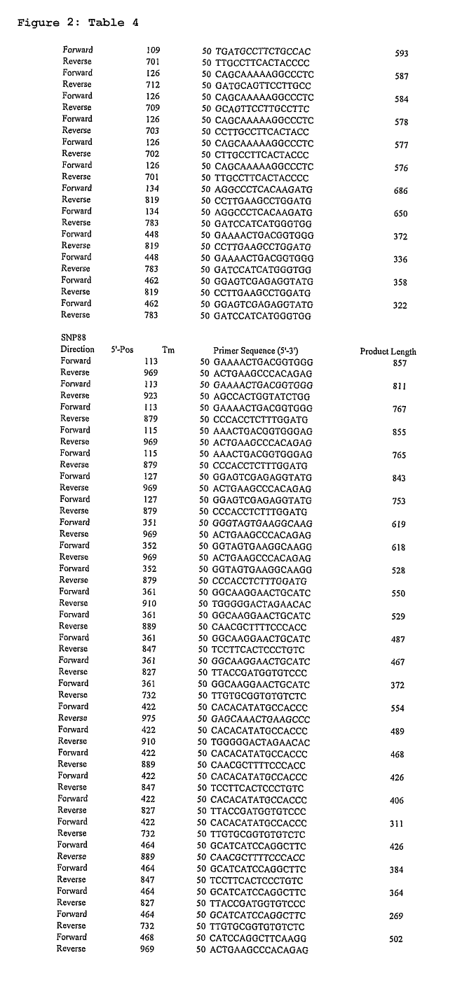

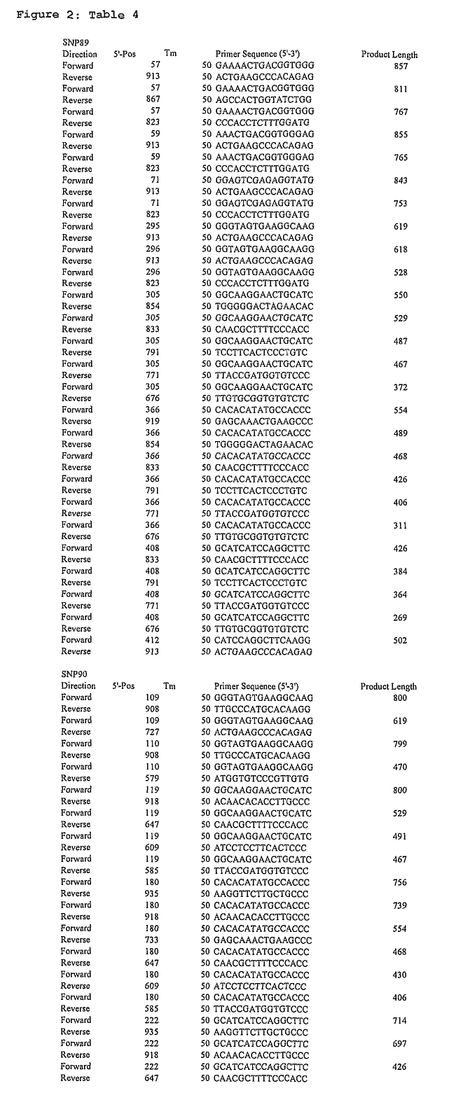

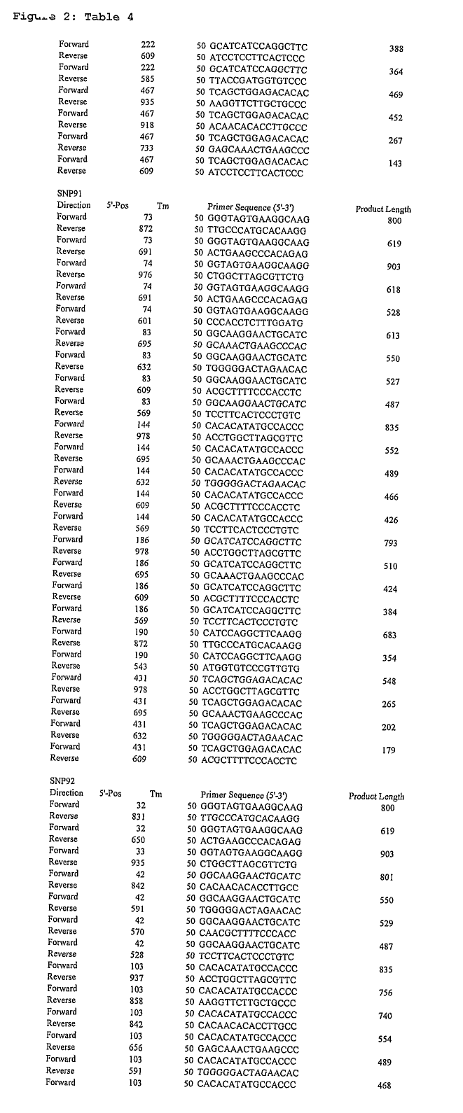

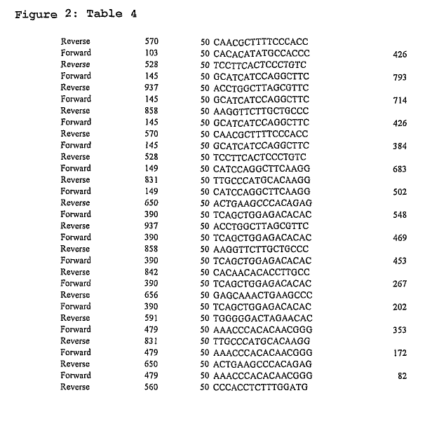

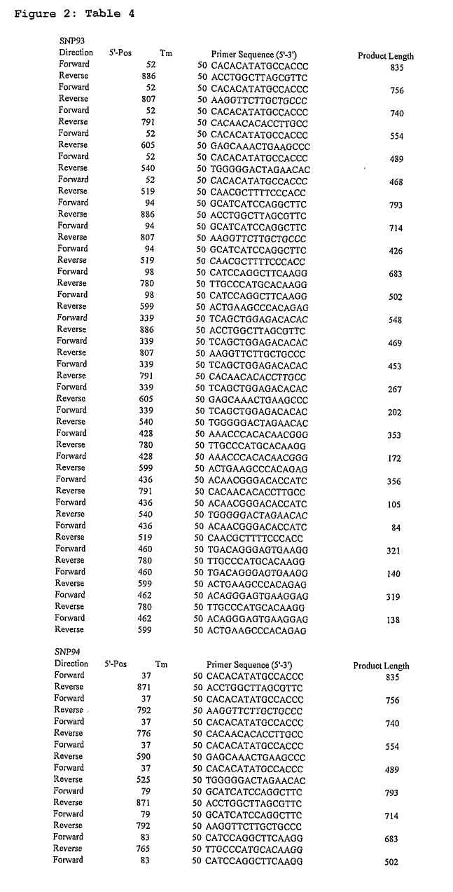

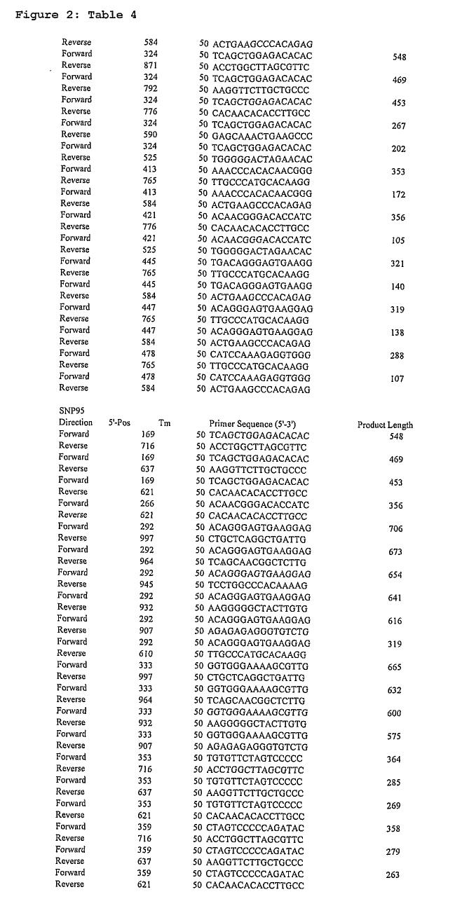

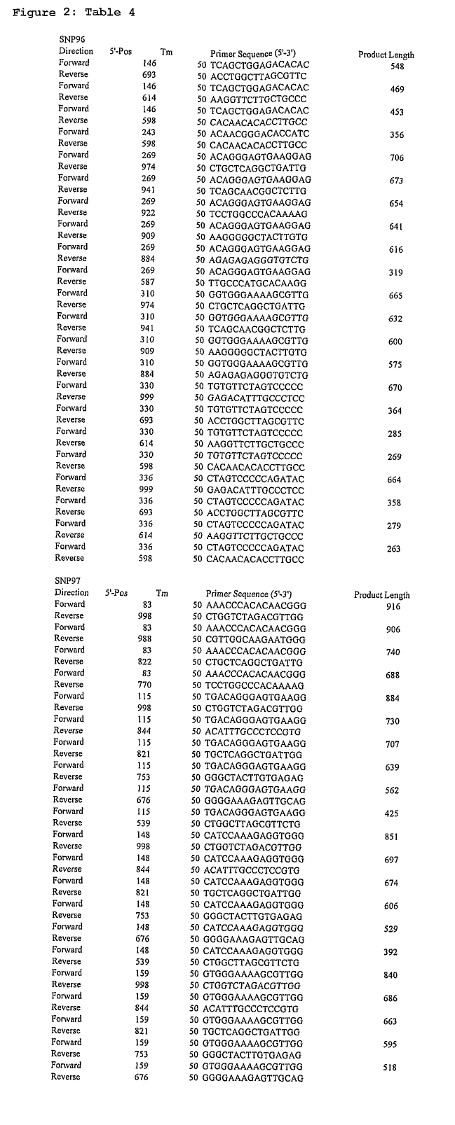

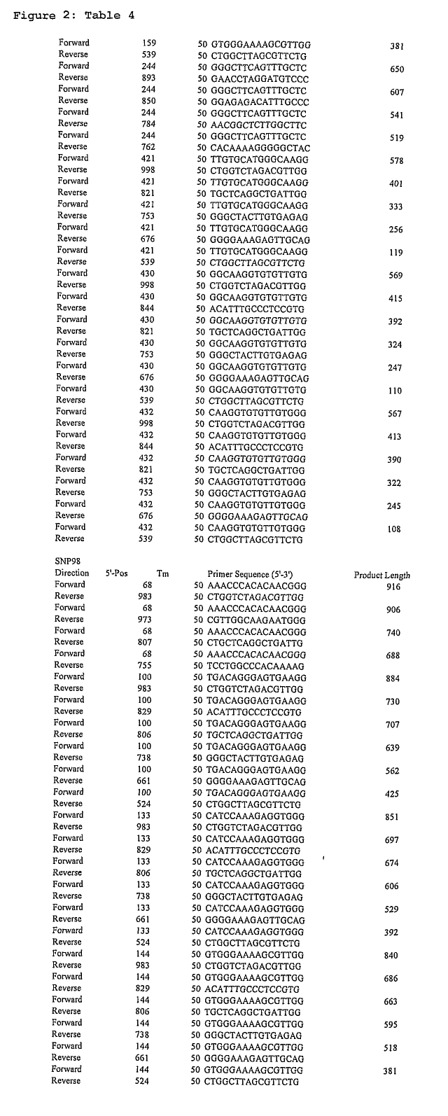

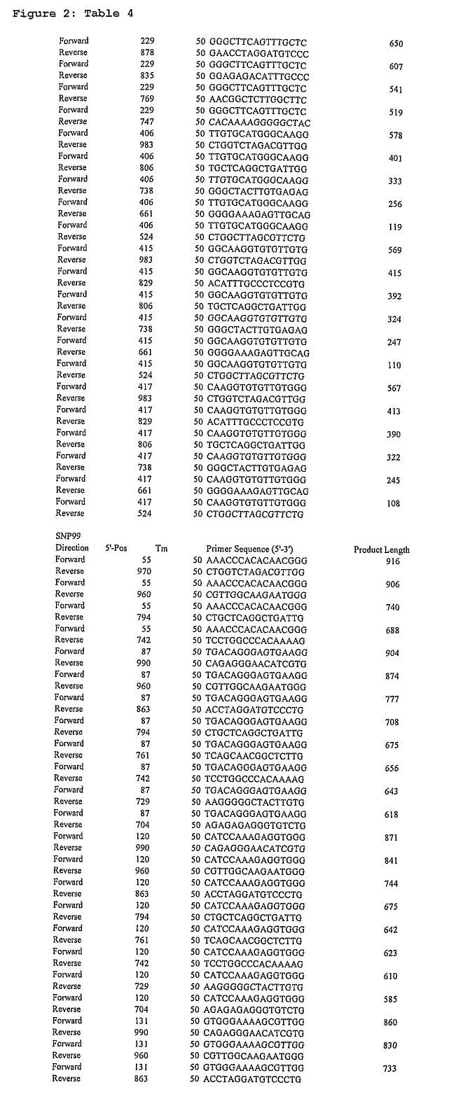

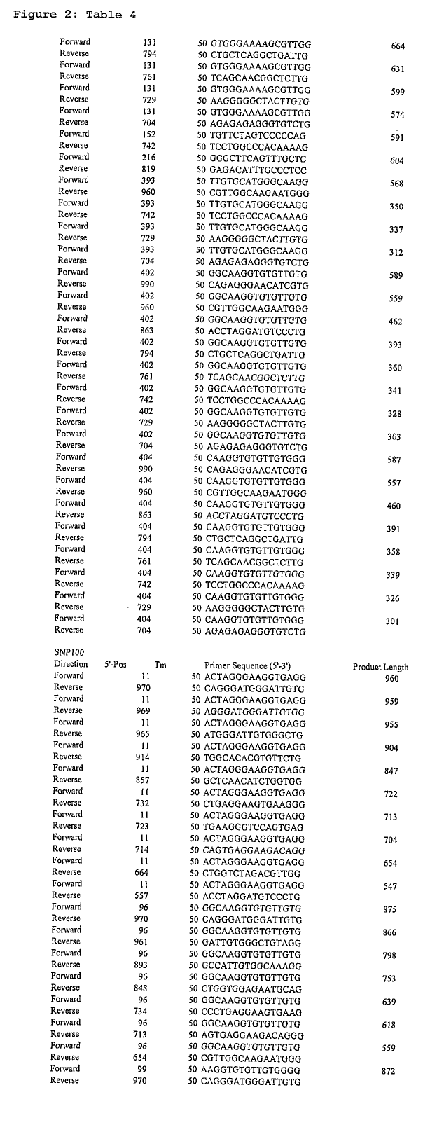

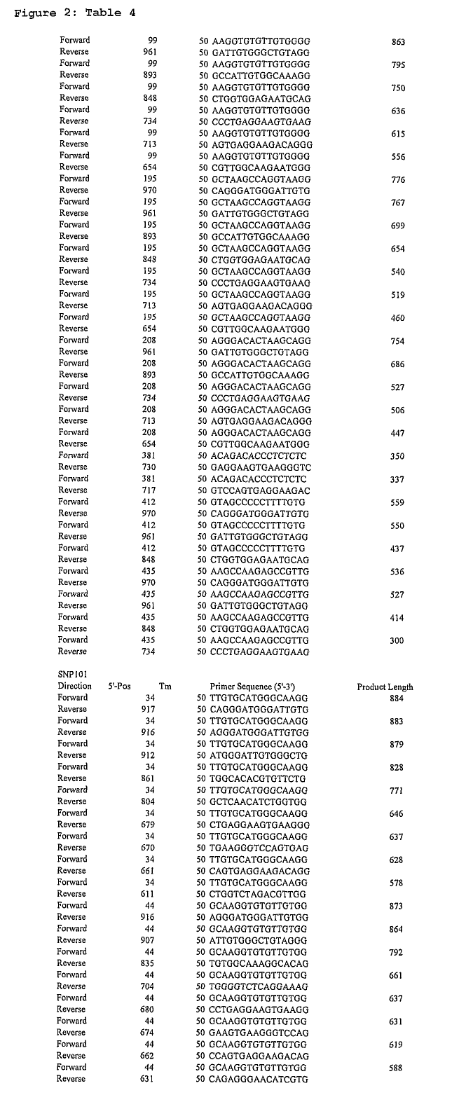

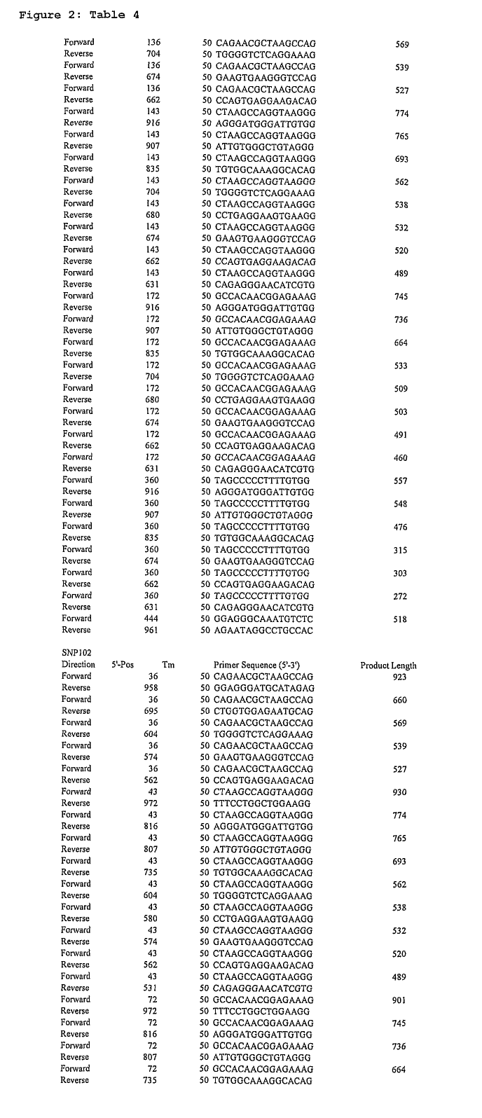

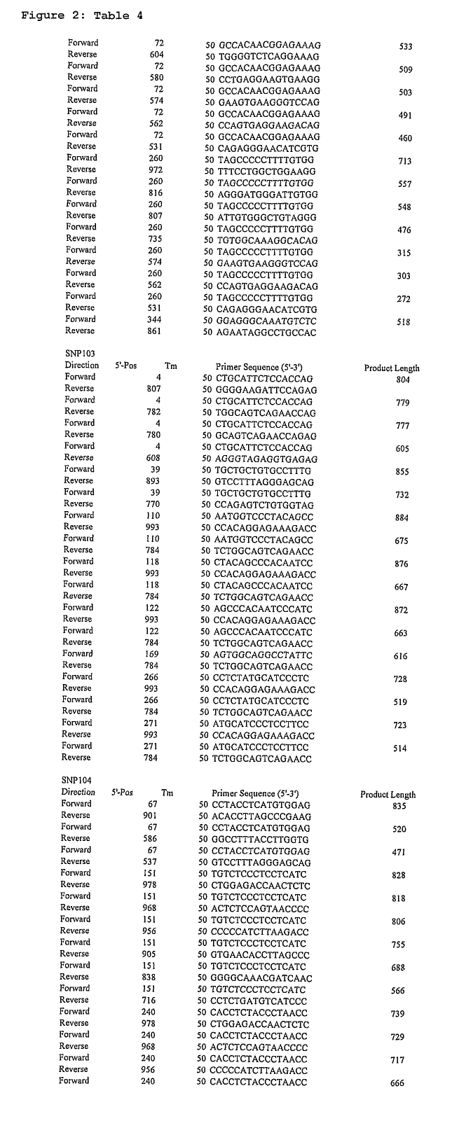

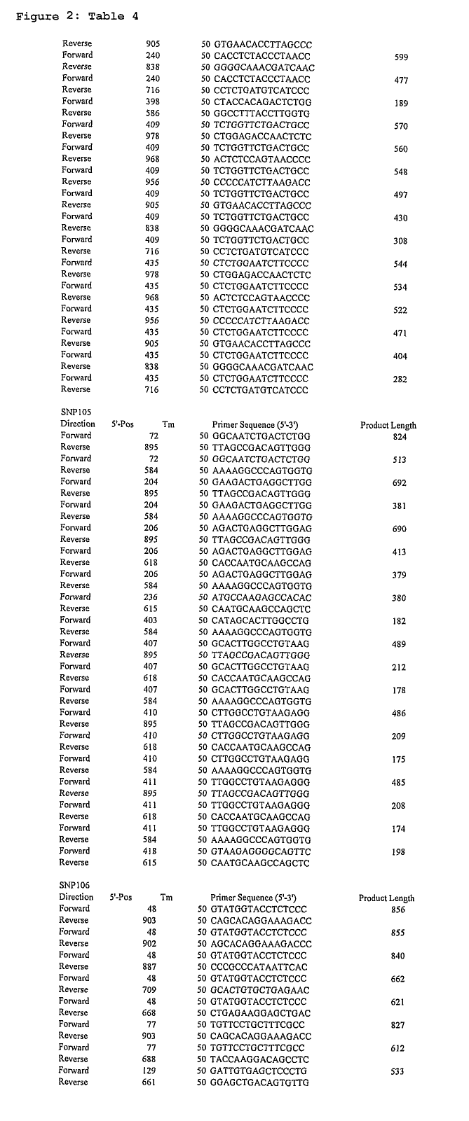

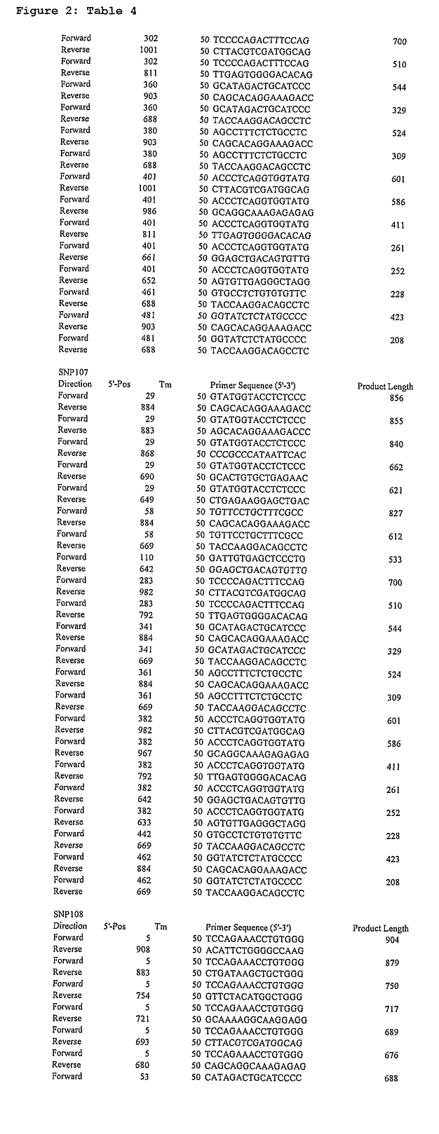

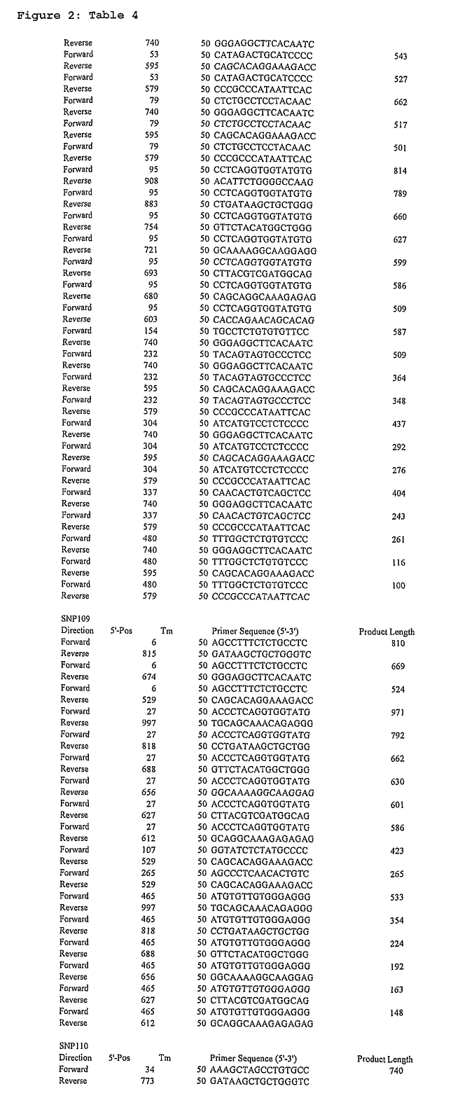

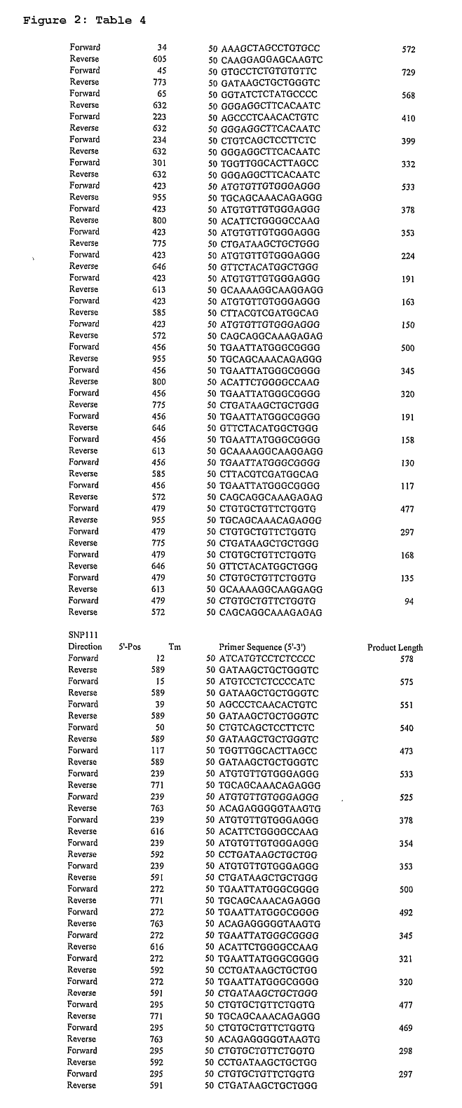

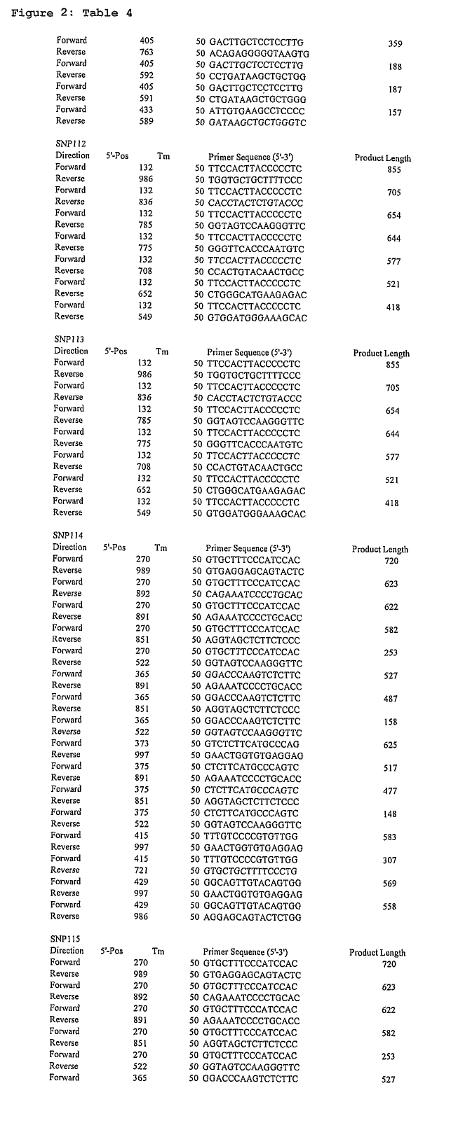

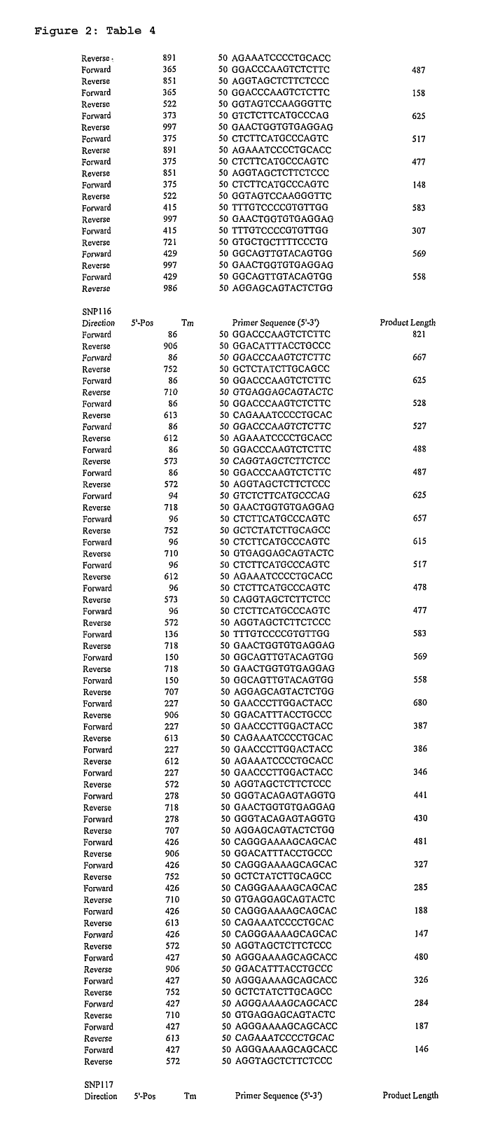

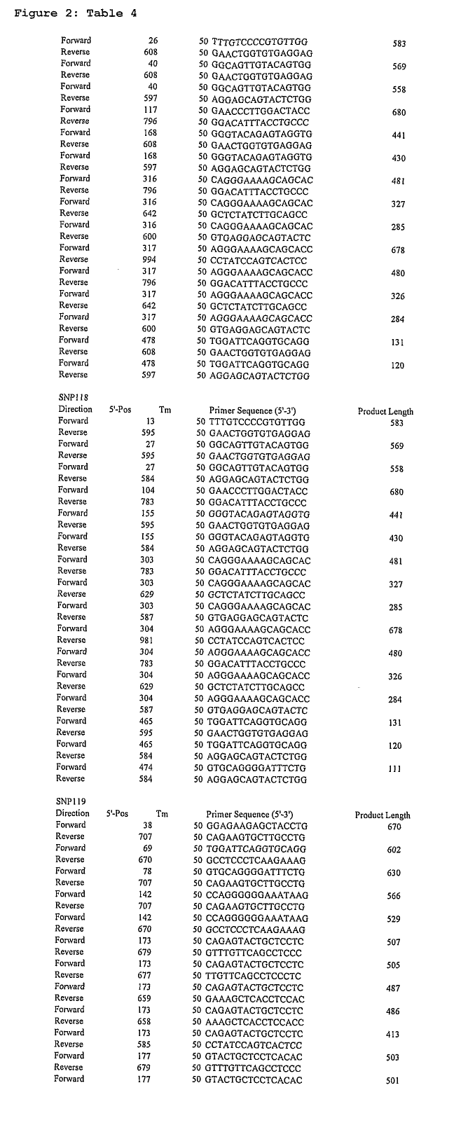

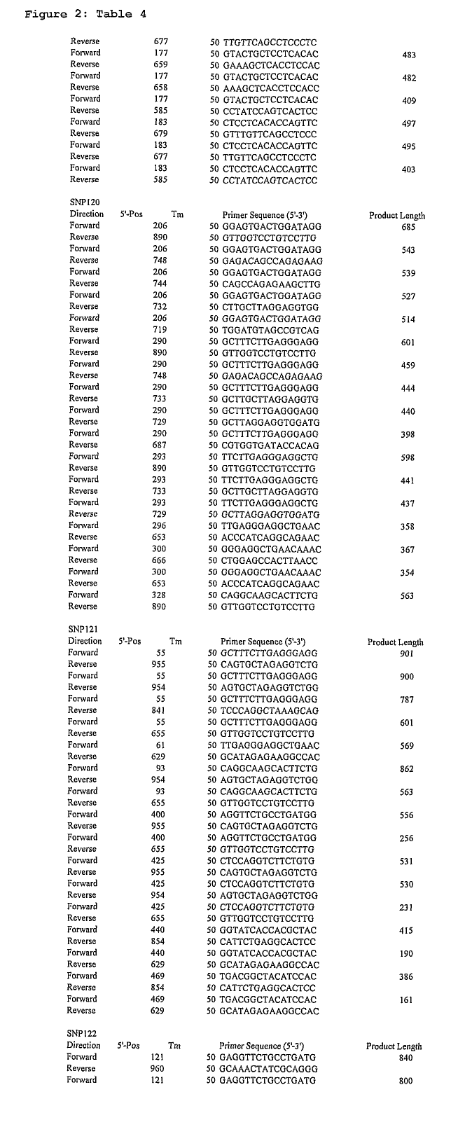

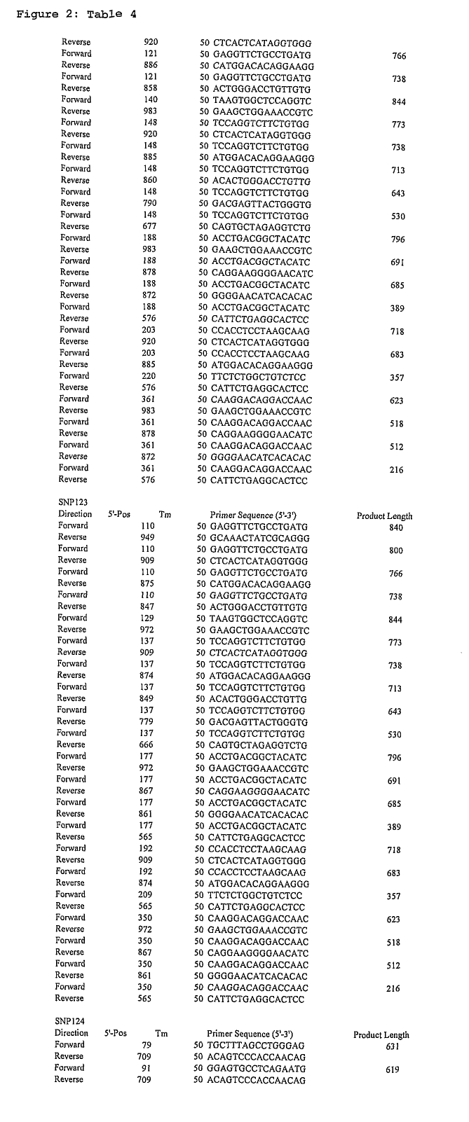

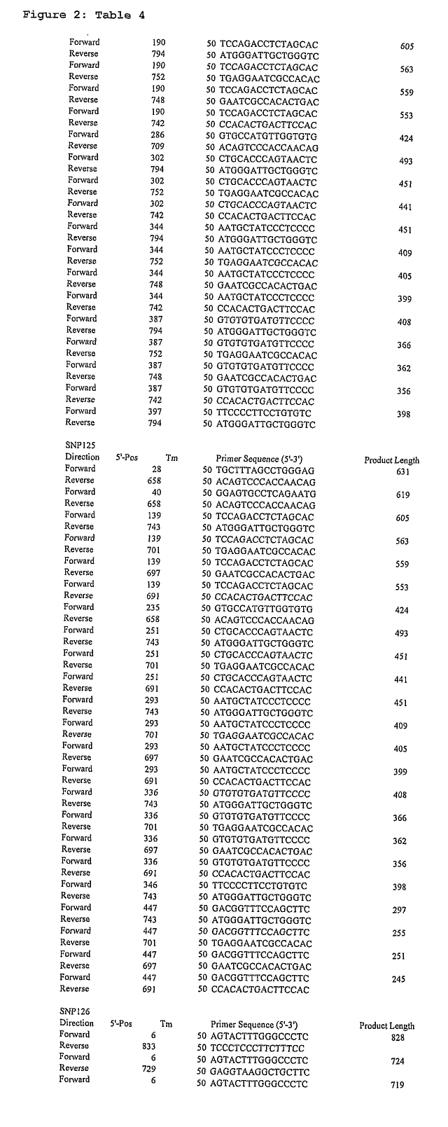

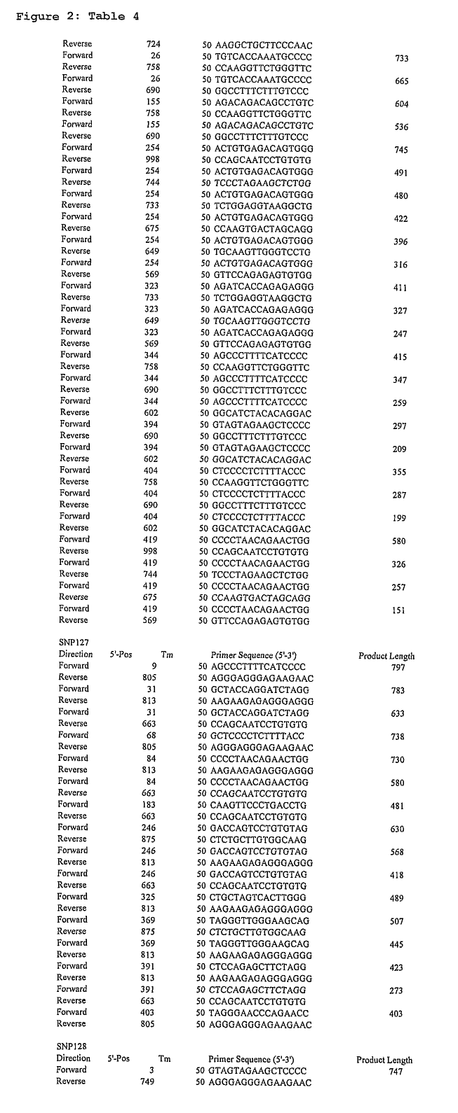

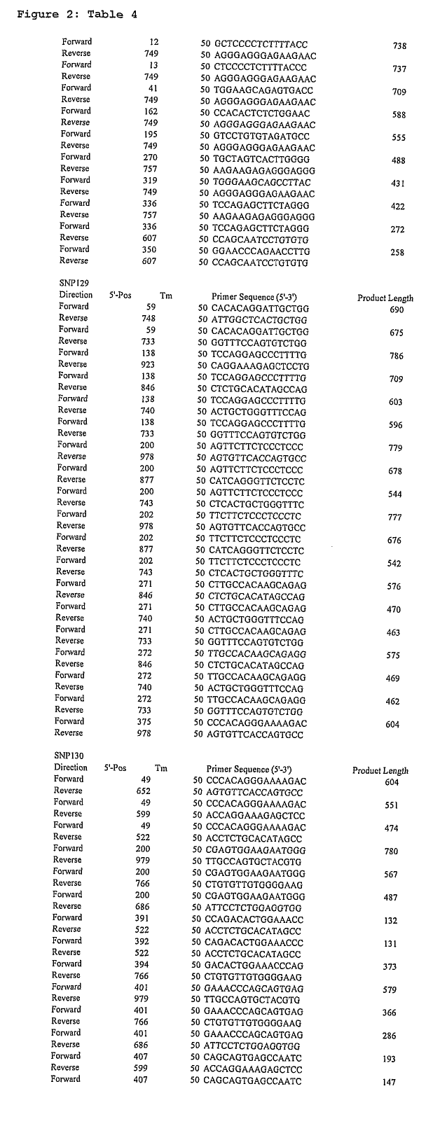

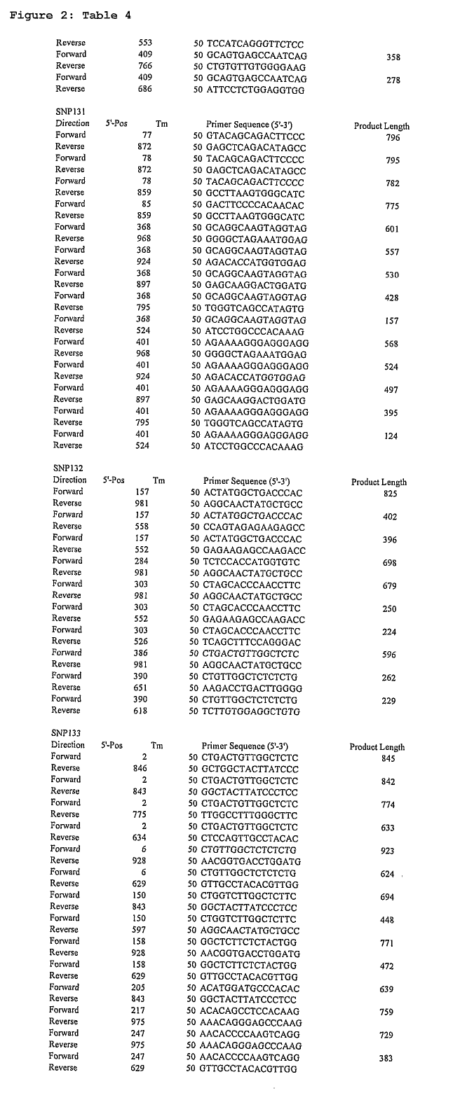

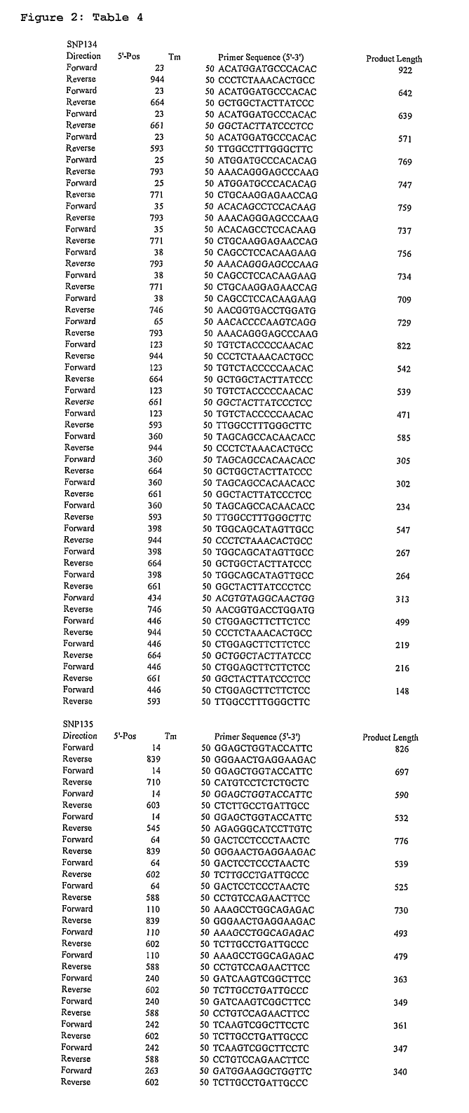

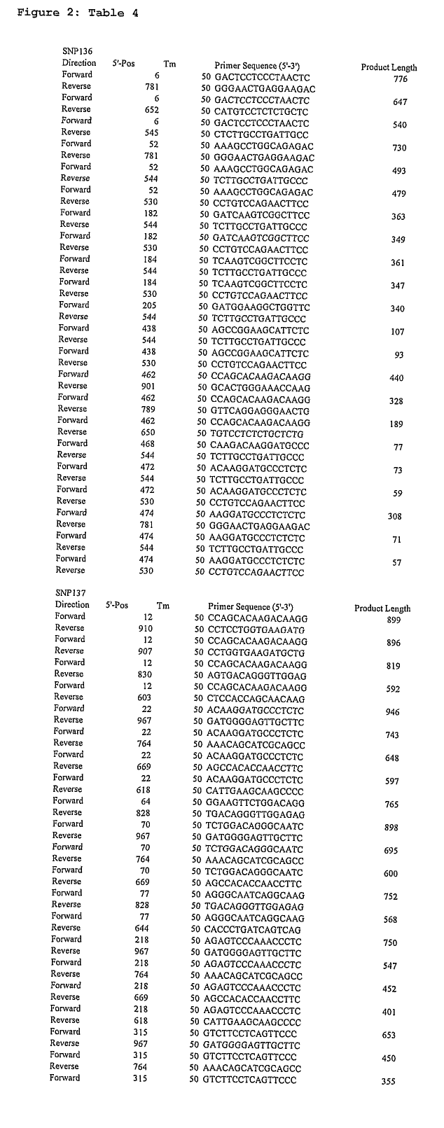

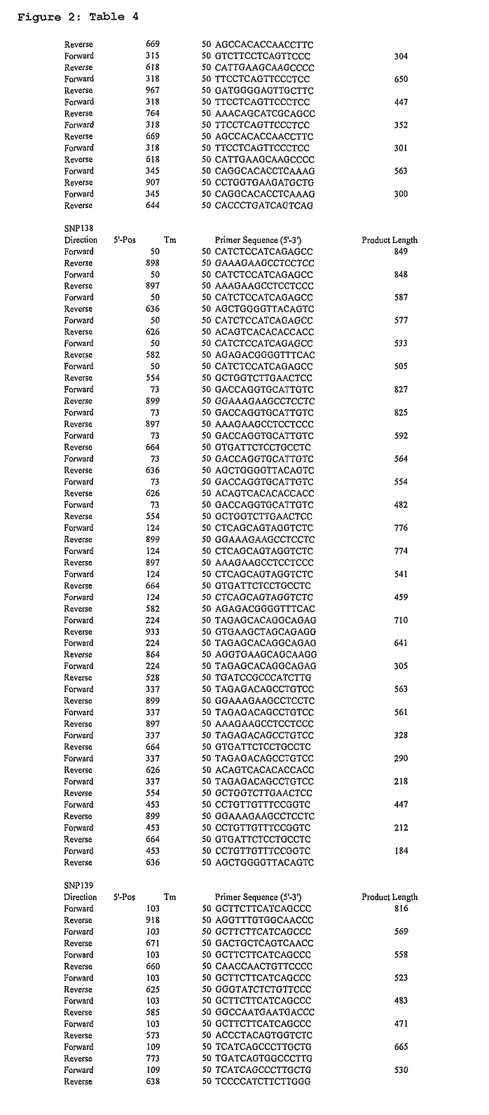

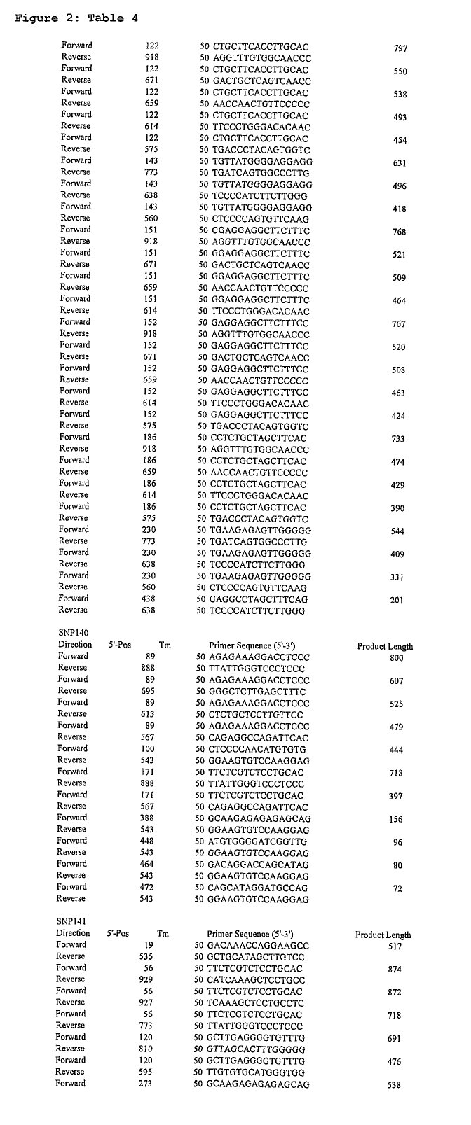

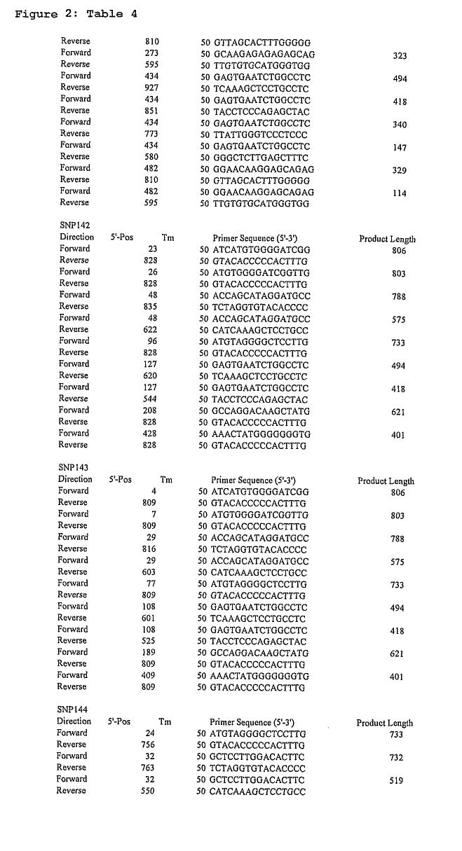

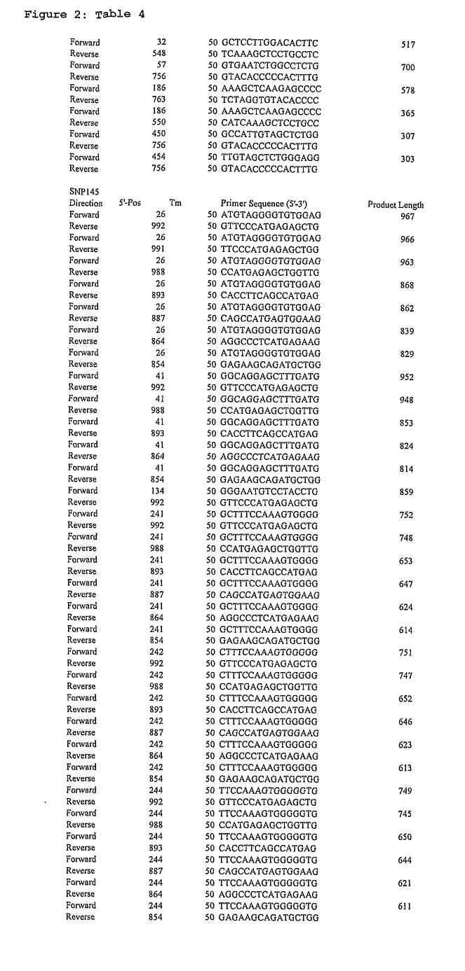

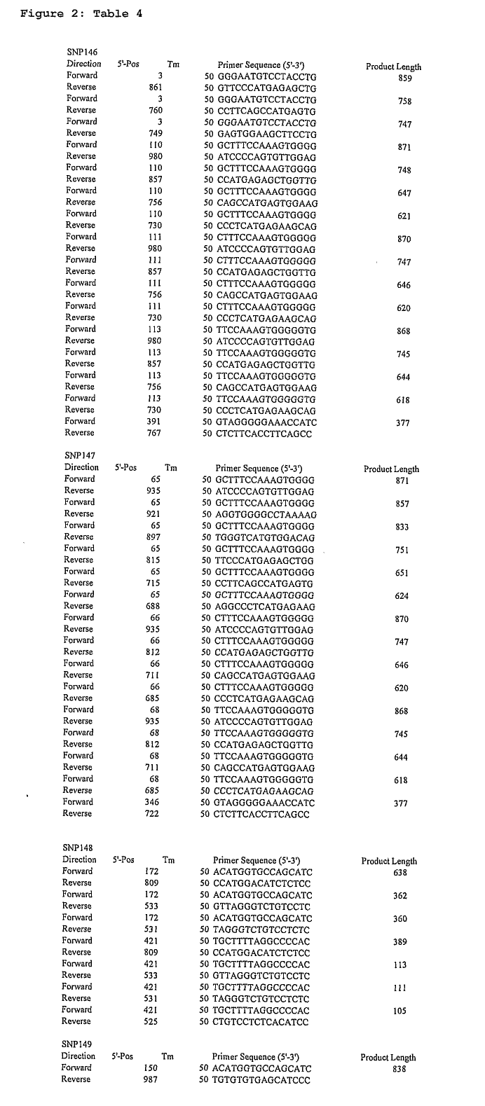

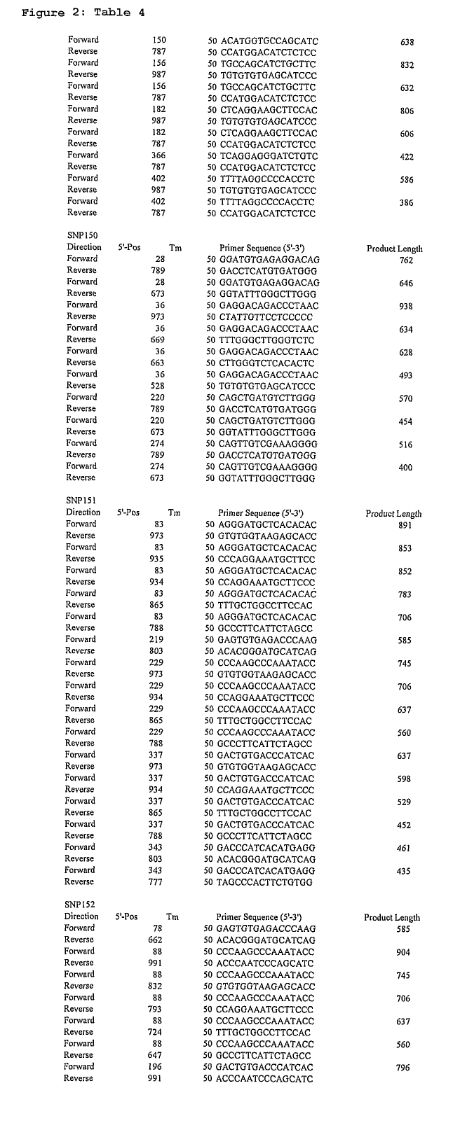

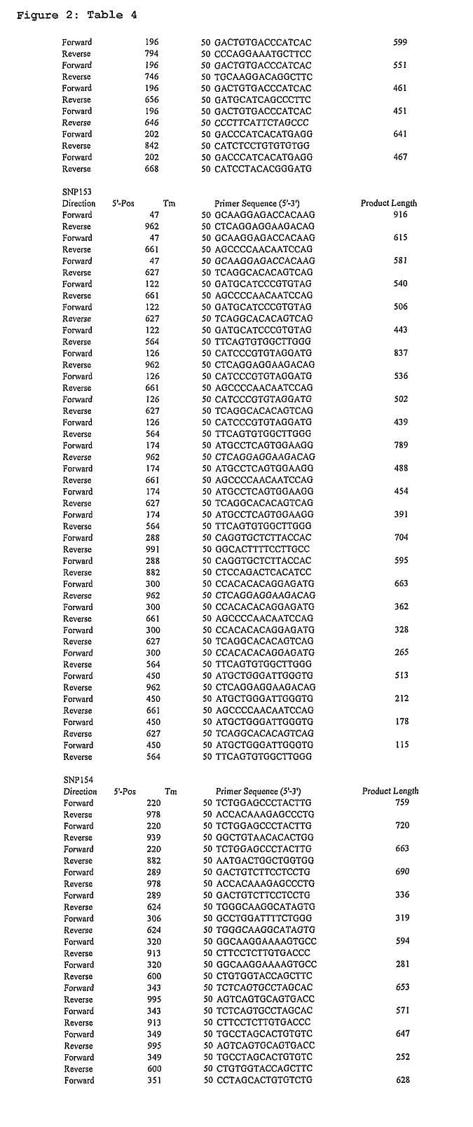

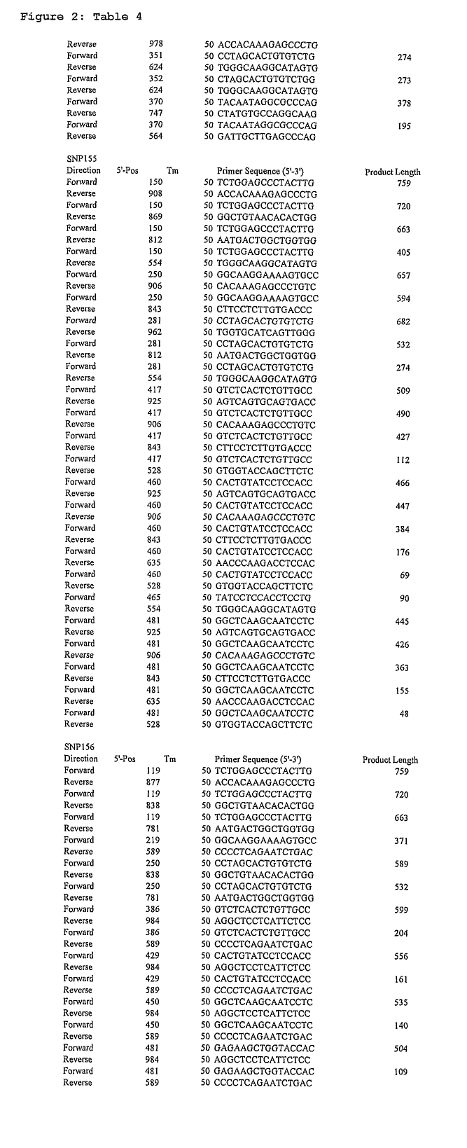

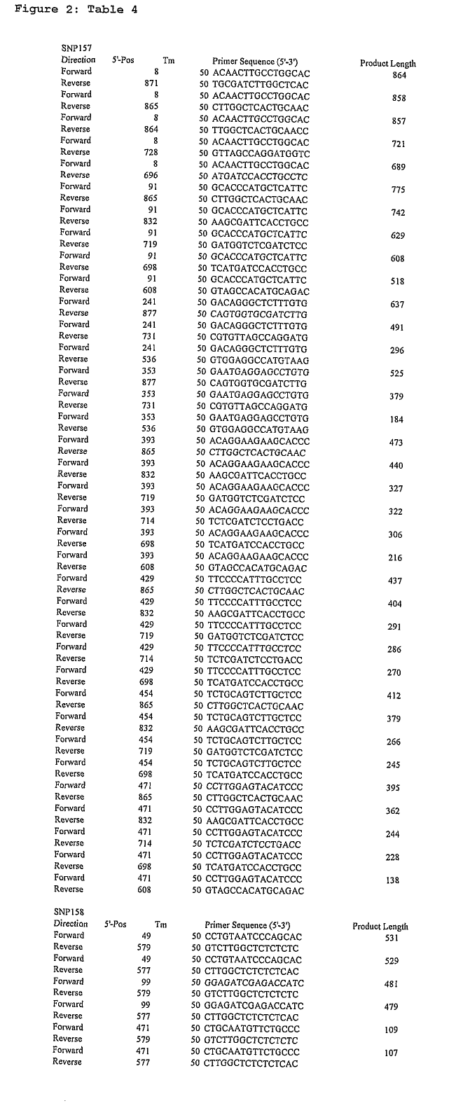

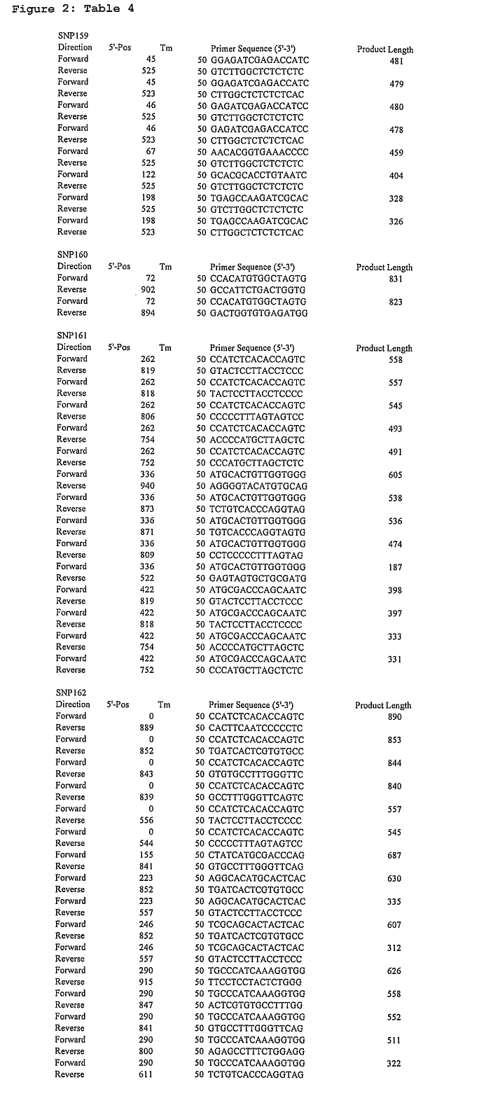

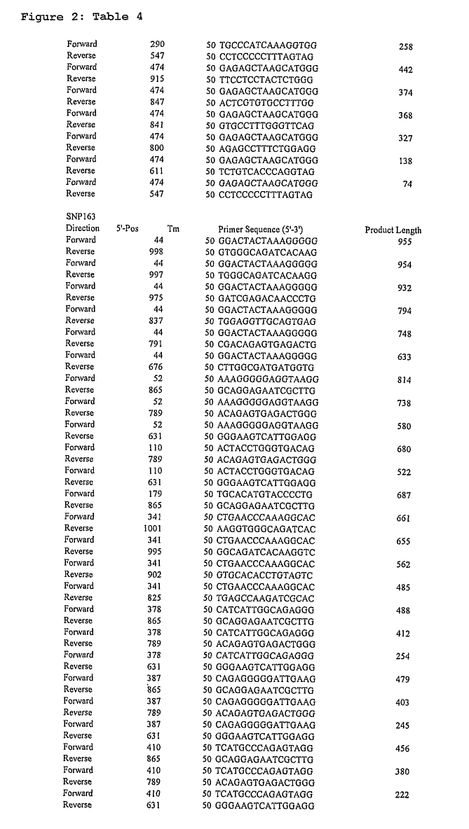

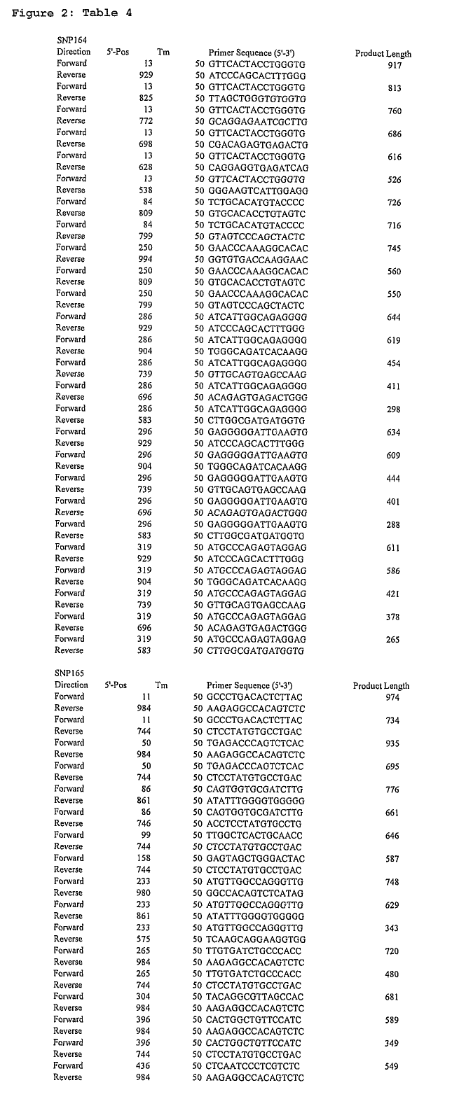

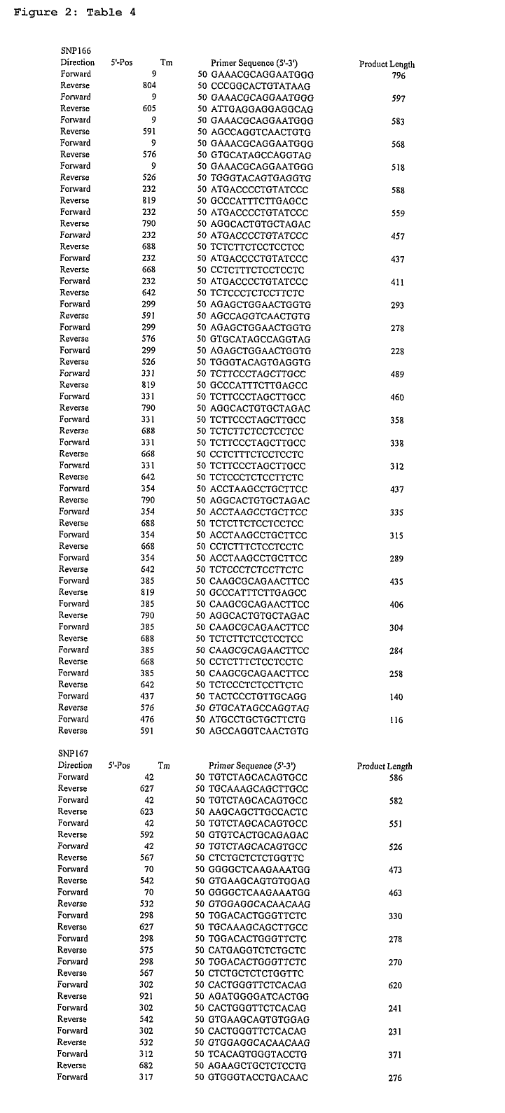

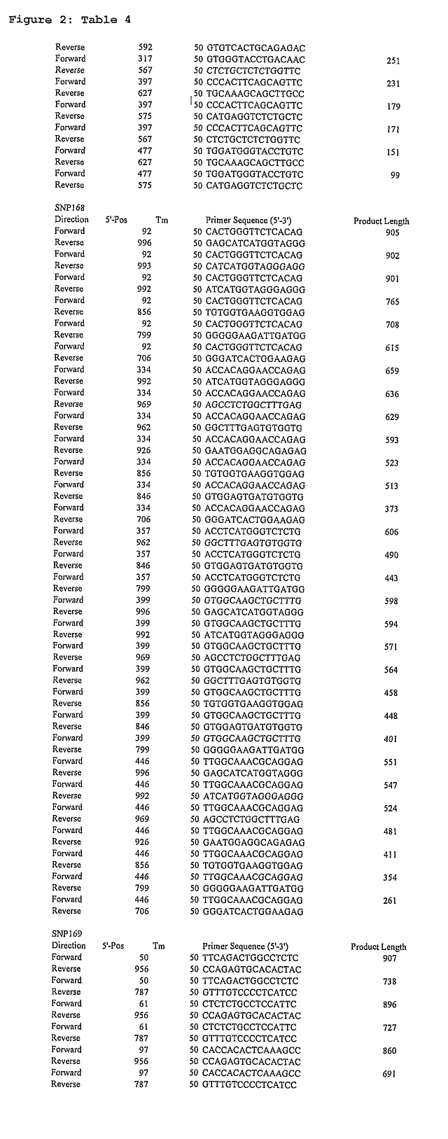

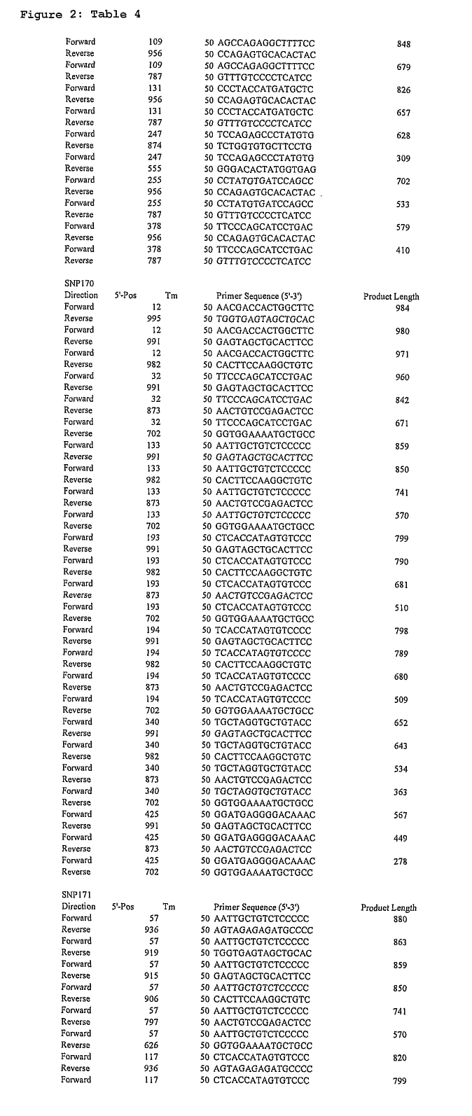

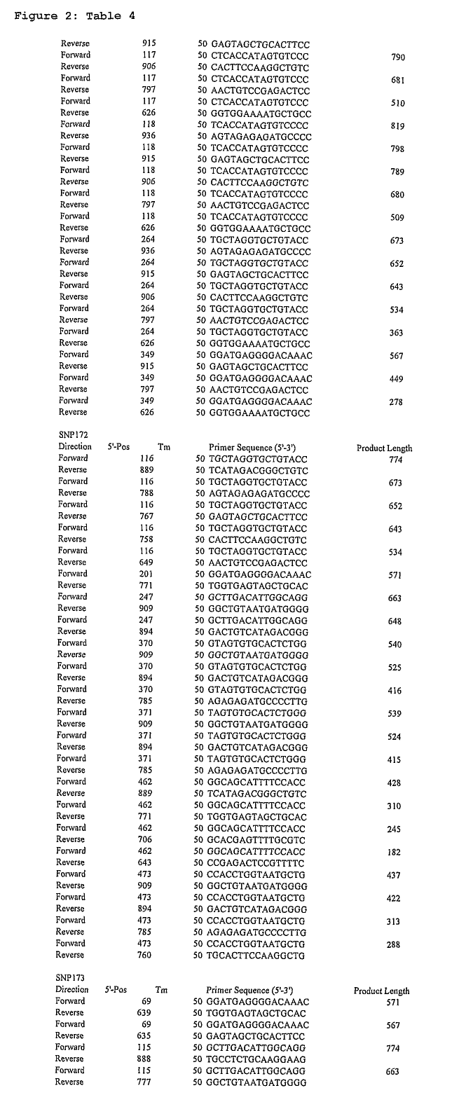

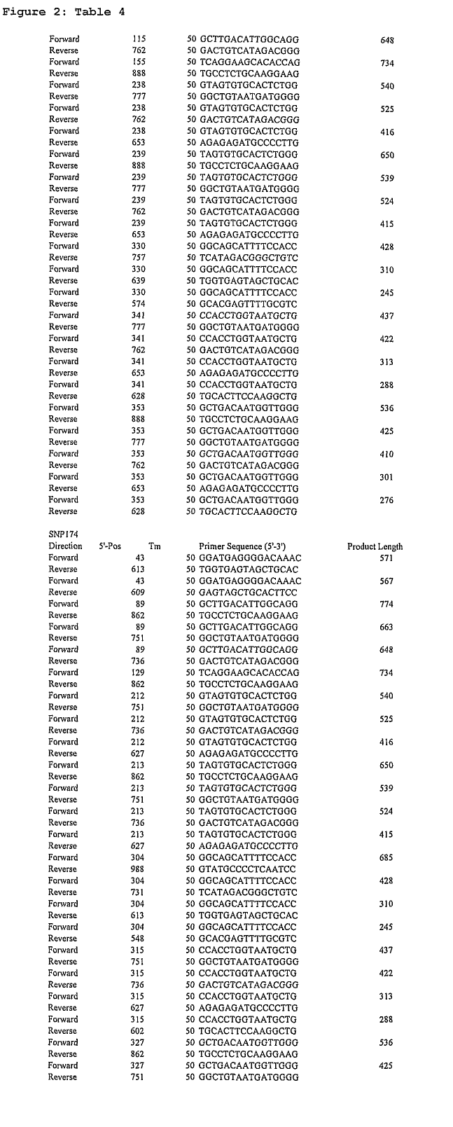

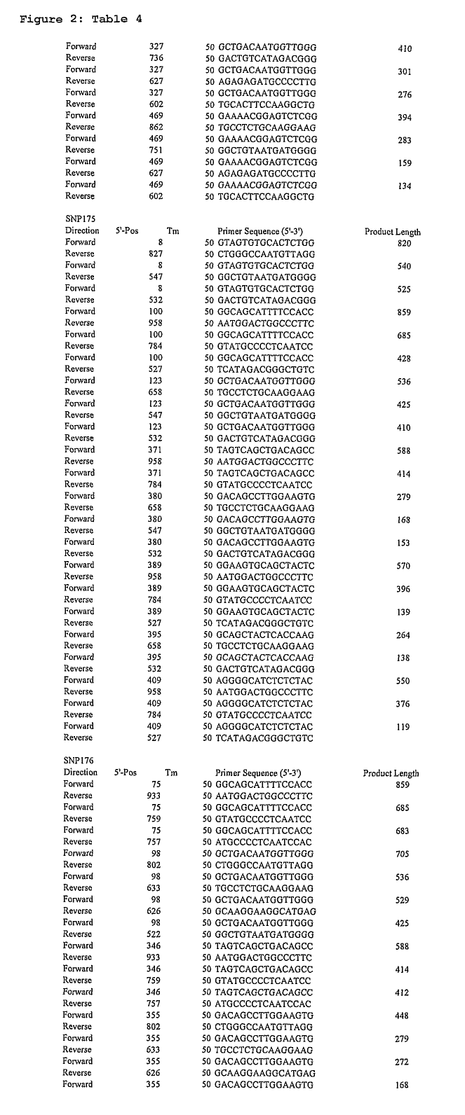

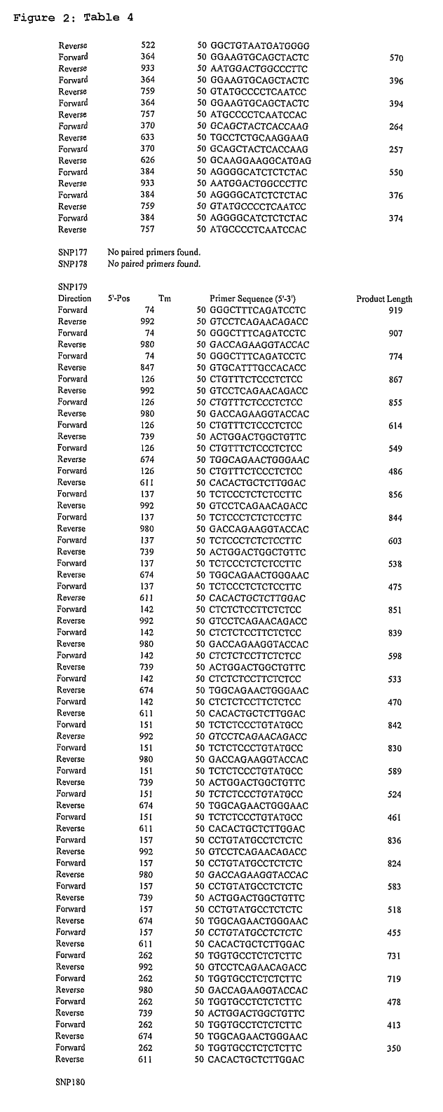

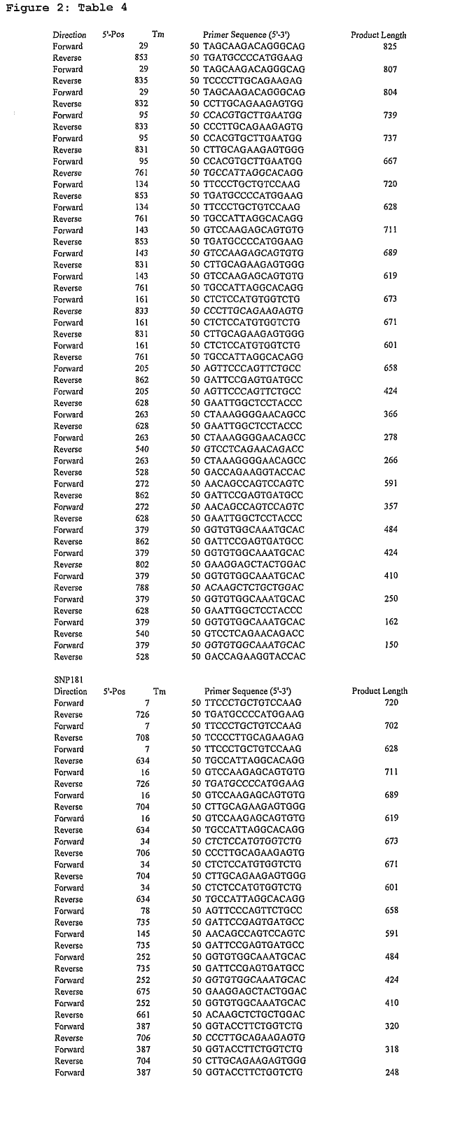

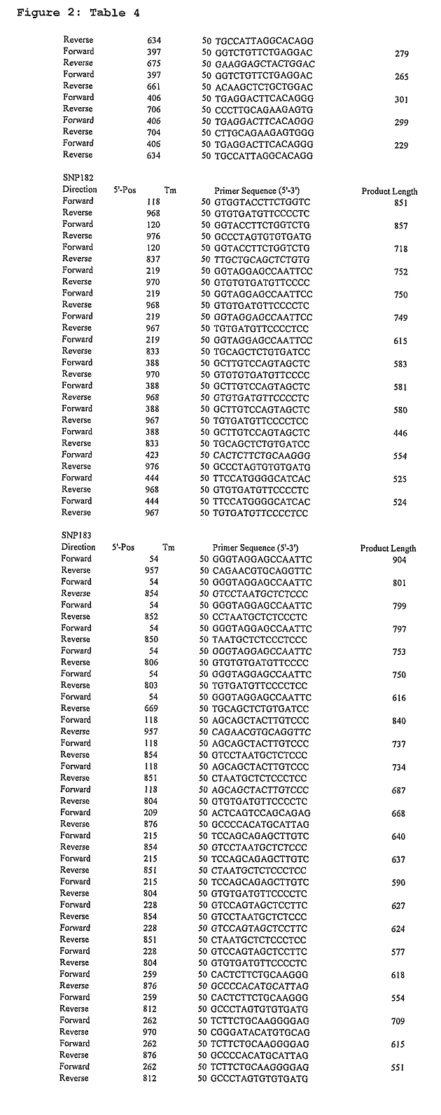

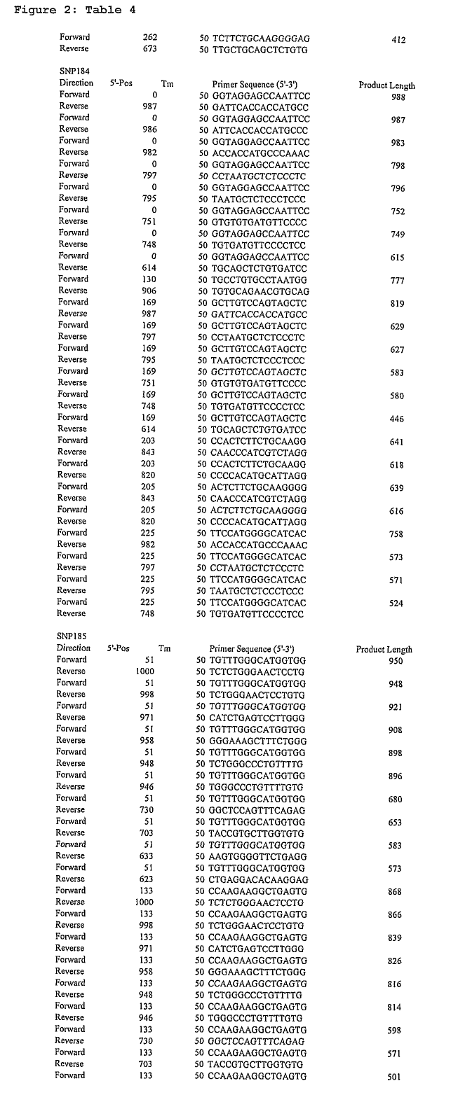

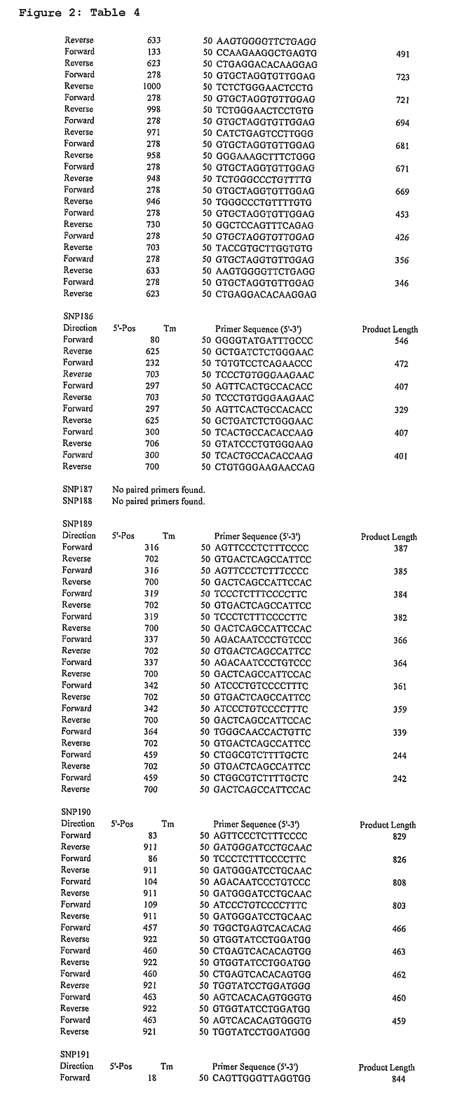

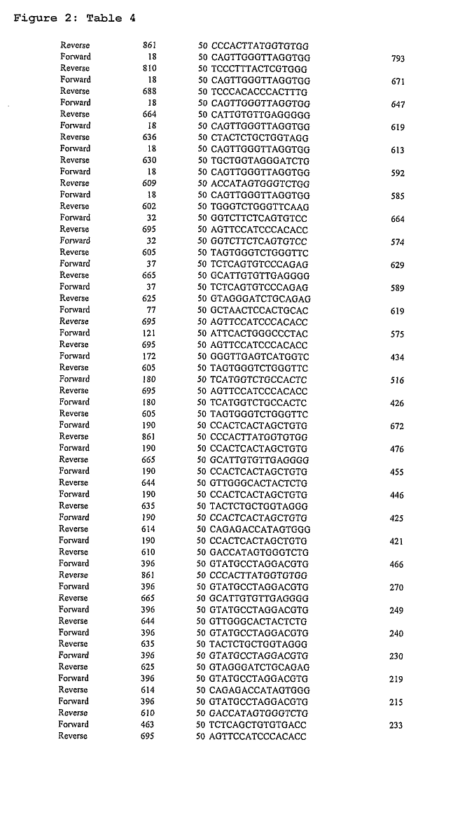

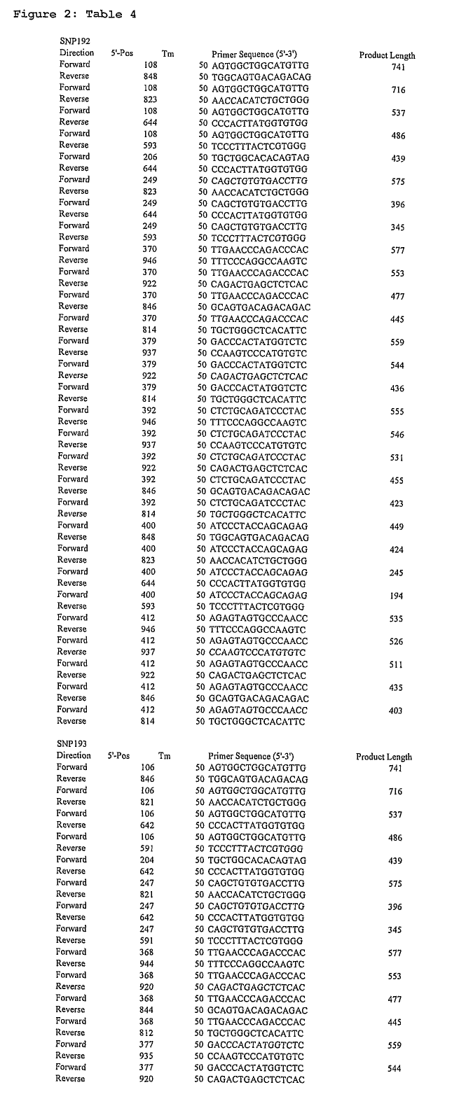

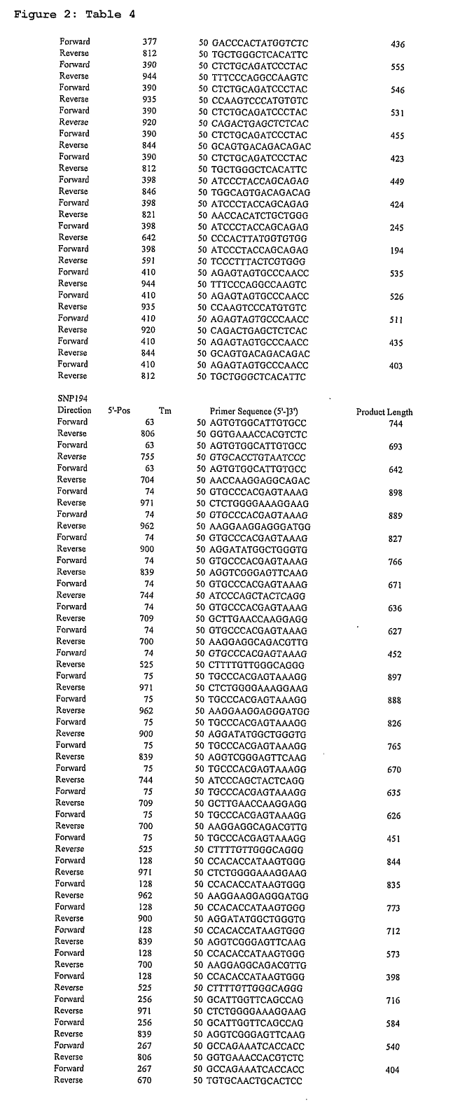

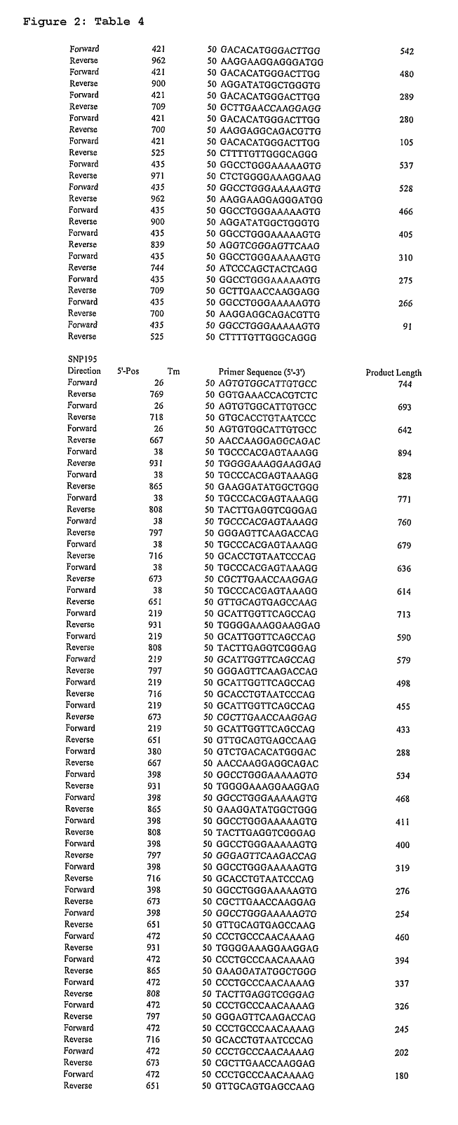

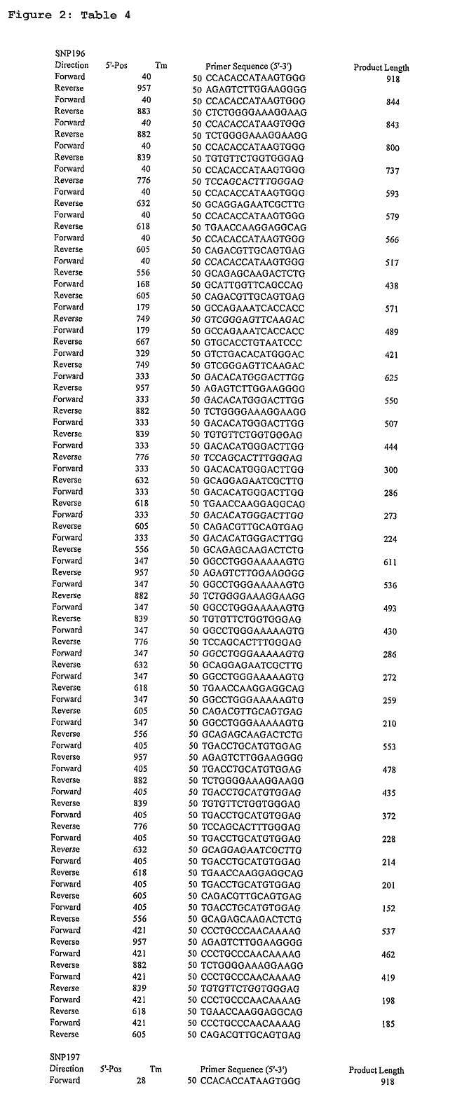

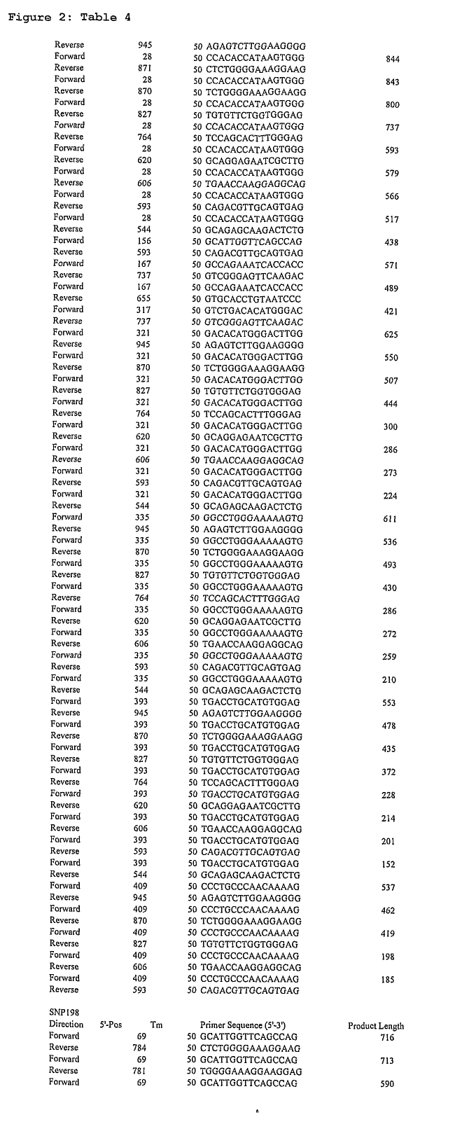

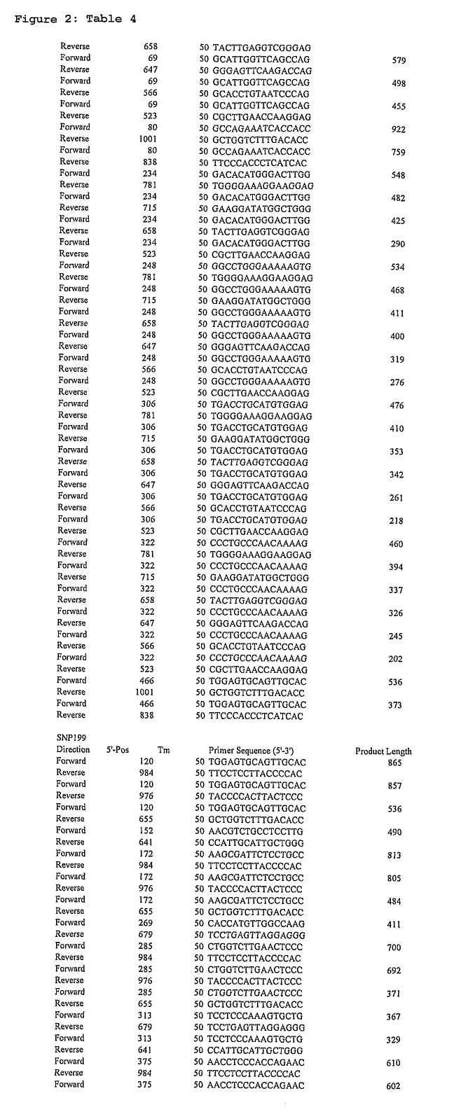

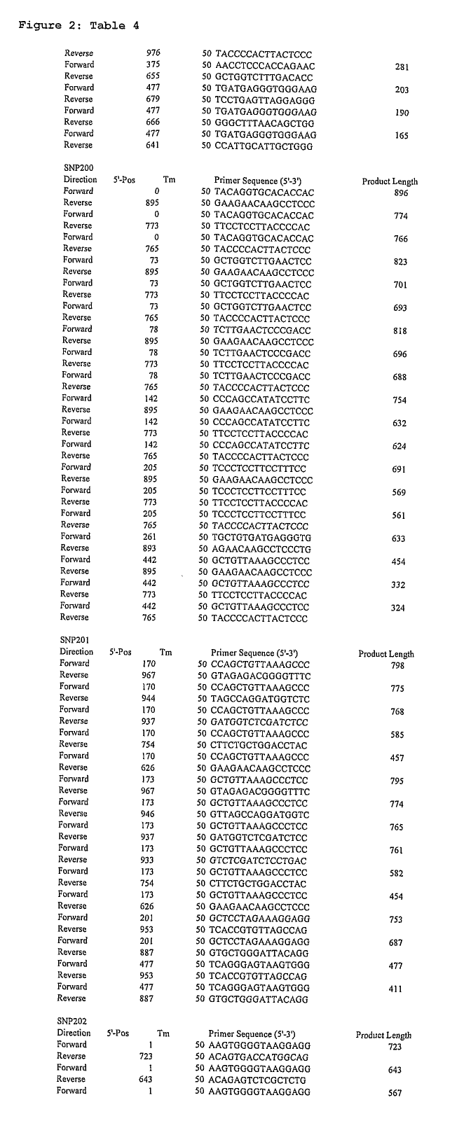

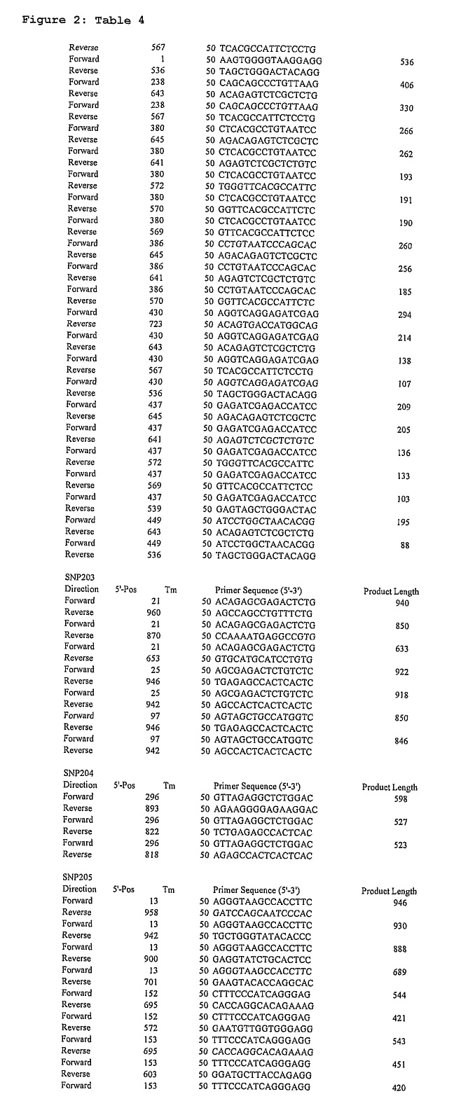

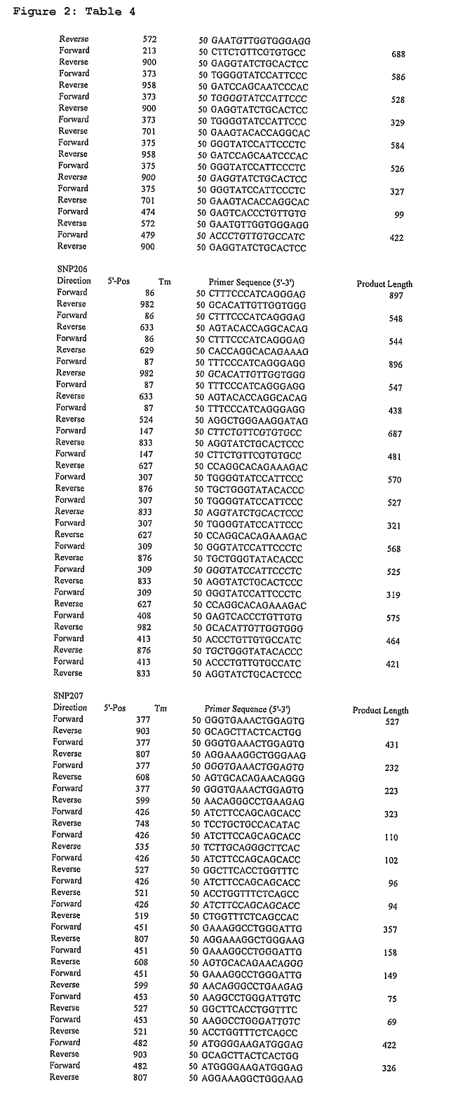

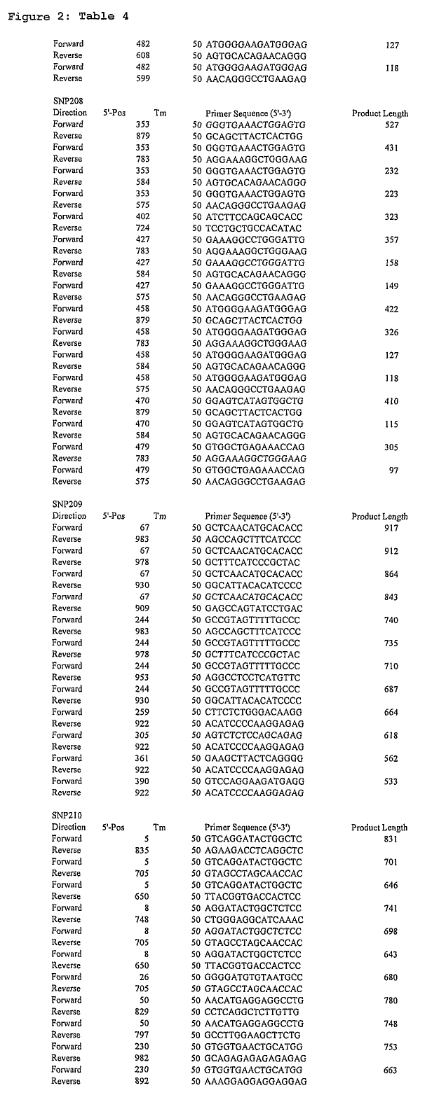

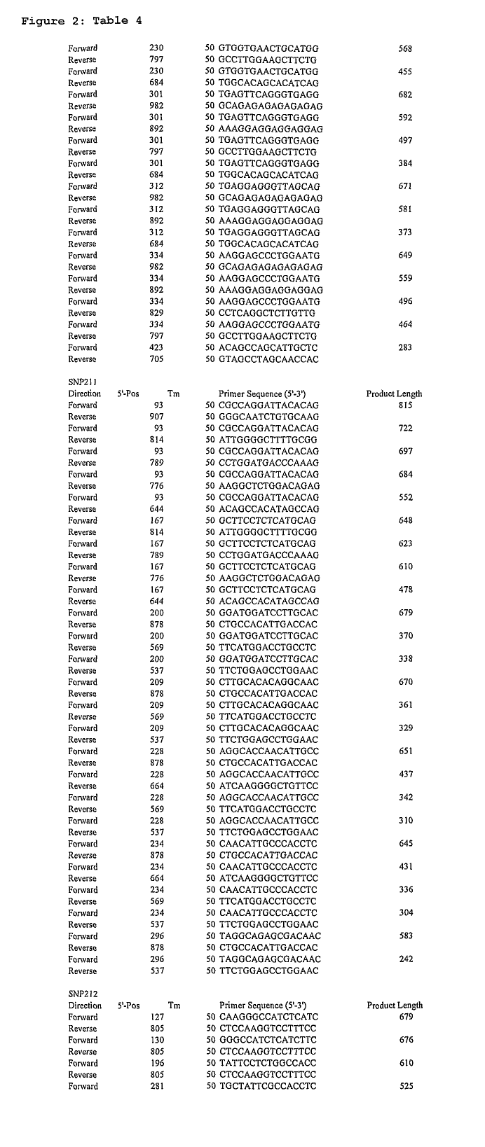

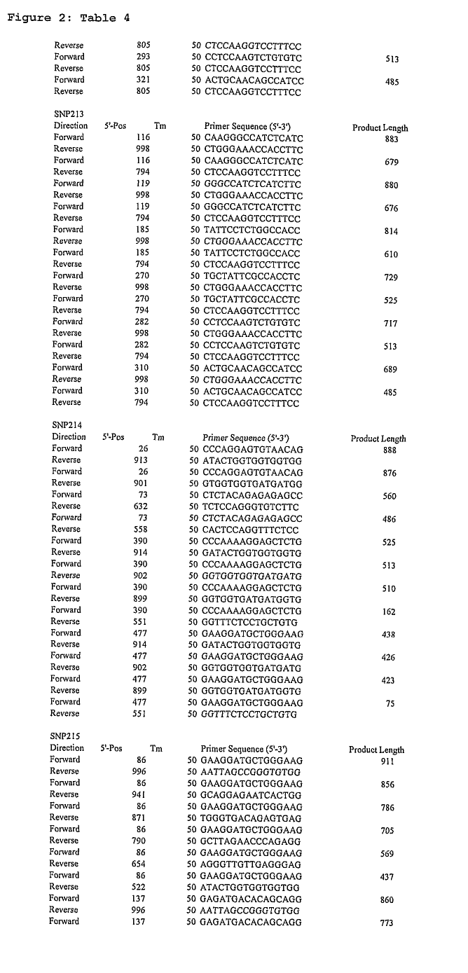

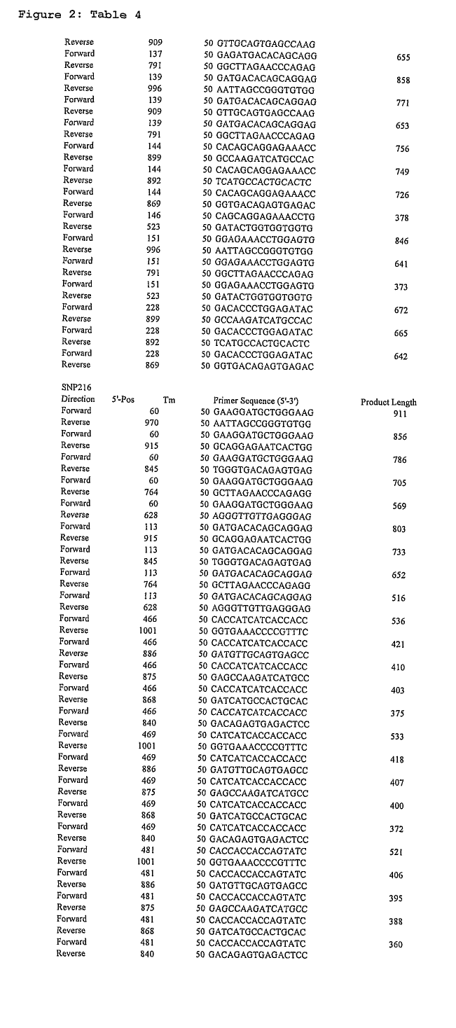

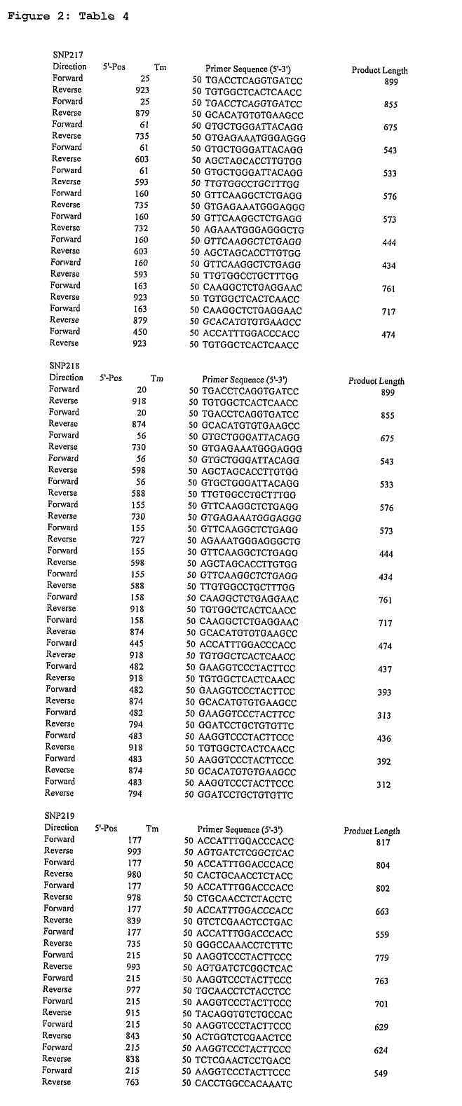

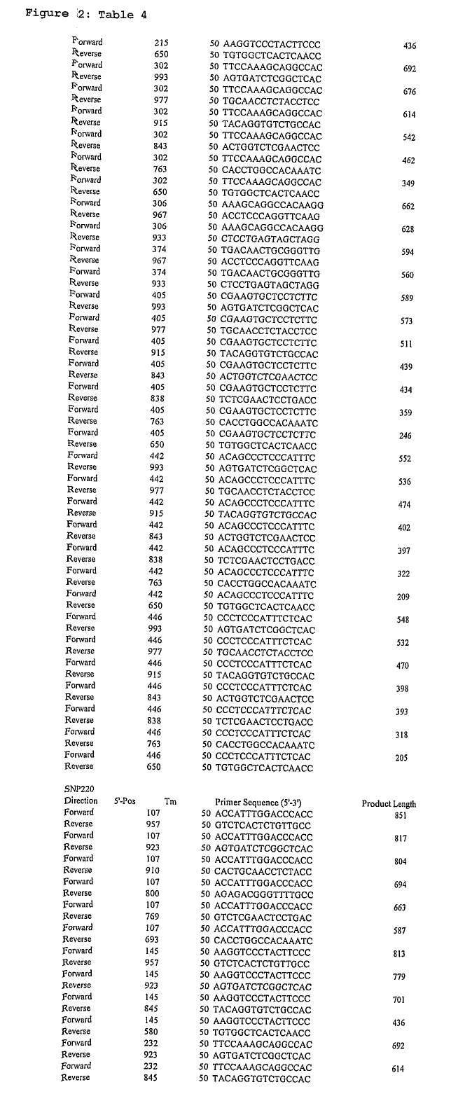

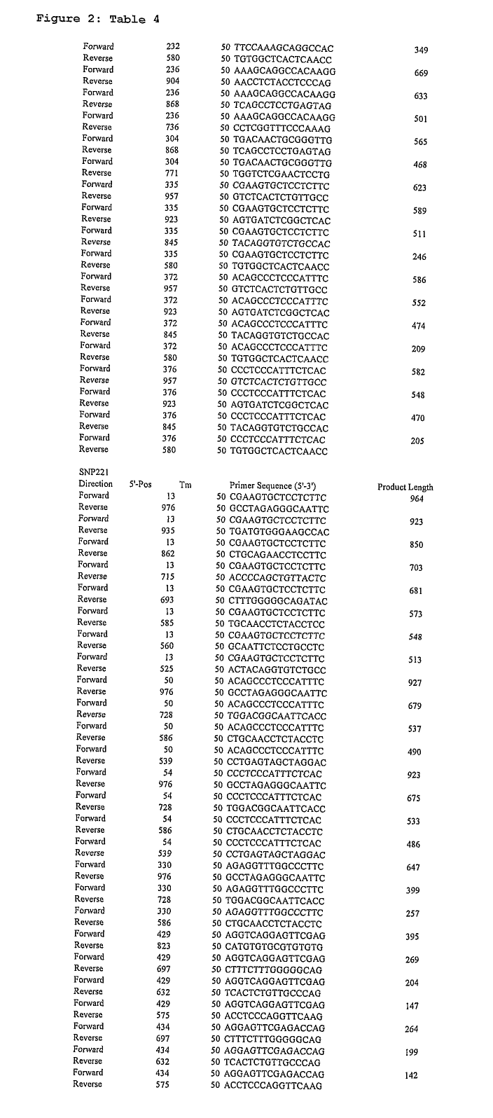

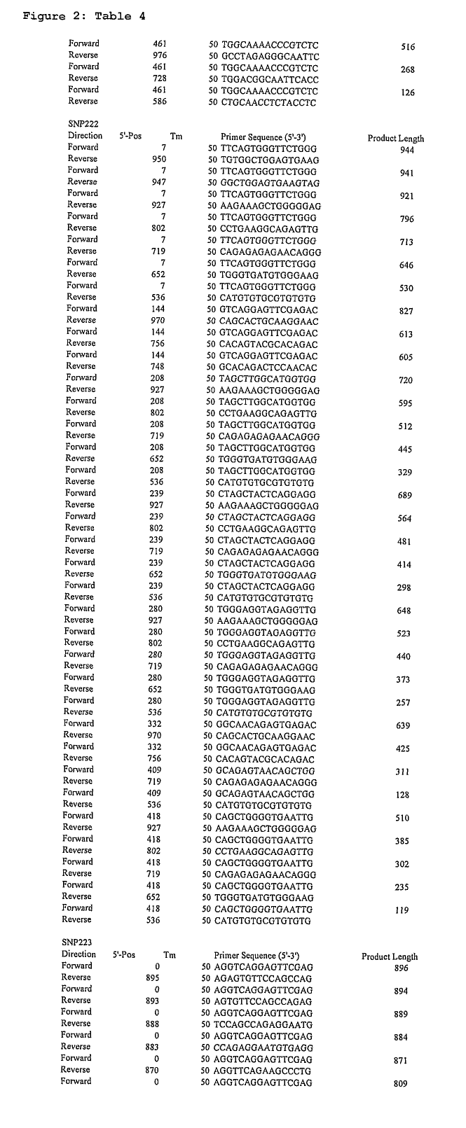

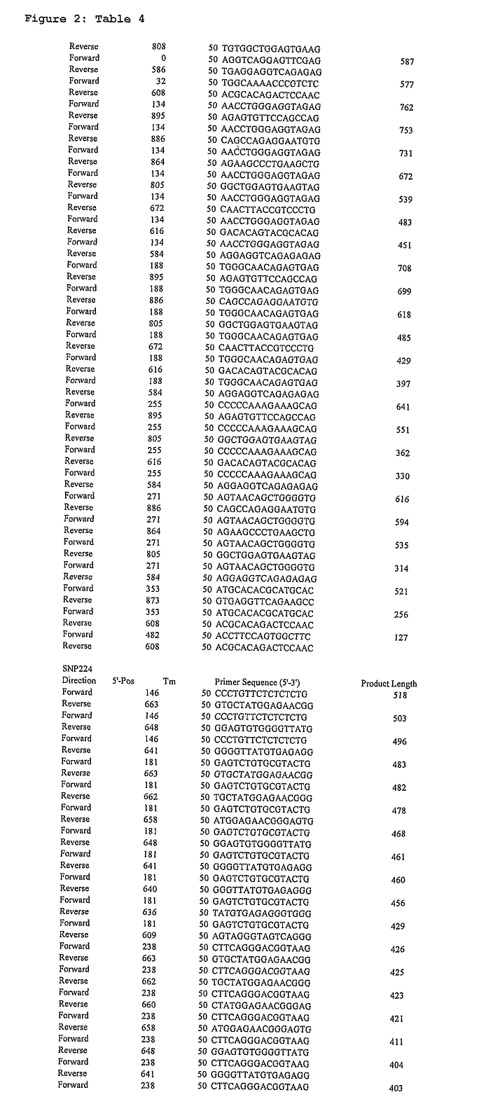

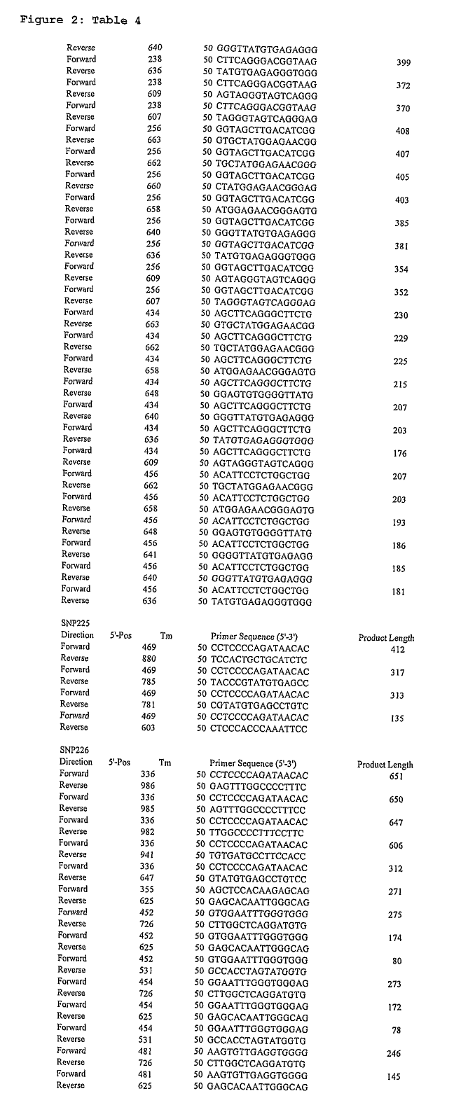

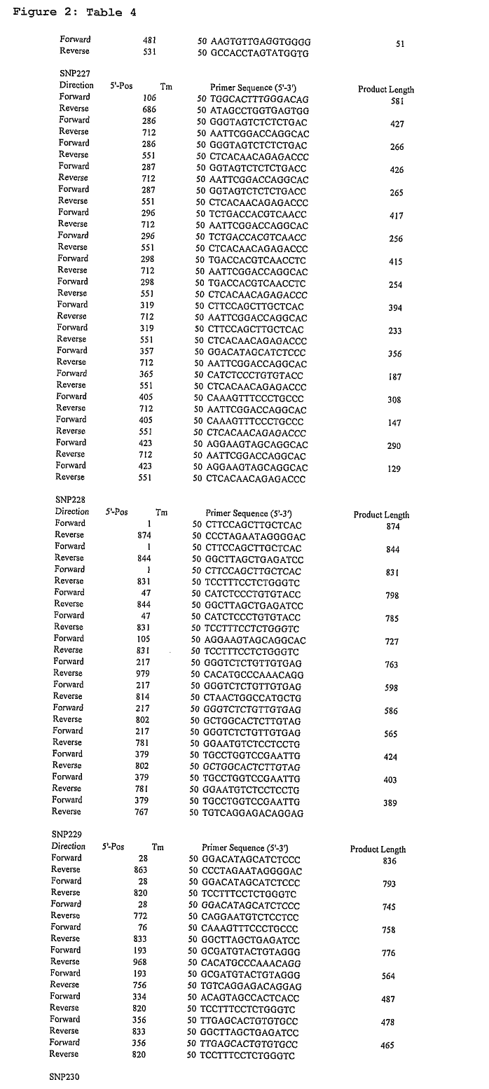

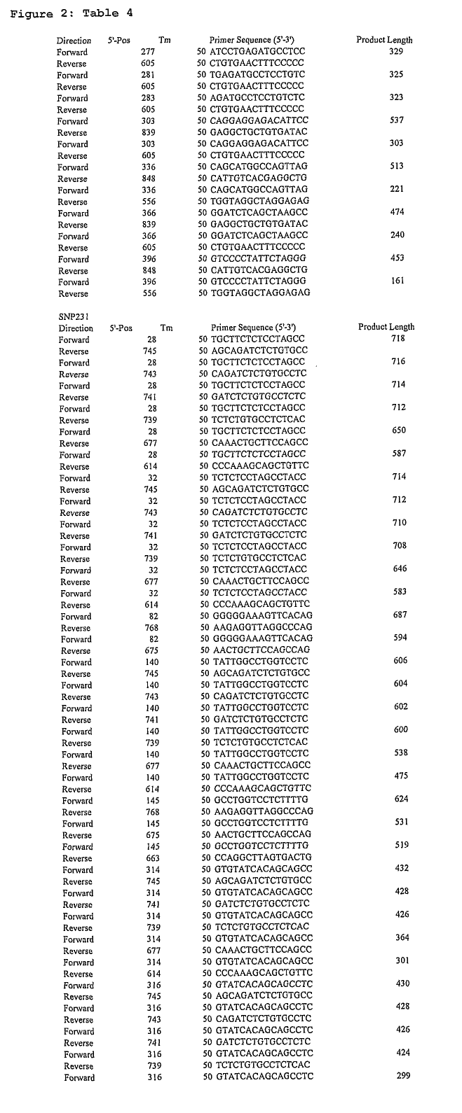

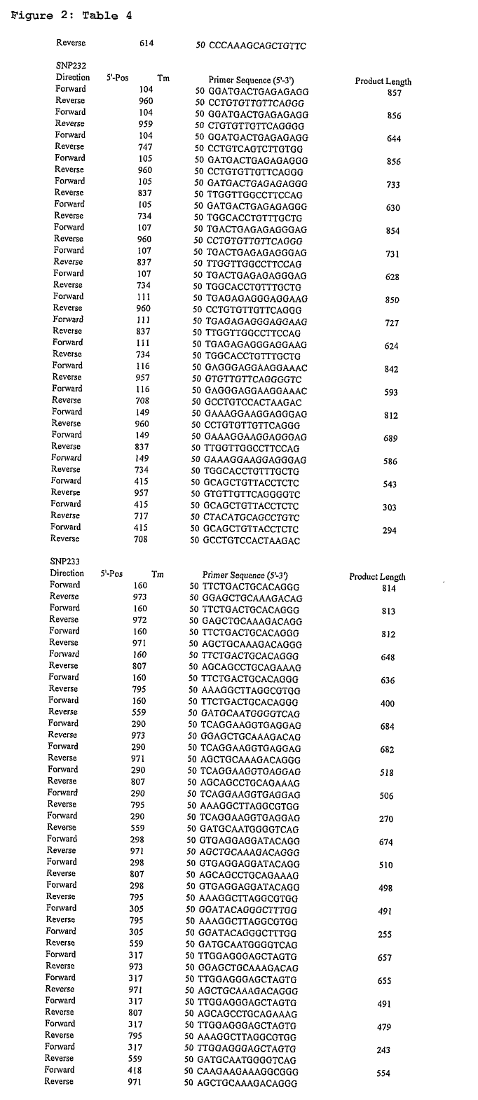

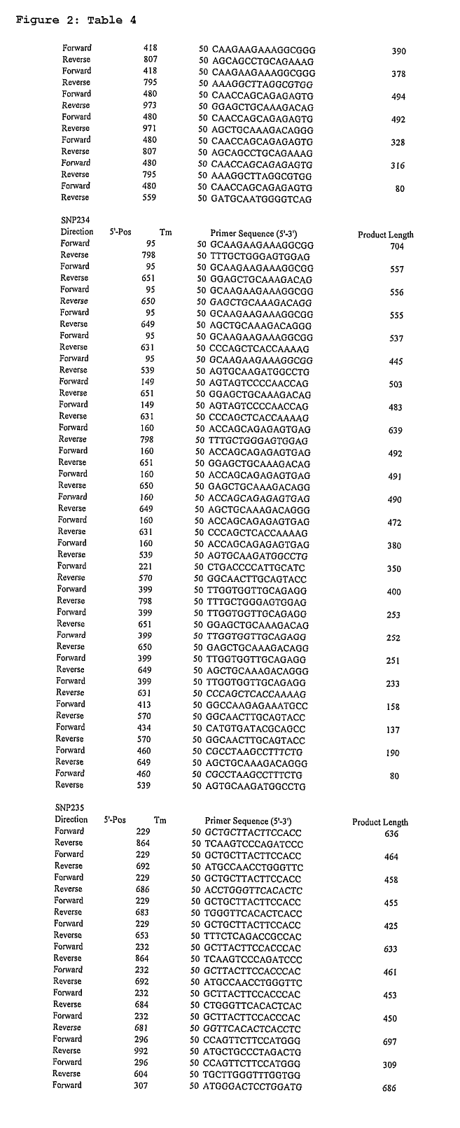

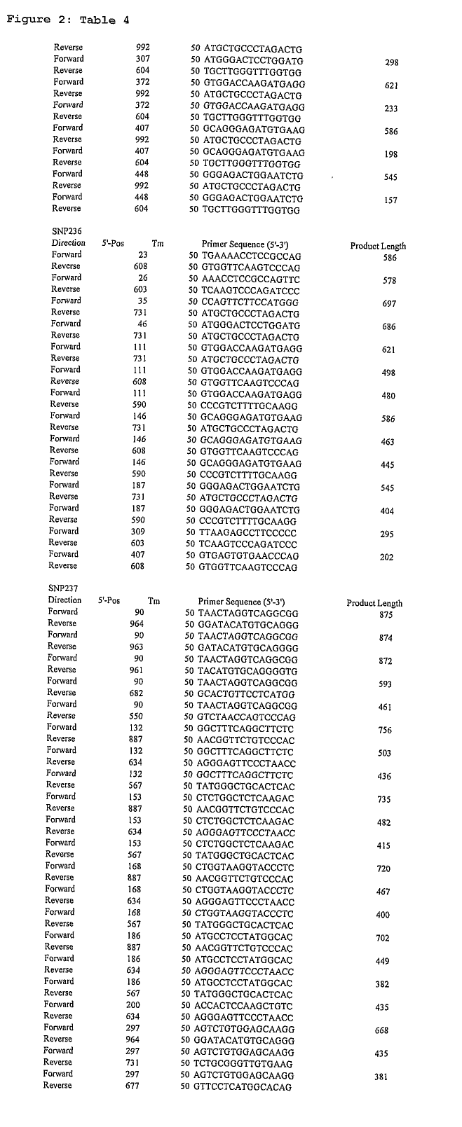

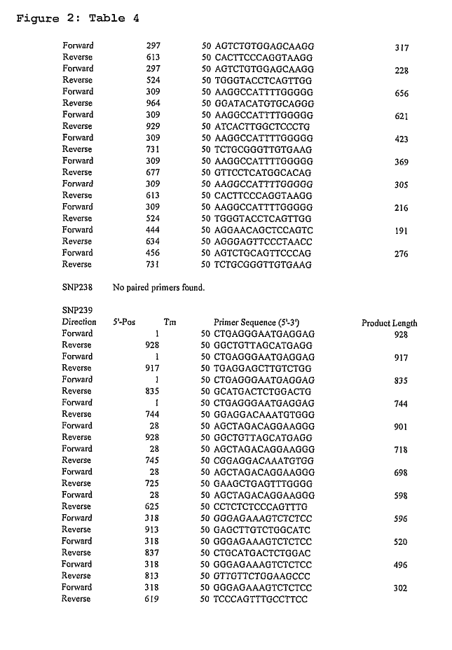

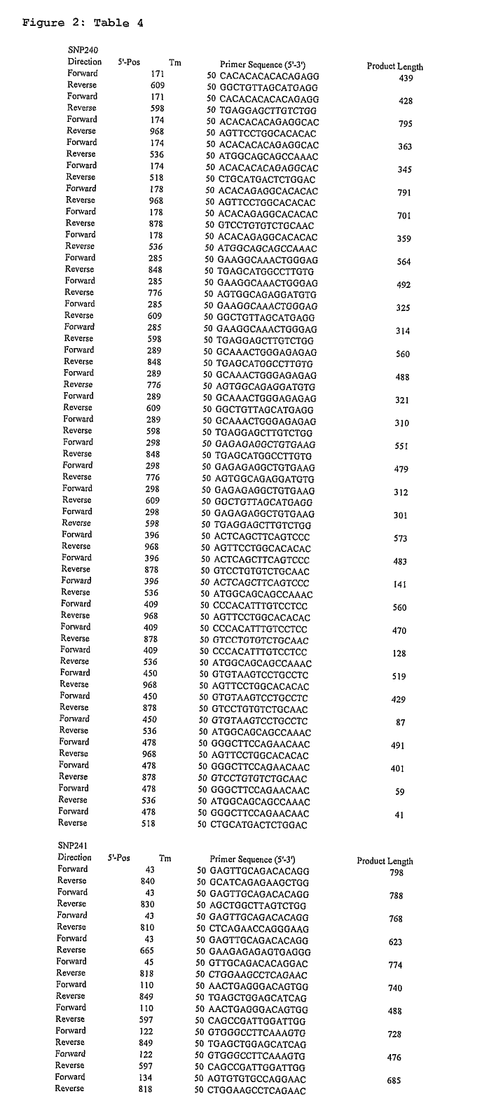

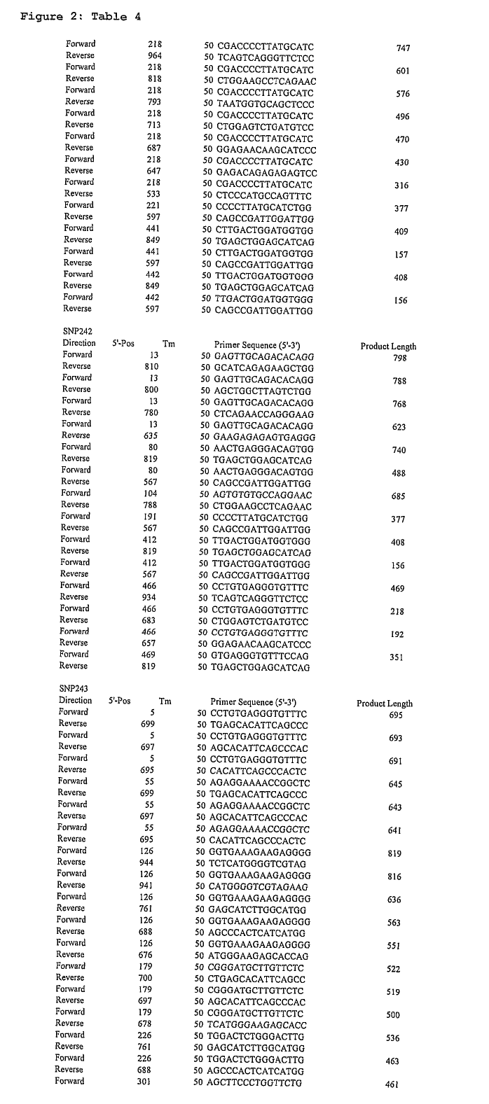

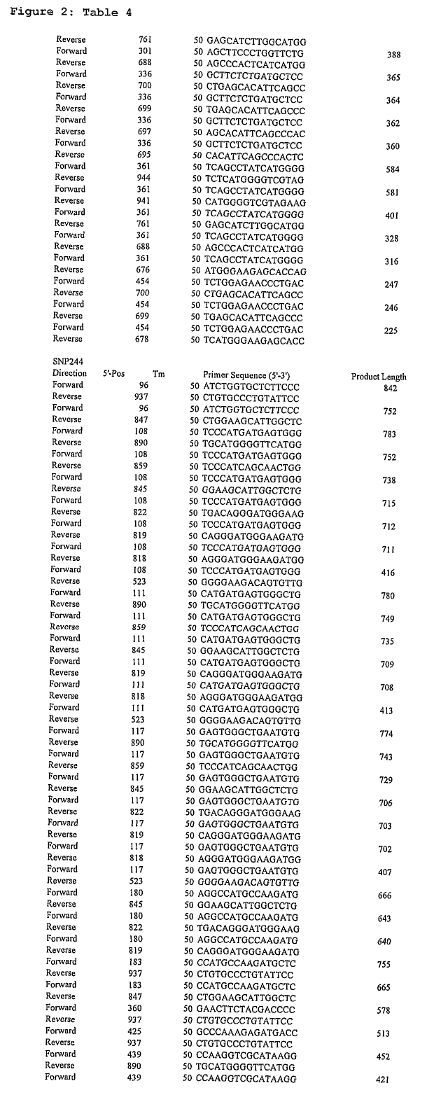

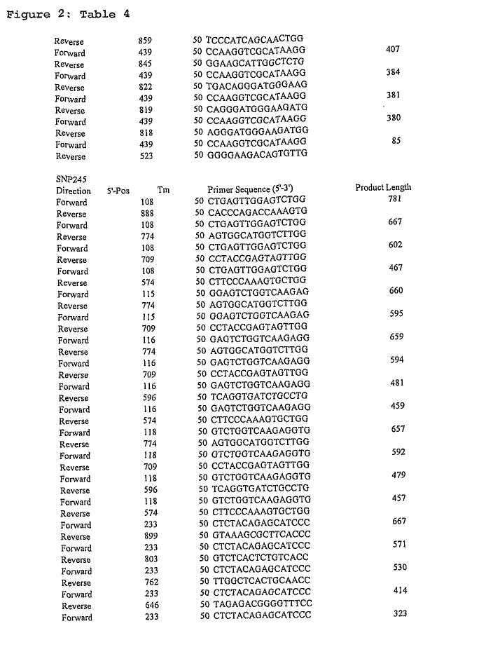

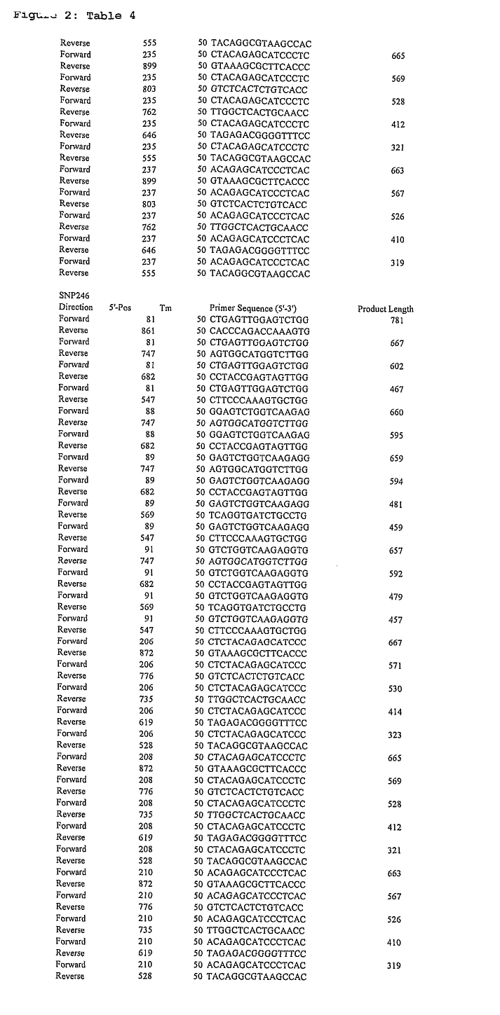

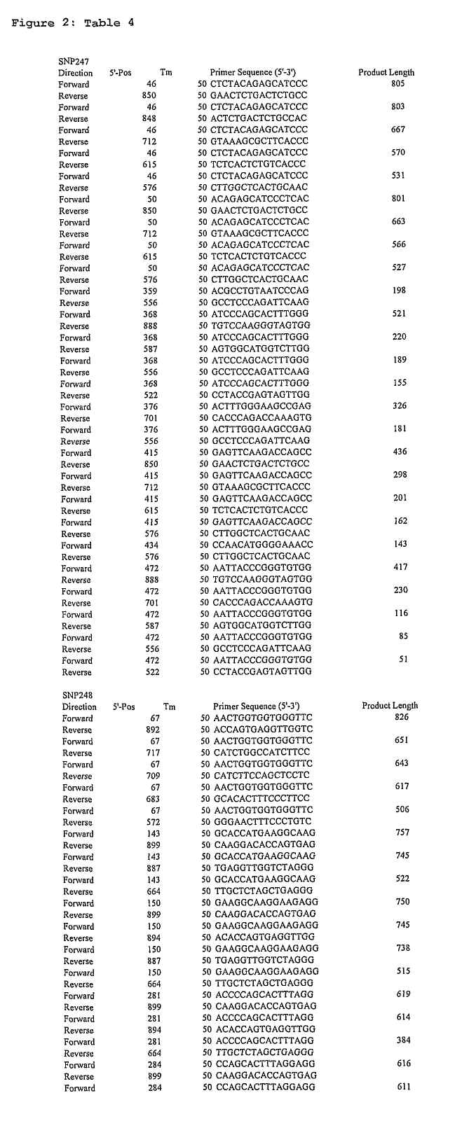

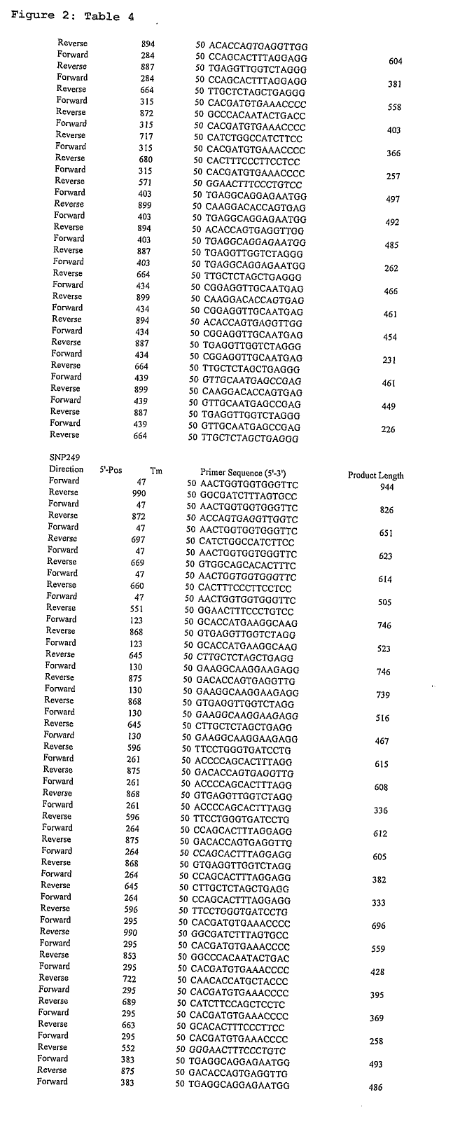

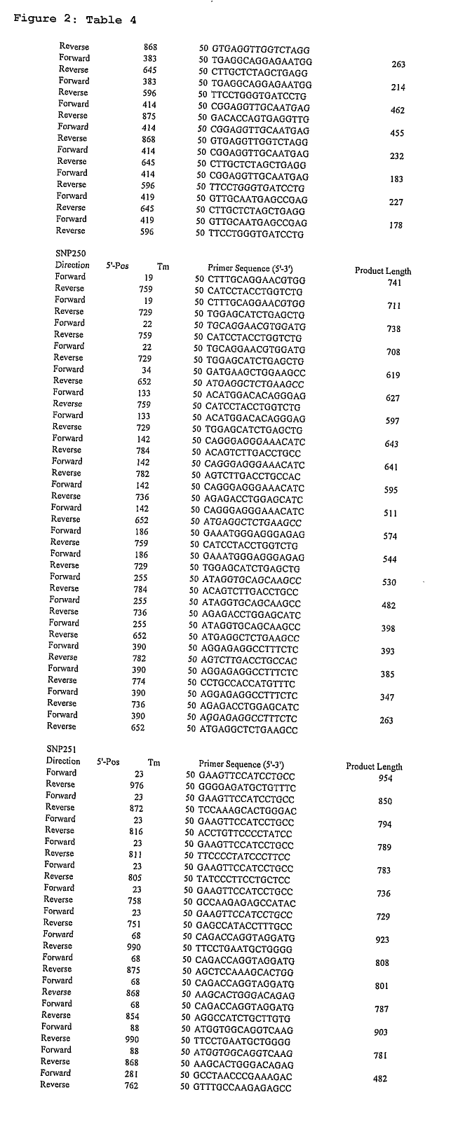

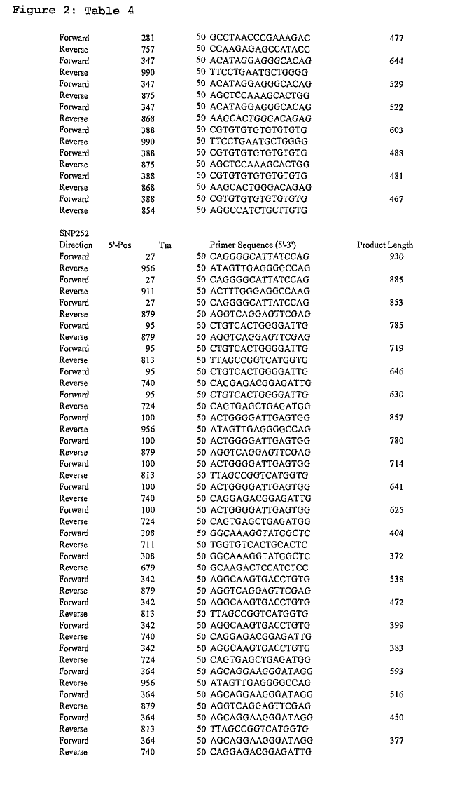

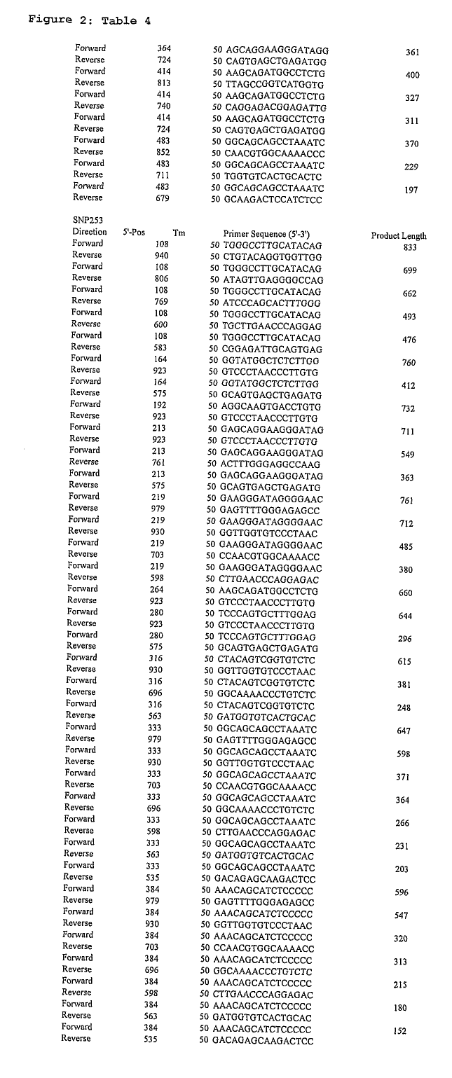

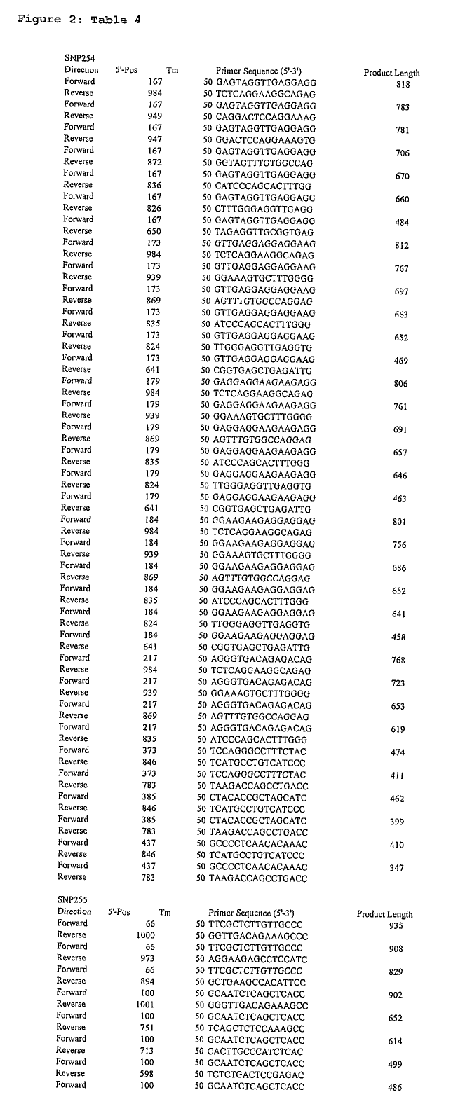

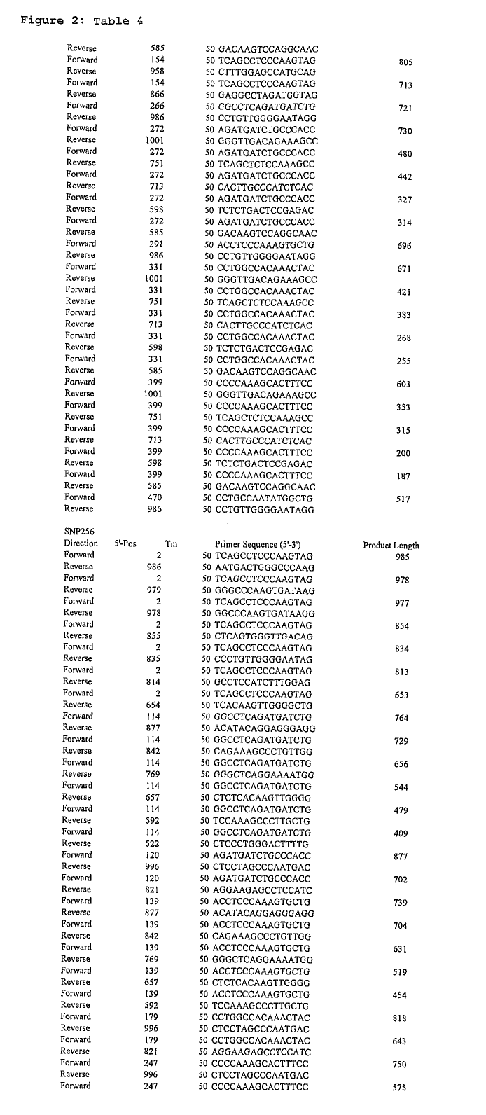

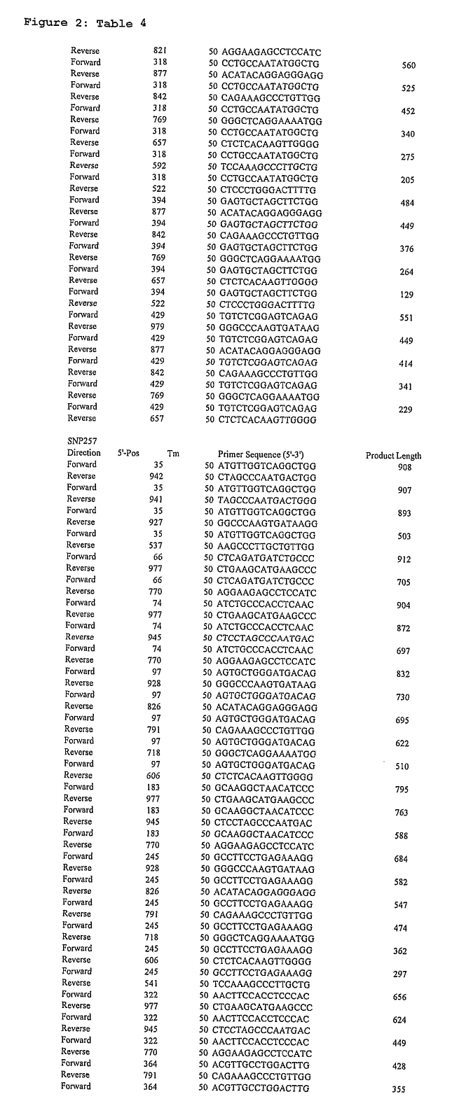

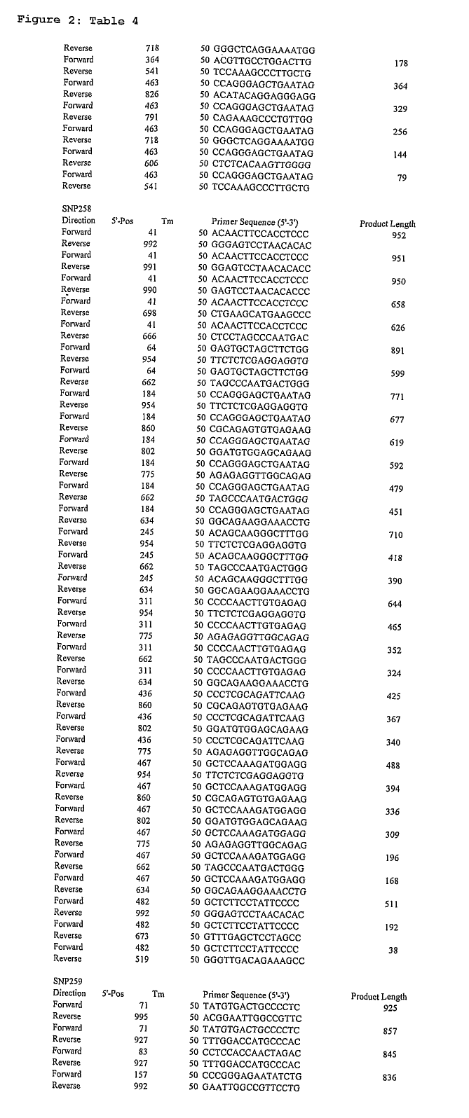

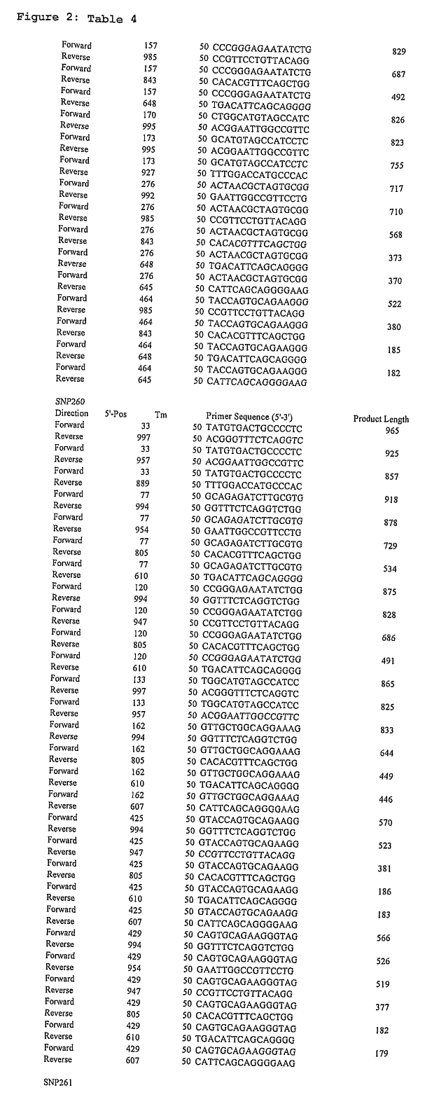

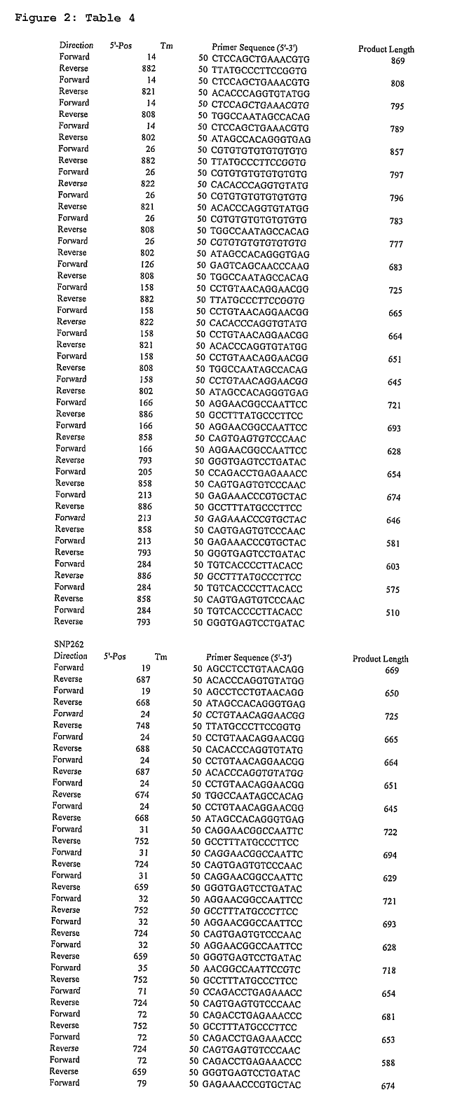

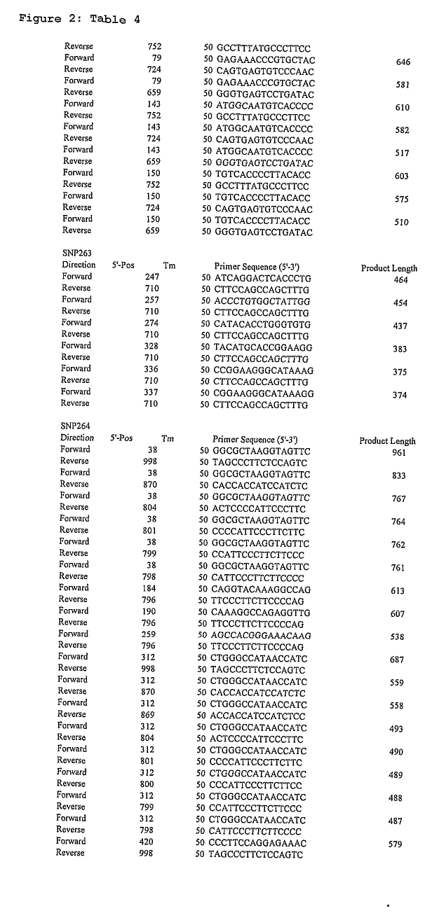

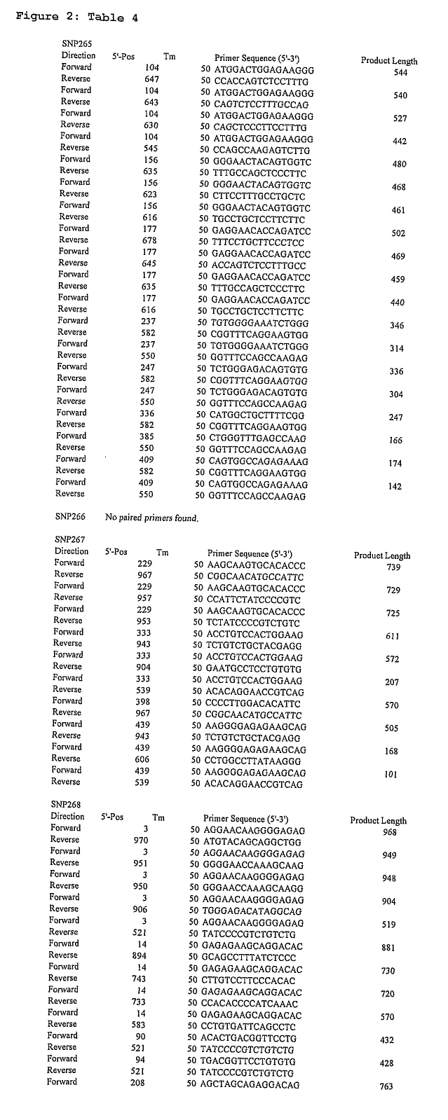

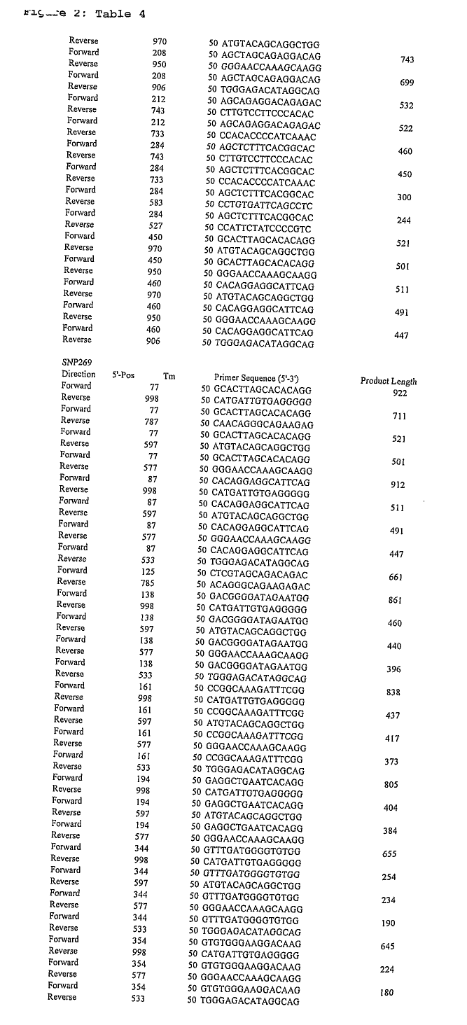

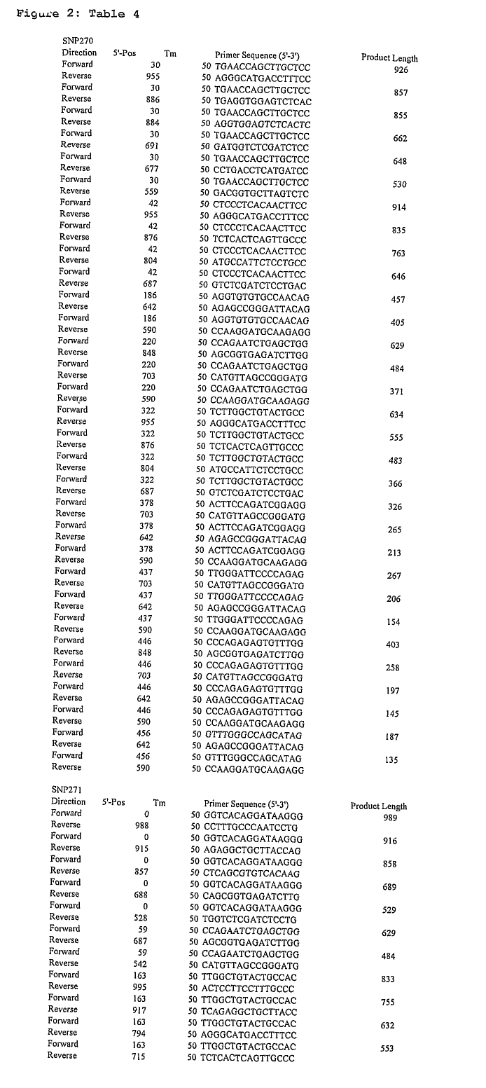

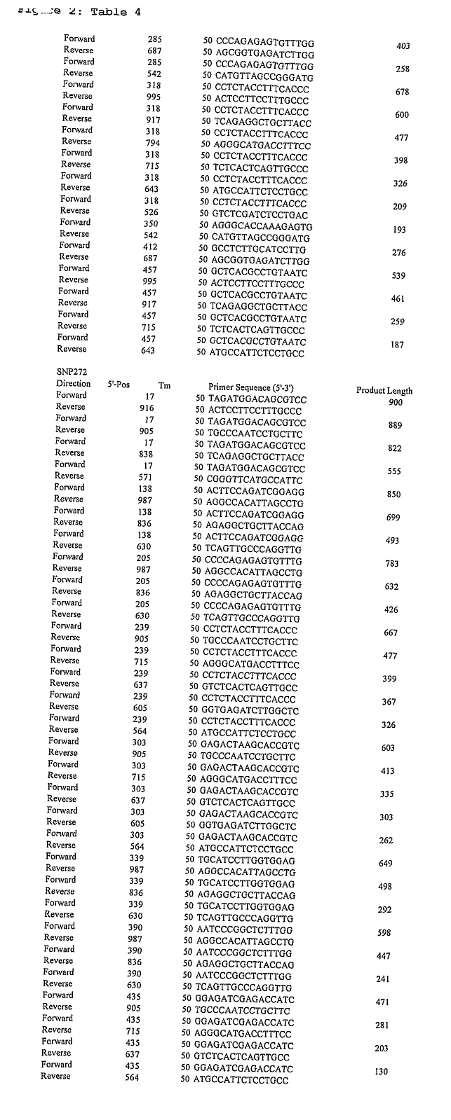

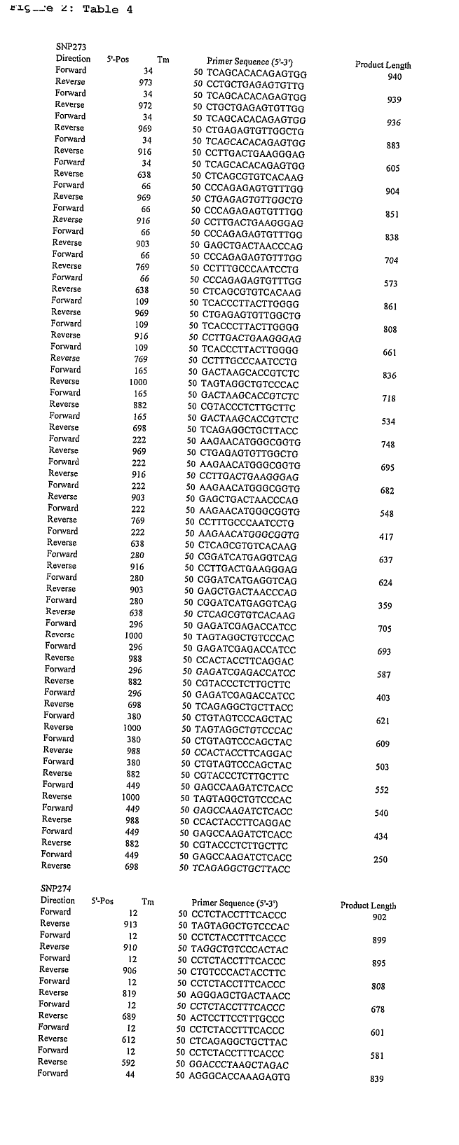

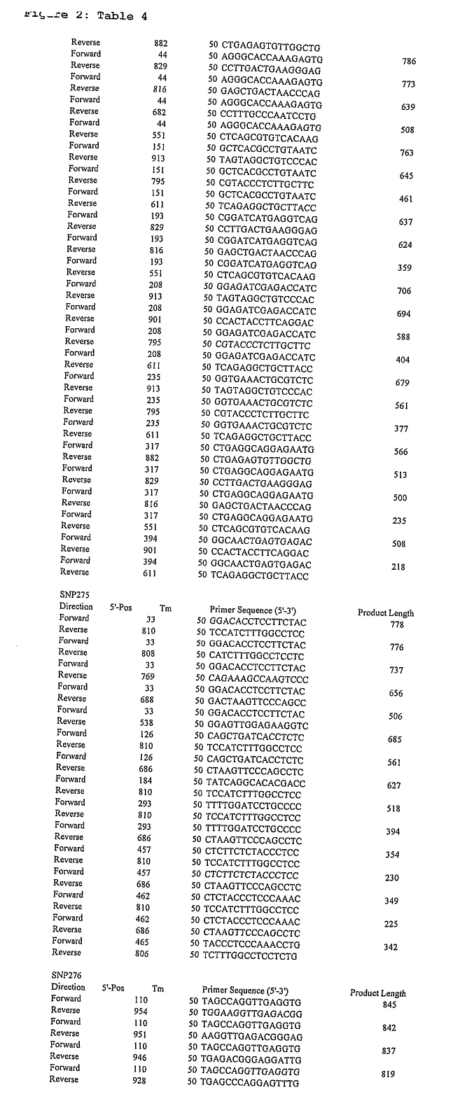

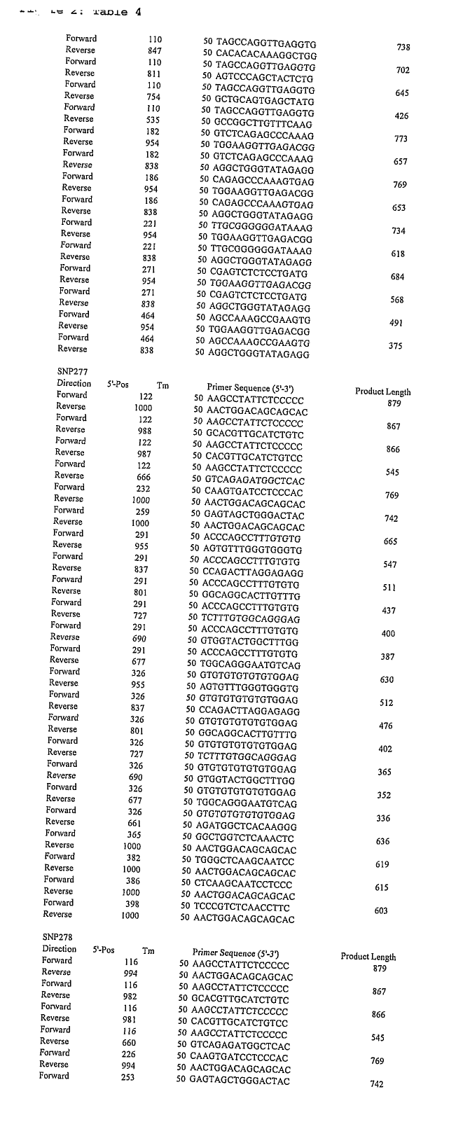

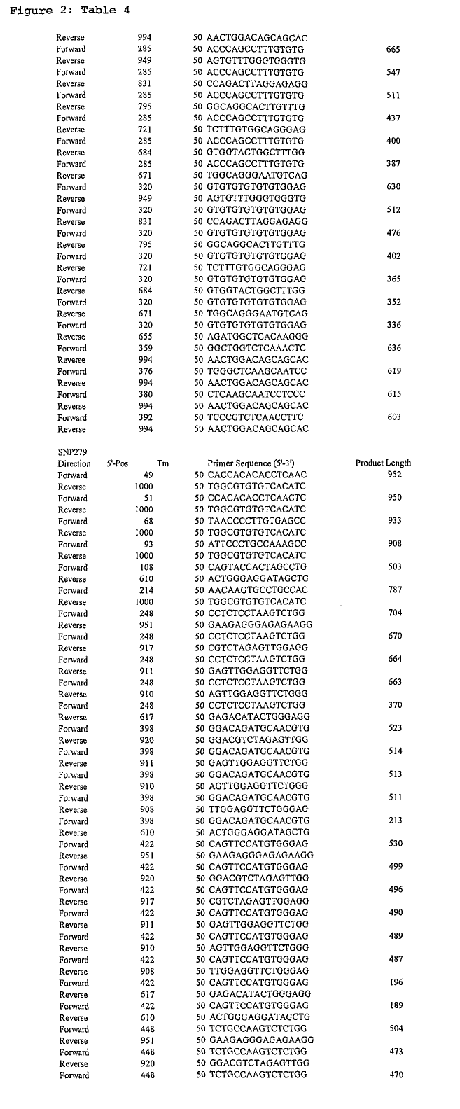

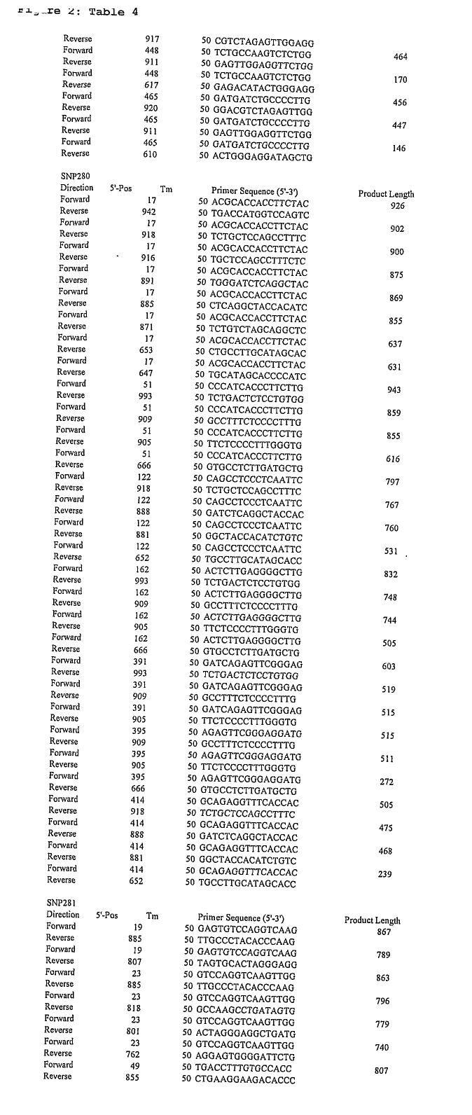

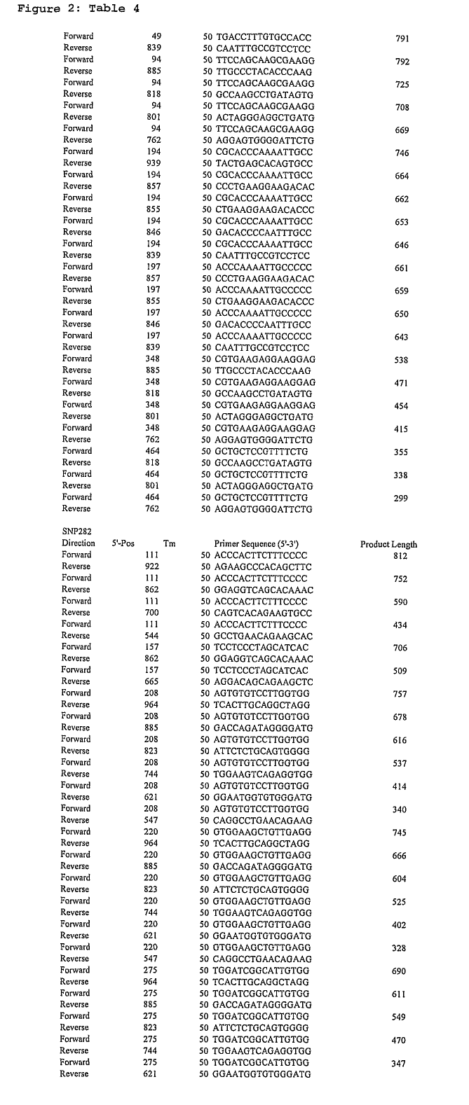

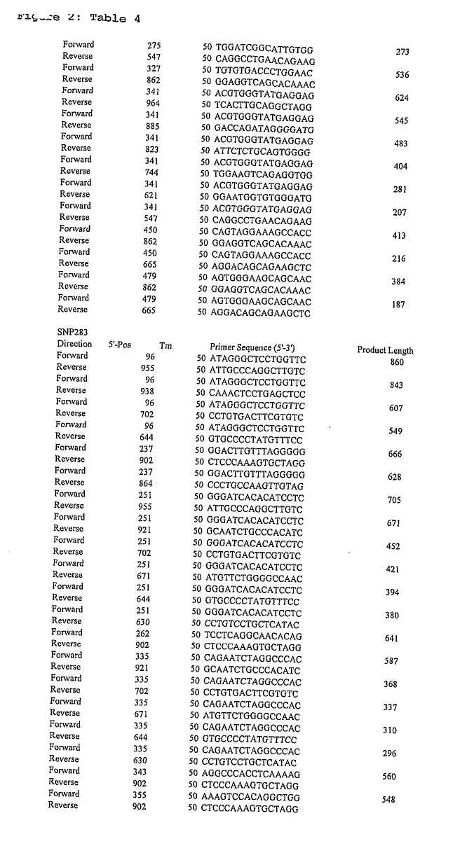

VNTR and SNP variances in this chromosomal location which may be used in the genetic tests of the invention were identified. VNTR motifs and surrounding sequences which may be used in the design of primers and/or probes are illustrated in Table 3 (FIG. 1). Primer and probe sets were designed around the SNPs to select the most useful for the diagnostic test (see Table 4 (FIG. 2)). The diagnostic test is designed to identify genotypes that correspond to or are predictive of the “low activity form” of the gene. The VNTRs and SNPs listed in Tables 3 and 4 (FIGS. 1 and 2) may affect gene expression or protein activity which alone or in combination could result in low activity of the MAO-B enzyme. The testing will also assess genetic marker haplotypes in the region which may have an influence on expression of the gene.

In accordance with the above, the present invention therefore also provides the use of a VNTR motif as listed in Table 3 (FIG. 1) herein in the assessment of MAO-B activity in an ADHD patient.

In particular, in this aspect, the present invention provides the use of a VNTR motif of Table 3 (FIG. 1) or a nucleic acid surrounding said motif in the generation of a probe and/or primer for the assessment of MAO-B activity, in particular the assessment of a genotype predictive of MAO-B activity, in an ADHD patient. The sequence surrounding the motif may be referred to as the flanking sequence and may extend from 1 to 250 bases upstream or downstream of the motif. Table 3 (FIG. 1) shows flanking sequences for the VNTR motifs in the Table.

Probes or primers generated using the VNTRs in Table 3 (FIG. 1) will be designed to measure the number of repeats that an individual has. The sequence around the VNTR, or flanking sequence (500 bp, see Table 3) may be used to design selective and sensitive primers/probes which can be used to detect the motif and determine the number of repeats.

A nucleic acid probe or primer generated using a VNTR motif in Table 3, may in one aspect comprise or consist (essentially) of:
(a) a fragment of the flanking sequence of the VNTR motif; or
(b) a nucleic acid sequence complementary to (a);
   wherein the VNTR motif is one listed in Table 3 and wherein the flanking sequence is the sequence from 1 to 250 bases upstream or downstream of the motif.

In these aspects of the invention, the preferred VNTR motifs are those with either or both of high repeat unit size and high copy number, such as for example: GCTGCCAAGAAGAAGGTG, TGGATGGATGAA, ACCATCATC, CACACACATG, ATTTATTAACT, TGTATCAGCCATTTCCAAC, TTTTACAAAGTAATATTTG, ATTTGTTTTACAAATTTTTACAAAGTA, ATAGATAT, TTCAAAGCAAATGTTGAG, TGTTTATGAAACAAA, GATTTCATTCATAAGATACAC and CTTGCTCAGTTACAAGA.

Typically a probe or primer has a length of from 15 to 60, such as from 20 to 50, 20 to 40, 15 to 30 or 15 to 40 bases. For example, a probe/primer may be 15, 16, 17, 18, 19, 20, 22, 25, 27 or 30 bases in length, typically 20 bases.

Furthermore the present invention provides the use of a primer or probe as identified in Table 4 (FIG. 2) herein in the assessment of MAO-B activity, in particular the assessment of a genotype predictive of MAO-B activity, in an ADHD patient.

As far as the sequences of the VNTRs and primers/probes are not previously known, the present invention also provides a VNTR or primer/probe having a sequence as listed in Tables 3 or 4 (FIGS. 1 and 2) respectively.

Preferably the VNTR motif is selected from those with either or both of high repeat unit size and high copy number, such as for example: GCTGCCAAGAAGAAGGTG, TGGATGGATGAA, ACCATCATC, CACACACATG, ATTTATTAACT, TGTATCAGCCATTTCCAAC, TTTTACAAAGTAATATTTG, ATTTGTTTTACAAATTTTTACAAAGTA, ATAGATAT, TTCAAAGCAAATGTTGAG, TGTTTATGAAACAAA, GATTTCATTCATAAGATACAC and CTTGCTCAGTTACAAGA.

The probes or primers of the invention, for example, those generated as described herein and those in Table 4, are useful in determining MAO-B activity in an ADHD patient. In particular, the probes/primers may be used to determine a genotype predictive of MAO-B activity. Thus the invention also provides a nucleic acid probe or primer of the invention for use in medicine. Also provided is the use of a probe or primer of the invention for the in vitro determination of a genotype predictive of MAO-B activity in a sample from an ADHD patient.

Further provided is a kit for use in diagnosing and/or treating ADHD in a subject which kit comprises a probe or primer of the invention. The kit may be used to assess MAO-B activity in subject such as an ADHD patient, for example by means of a genetic test carried out on a sample taken from the patient. Typically the kit is suitable for use in a method for determining whether an ADHD patient is suitable for treatment with an MAO-B inhibitor as described herein. Such a kit may additionally comprise, suitable nucleic acid labelling and/or detection means, reaction buffer, suitable enzymes and/or instructions for use.

It has also been shown that MAO-B activity is influenced by variants (genotypes) of the transcription factor AP-2beta (Damberg et al., 2000, Neurosci Lett. 291(3):204-6). Thus, the assessment of genotypes of this gene and of other factors affecting transcription of the MAO-B gene and MAO-B activity could be used to select ADHD patients for treatment with a MAO-B inhibitor. These tests may include assessment of genotypes or expression levels of transcription factors such as c-Jun, Egr-1 and Sp1 (see Wong et al., 2002, J. Biol. Chem. 277(25):22222-30). Also genes and proteins on the protein kinase C and MAPK signalling pathways were found to affect MAO-B gene expression (Wong et al., supra) and possibly activity, therefore also being of potential use in a patient selection test.

Genes or proteins involved in dopamine and PEA turnover may also influence MAO-B activity and therefore be of use in the selection of ADHD patients for treatment with MAO-B inhibitors. These include the dopamine transporter (DAT), Catechol-O-Methyltransferase and several other dopamine receptors (or the genes encoding these proteins).

Genetic tests can be carried out using DNA extracted from blood, including platelets, lymphocytes, saliva, urine, skin, hair or other body tissues. There are many methods which may be used to assess genotypes. The majority of these have been reviewed in the following publications: Mikkel et al., 2002, Psych. Genetics, 12(2):109-117; Dalma-Weiszhausz and Murphy, 2002, Psych. Genetics, 12(2):97-107 and Breen, 2002, 12(2):83-88. Examples of possible primers and primer combinations for genotyping tests to determine low MAO-B enzyme activity are detailed below in Table 4 (FIG. 2). VNTR and surrounding sequences upon which further primers and primer combinations may be designed are also detailed below in Table 3 (FIG. 1). Genetic tests can be carried out using DNA extracted from blood, including platelets, lymphocytes, saliva, urine, skin, hair or other body tissues. Similarly gene expression tests measuring the level of MAO-B messenger RNA expression may be measured in any human tissue samples as exemplified below.

MAO-B activity can also be measured indirectly through substrate measurement (or a combination of substrates), including PEA and/or HVA, (McKenna et al., Neurochem Res., (1993) 18(9):1023; Beckman et al. J Neural Transm., (1983) 57:103; Kennedy et al. Neurochem Res., (1993) 18(12):1281).

MAO-B inhibitors may represent an effective treatment for ADHD patients, and in particular in those patients demonstrating predominantly inattentive symptoms, with low MAO-B activity. The present inventors have suggested that reduced activity of the MAO-β isozyme will result in reduced levels of (HVA) and initially increased levels of extracellular and possibly intracellular dopamine. As individuals with low MAO-B enzyme activity have been exposed to this from an early age, it is not unlikely that a negative feedback system (possibly through dopamine presynaptic autoreceptors) will have been activated and that as a consequence dopamine turnover (synthesis and metabolism) will be reduced (Cooper et al. in The Biochemical Basis of Neuropharmacology, Oxford University Press, 5^{th}-8^{th} Edition, 1986-2002) in those patients with low MAO-B activity.

Accordingly, the method of diagnosis of the invention may be supplemented by a method of treating the patient with an MAO-B inhibitor.

Alternatively, the present invention provides the use of an MAO-B inhibitor in the manufacture of a medicament for the treatment of a patient suitable for treatment therewith, wherein the suitability of the patient for such treatment is determined by:
a) determining the level of MAO-B activity in an ADHD patient;
b) if said activity of MAO-B is within the 30% lower percentile of the full range of MAO-B activity within a normal population, testing said patient for symptoms of ADHD predominantly inattentive type (in accordance with DSM IV); and
c) if said activity of MAO-B falls within the 30% lower percentile of the full range of MAO-B activity within a normal population and said patient tests for symptoms of ADHD predominantly inattentive type, concluding that said patient is suitable for treatment with a monoamine oxidase type B inhibitor.

In its third aspect, the present invention provides the use of an MAO-B inhibitor in the manufacture of a medicament for the treatment of ADHD in a patient, wherein the MAO-B inhibitor is other than selegiline, pargiline or rasagiline.

MAO-B inhibitors suitable for use in these aspects of the invention are any compounds that have activity on the MAO-B enzyme. Preferably the MAO-B inhibitors suitable for use in these aspects of the invention are selective MAO-B inhibitors, i.e. these compounds will have affinity for the MAO-B enzyme but significantly less affinity for the MAO-A enzyme. Such MAO-B inhibitors typically include drugs such as selegiline, rasagiline, safinamide, mofegiline and lazabemide, amongst others, mainly developed and used to treat neurodegenerative disorders such as Parkinson's disease.

In the third aspect of the invention, a patient can have been diagnosed with any of the three sub-types of ADHD, that is either the ADHD predominantly inattentive type, the ADHD predominantly hyperactive-impulsive type, or the ADHD combined type. It is more preferred, however, that a patient will have been diagnosed with ADHD predominantly inattentive type. In a preferred embodiment of this aspect of the invention, a patient with ADHD will also have a low MAO-B activity. In this respect, a low MAO-B activity is as defined above with regard to the first aspect of this invention, i.e. enzyme activity levels within the 30% lower percentile of the full range of MAO-B activity within a normal population.

In accordance with this aspect of the invention, the MAO-B inhibitor may be any such inhibitor known in the art other than selegiline, pargiline or rasagiline. Typically such inhibitor may be SL-25.118, lazabemide, mofegiline, milacemide, LU-53439, SL-34.0026, EXP-631, M-2-PP, SL-25.1131, FA-87, RS-1636, NW-1048, himantane, excitatory amino acids, FA-73, ladostigil, CHF-3381, selegiline analogs, befloxatone, AIT-203 or AIT-297.

Preferably, the MAO-B inhibitor will be safinamide, mofegiline, lazabemide or a selegiline analog.

When the patient has been diagnosed with the ADHD predominantly inattentive type and demonstrates low MAO-B levels, the MAO-B inhibitor may alternatively be, amongst others, selegiline.

Selegiline

The irreversible MAO-B inhibitor selegiline (L-deprenyl) may be used to treat ADHD patients with low MAO-B activity and the predominantly inattentive subtype of ADHD.

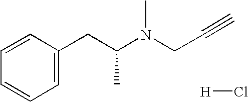

The molecular structure of selegiline.

There have been some studies which have evaluated the use of selegiline in ADHD (Wood et al. Psychopharmacol. Bull., 1983, 19:627; Wender et al. Psychopharmacol. Bull., 1985, 21:222; Rapoport et al. Psychopharmacol. Bull., 1985, 21:232; Ernst et al. Psychopharmacol. Bull., 1996, 32:327; Akhondzadeh et al., Prog. Neuropsycholpharmacol Biol. Psychiatry, 2003, 27(5):841-5), however the majority of these demonstrated that selegiline was of less benefit to the patients than psycho-stimulant treatment.

Selegiline exists in a number of different formulations as Eldepryl (coated tablet formulation), Zelapar (fast-dissolving Zydis formulation, WO-09626720), Emsam (patch formulation, WO-09426218), Selegiline XR (extended release formulation, U.S. Pat. No. 5,484,608) any of which may be used according to the various embodiments of the invention.

A dose varying between 2-20 mg/dag (Eldepryl or bioequivalent doses of the other formulations) may be used for the treatment.

Rasagiline

Rasagiline (EP-00436492) is a selective and irreversible MAO-B inhibitor which may be used to treat a subgroup of ADHD patients with low MAO-B activity and the predominantly inattentive subtype of ADHD.

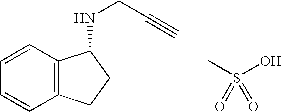

The molecular structure of rasagiline.

A dose of 1-2 mg/day may be used for the treatment.

Other MAO-B Inhibitors

Further compounds that inhibit MAO-B activity and that may, therefore be used in the embodiments of the present invention are illustrated in Table 2.

TABLE 2

| Drug | Patent | Company |
|---|---|---|
| SL-25.1188 | | Sanofi-Synthelabo |
| lazabemide | DE-03530046 | Roche Holding AG |
| mofeglline | | Hoechst Marion Roussel Inc |
| milacemide | DE-03010599 | GD Searie & Co |
| LU-53439 | | Knoll Ltd |
| SL-34.0026 | | Synthelabo |
| EXP-631 | | Bristol-Myers Squlbb Pharma Co |
| M-2-PP | | DRAXIS Health Inc |
| MAO-B inhibitors, Bari University | | Universita di Bari |
| SL-25.1131 | | Sanofi-Synthelabo |
| FA-87 | | Universitat Autonoma de Barcelona |
| selegiline analogs, Draxis | | DRAXIS Health Inc |
| RS-1636 | | Sankyo Co Ltd |
| NW-1048 | | Newron Pharmaceuticals SpA |
| himantane | | Russian Academy Medical Science |
| excitatory amino acids, NPS Allelix/Ell Lilly | | NPS Allelix Corp |
| FA-73 | | Universitat Autonoma de Barcelona |
| ladostigil | | Hebrew University of Jerusalem |
| CHF-3381 | | Chiesi Farmaceuticl SpA |
| safinamide | | Pharmacia & UpJohn AB |
| selegiline transdermal system, Somerset | WO-09426218 | Somerset Pharmaceuticals Inc |
| — | WO-2004026825 | F Hoffmann-La Roche Ltd |
| — | WO-2004026826 | F Hoffmann-La Roche Ltd |
| — | WO-2004026827 | F Hoffmann-La Roche Ltd |
| — | WO-2004014856 | F Hoffmann-La Roche Ltd |
| — | WO-2004007429 | F Hoffmann-La Roche Ltd |
| — | WO-03099763 | F Hoffmann-La Roche Ltd |
| — | WO-03091219 | F Hoffmann-La Roche Ltd |
| — | WO-03075906 | Somerset Pharmaceuticals Inc |
| — | WO-03072055 | Teva Pharmaceuticals Inc |
| — | WO-03066596 | F Hoffmann-La Roche Ltd |
| — | WO-03039525 | Krele Pharmaceuticals |
| — | WO-02083656 | Societe de Conseils de Recherches et |
| | | d'Applications Scientifique |
| — | WO-02068376 | Finetech Laboratories Ltd |
| — | WO-00219964 | Somerset Pharmaceuticals Inc |
| — | WO-00215841 | Pelyipharm SA |
| — | US-06350876 | Kuraray Co Ltd |
| — | WO-00136407 | Societe de Conseils de Recherches et |
| — | WO-00134172 | d'Applications Scientifique |
| | | Vela Pharmaceuticals Inc |
| — | WO-00126656 | Societe de Conseils de Recherches et |
| | | d'Applications Scientifique |
| befloxatone | WO-00112176 | Sanofi-Synthelabo |
| — | WO-00107033 | Chinoin Gyogyszer Es Vegyeszeti |
| — | WO-00071109 | Somerset Pharmaceuticals Inc |
| AIT-203 | WO-09957119 | Spectrum Pharmaceuticals Inc |
| AIT-297 | | |
| — | WO-09936513 | AuRx Inc |
| — | WO-09903458 | R P Scherer Ltd |
| — | US-05840979 | University of Saskatchewan |
| — | EP-00878191 | IIP Institut Fuer Industrielle Pharmazie Forschungs- |
| | | Und Entwicklungsgesellschaft mbH |
| — | WO-09822110 | Virginia Tech Intellectual Properties Inc |
| — | WO-09733572 | Somerset Pharmaceuticals Inc |
| — | US-05668154 | Hoechst Marion Roussel Inc |
| — | WO-09717346 | Synthelabo |
| — | WO-09637199 | Technion Research & Development |
| | | Foundation Ltd; Teva Pharmaceutical |
| | | Industries Ltd |
| — | WO-09635425 | Individual |
| — | WO-09626720 | R P Scherer Ltd |
| — | WO-09624349 | Consejo Superior De Investigaclones |
| | | Cientificas; Universitat Autonoma de |
| | | Barcelona |
| — | WO-09619981 | Orion Corp |
| — | WO-09612472 | Chinoin RT |
| — | EP-00699680 | Synthelabo |
| — | EP-00670313 | Takeda Chemical Industries Ltd |
| — | WO-09519960 | Laboratoires Mayoly Spindler SARL |
| — | EP-00655445 | Synthelabo |
| — | WO-09511016 | Technion Research & Development |
| | | Foundation Ltd; Teva Pharmaceutical |
| | | Industries Ltd |
| — | WO-09508325 | Merrell Dow Pharmaceuticals Inc |
| — | US-05380755 | Bristol-Myers Squibb Pharma Co |
| — | WO-09324120 | Merrell Dow Pharmaceuticals Inc |
| — | WO-09312775 | Chinoin Gyogyszer Es Vegyeszetl |
| — | EP-00538134 | Teva Pharmaceutical Industries Ltd |
| excitatory amino acids, NPS Allelix/Eli Lilly | EP-00529994 | NPS Allelix Corp |
| excitatory amino acids, NPS Allelix/Eli Lilly | EP-00529995 | NPS Allelix Corp |
| — | WO-09221333 | Shire Laboratories Inc |
| — | EP-00504574 | Wakamoto Pharmaceutical Co Ltd |
| — | WO-09215551 | University of Saskatchewan |
| — | WO-09108201 | Delalande SA |
| — | US-04971995 | Delalande SA |
| — | EP-00385210 | Hoffmann-La Roche AG |
| — | EP-00382533 | Consejo Superior De Investigaclones Cientificas |

In accordance with the present invention, an MAO-B inhibitor, such as selegiline or rasagiline, may be in any appropriate formulation. For example, an inhibitor may be formulated for oral inhalation, intranasal, intravenous, buccal, lingual, sublingual, dermal or intramuscular administration. Oral formulations include liquids or gel capsules. Dermal formulations include patch formulations.

One example of suitable inhalation drug delivery technology is the Staccato™ drug delivery device. The Staccato™ technology comprises a hand-held system providing rapid, reliable deep lung delivery of a drug using a thermally-generated condensation aerosol. A single inhalation actuates the controlled, rapid heating of a thin layer of pure (additive-free) drug on a metal substrate. The heat vaporises the drug into the device airstream where the resulting gas-phase molecules condense into appropriate size aerosol particles for deep lung delivery and absorption into systemic circulation. The time from the breath-activated substrate heating to drug entry into respiratory tract is less than 1 second.

One example of suitable intranasal drug delivery technology is that described in U.S. Pat. No. 6,715,485.

Further examples of formulations have been described above in relation to selegiline.

In the uses of the present invention, the MAO-B inhibitor may advantageously be administered in combination, i.e. sequentially or simultaneously, with another pharmaceutical, where appropriate. The invention envisages products containing an MAO-B inhibitor in combination with one or more such pharmaceuticals, for separate, sequential or simultaneous use in treatment.

### EXAMPLES

### Example 1

### Direct Biochemical Tests to Select ADHD Patients with Low MAO-B Activity

The direct measurement of MAO-B activity can be done in many different ways. Publications describing this include: Wurtman and Axelrod, supra, Jackman et al., supra, Yan et al., 2004, Rapid Comm. Mass. Spectrom., 18(8):834; Harro et al., 2001, Prog. Neuropsychopharmacol. Biol. Psychiatry, 25:1497), Saccone et al., 2002, Alcohol Clin. Exp. Res., 26(5):603, Snell et al. 2002, Alcohol Clin. Exp. Res., 26(7):1105, Ekblom et al. 1998, Neuroscience Lett., 258:101, Shekim et al, 1982, 1984, 1986, supra, Young et al., supra, Hallman et al., supra, Garpenstrand et al., supra, Whitfield et al., supra, Reimherr et al. 1983, psychiatry Res., 11:78 among others.

Below follow a number of examples of assays in vitro in blood platelets, lymphocytes or tissue or in vivo using scanning. The measurement of MAO-B activity may vary from methods described below.

By Radiometry (in Platelets, Method from Harro et al., Supra):

Platelet MAO activity was measured by a radiometric assay with 2-phenylethylamine (β-PEA) as a substrate. Blood samples (7 ml) were drawn in Vacutainer® (Beckton Dickinson, Franklin Lakes, N.J., USA) tubes containing EDTA, and platelet rich plasma was prepared by low speed centrifugation (200 g for 10 min). The platelet concentration was estimated in a Thrombocounter-C® (Coulter Electronics Ltd, Luton, UK) and the platelet rich plasma was stored at −80° C.

At the time of analysis, the plasma samples were thawed and sonicated 4×10s in a Branson Sonifier Cell Disruptor B1® (Branson Sonic Power Company, Danbury, Conn., USA) before estimation of the enzyme activity, which was done as described by Hallman et al., supra. Briefly, the samples were incubated at 37° C. for 4 minutes with [¹⁴C]-β-PEA (50 μM, New England Nuclear, Boston, Mass., USA) and thereafter the reaction was terminated by acidification. The radioactive product formed was extracted by the use of toluene:ethylacetate (1:1, vol/vol) and subsequently quantified in a Packard Tri-Carb Liquid Scintillation Analyzer model 1900 CA® (Packard Instrument Company, Downers Grove, Ill., USA). Enzyme activity can be expressed as nmol/10¹⁰ platelets/min.

By Radiometry (in Tissue Samples, Method from Young et al., Supra):

Tissue was frozen on dry ice within 10 min of the time of resection and was stored at −80° C. Frozen tissue was homogenised for 15 s in 9 volumes of ice-cold 5 mM potassium phosphate buffer (pH 7.5) that contained 0.25 M sucrose. An aliquot of the crude homogenate was diluted and used to measure protein content and MAO activity. The crude homogenate was centrifuged at 650 g for 10 min at 4° C. to remove cellular debris. The supernatant was centrifuged at 10,000 g for 10 min at 4° C. The resulting pellet was resuspended and washed twice in the ice-cold 5 mM potassium phosphate buffer (pH 7.5), diluted, and used to assay protein content and MAO activity.

MAO activity was measured by a modification of Wurtman and Axelrod, supra. The monoamine substrate was PEA. To avoid artefact from mitochondrial isolation or individual differences in mitochondrial number, MAO activity was measured both in the crude homogenates and in the mitochondrial fractions. Linearity of product formation with respect to time of incubation and enzyme concentration was established for the crude homogenate and for the mitochondrial fraction. All assays were performed under conditions well within these linear ranges. “Blanks” were samples in which the homogenates were preincubated at 95° C. for 5 min. The reaction was terminated by the addition of 4N hydrochloric acid. The reaction product was extracted into the organic phase and an aliquot of the organic solvent was added to toluene liquid scintillation fluor. Its radioactivity was measured in a liquid scintillation counter. The assay involved the incubation of 80-fold diluted crude homogenates or 40-fold diluted mitochondrial fractions in the presence of 20 μM [¹⁴C]-β-PEA (12.5 mCi/mmol) for 6 min at 37° C. The reaction product was extracted into toluene. The extraction efficiency was 100%.

Results were expressed as units per milligram of protein or units per gram of tissue weight.

By GC-MS (Method from Zhou et al., (2001) J. Neurol. Neurosurg. Psychiatry 70:229)

Seven milliliters of fasting venous blood was drawn into two plastic tubes containing an anticoagulant agent. Platelet rich plasma for MAO-B measurements was obtained by gentle, centrifugation at 200 g for 10 minutes, and the number of platelets in the platelet rich plasma was determined. Plasma and plasma rich plasma were stored separately at −80° C. until assay. Platelet MAO-B activity was determined by GC-MS (GC-17 A and QP-5000 Mass Spectrometer, Shimadzu, Kyoto, Japan). Sample preparation and the incubation of samples were performed according to the method of Husseini et al. (1995) J. Chromatogr. B., 672:138, with a slight modification. Briefly, the platelet pellet was resuspended with saline to obtain a concentration of 107 platelets/ml and then sonicated for 10 seconds. After preincubation of 50 μl suspension with 80 μl 100 mM KH₂PO₄ at 37° C. for 5 minutes, the suspension was incubated with 20 μM PEA and 0.15 units aldehyde dehydrogenase at 37° C. for 30 minutes. One sample was incubated at 0° C. To another was added 0.24 mM pargyline, an MAO-B inhibitor, as a blank. A capillary column (0.23 mm internal diameter, 30 m long, J and W Scientific Co, Folsom, Calif., USA) coated with DB-5 was used. The mass numbers used for the quantitative analysis were m/z 268, corresponding to phenylacetic acid (PAA), and m/z 282, corresponding to p-methylphenyl acetic acid (mPAA). A peak area measurement was used to estimate the ion current. The calibration curve was highly correlated in standard samples (r=0.947) in the range of 10-500 ng/ml. There was no production of PAA in the samples incubated at 0° C. or with added pargyline. Enzyme activity was calculated from the production of PAA and expressed in pmol product/10⁷ platelets/30 min.

By Spectophotometry (Method from Ivanovic and Majkic-Singh, 1988, J Clin Chem Clin Biochem., 26(7):447)

A simple, continuous spectrophotometric method for the determination of tissue monoamine oxidase based on the oxidation of 2,2′-azino-di-(3-ethylbenzthiazoline-6-sulphonate) (ABTS) using peroxidase has already been described (Ivanovic & Majkic-Singh (1986) Fresenius Z. Anal. Chem. 324, 307). In the present study the method is optimised for platelet monoamine oxidase assay.

By Luminometry (Method from O'Brien et al. (1993) Biochem Pharmacol. 5:46(7):1301)

This method is based on measurement of the light production from the peroxidase-catalysed hemiluminescent oxidation of 5-amino-2,3-dihydro-1,4-phthalazinedione (luminol) by the hydrogen peroxide produced in the MAO reaction. The procedure is suitable for use with a wide range of MAO substrates, although 5-hydroxy-tryptamine, adrenaline and noradrenaline are too readily oxidized by hydrogen peroxide to be used. A particular advantage of this procedure is that it is applicable to the oxidation of substrates which do not yield products, such as an aldehyde or free ammonia, which form the basis of several alternative substrate-independent assay procedures. The application of the procedure to assay the oxidation of benzylamine, tyramine and 2-n-pentylaminoacetamide (milacemide) by a crude mitochondrial preparation from rat liver and purified ox liver MAO-B is demonstrated.

By High-Throughput Fluorescence Assay (Method from Snell et al. 2002, Alcohol Clin. Exp. Res. 26(7):1105)

Platelet MAO-B activity may be assayed by using a high throughput fluorescence assay, such as those described herein.

By High-Performance Liquid Chromatography (Method from Nissinen 1984, J Chromatogr. 309(1):156)

MAO-B activity may be assayed using high performance liquid chromatography techniques such as those described herein.

By the Berthelot Reaction (Method from Uzonov et al. 1978, Acta Physiol Pharmacol Bulg. 4(2):61)

A procedure for monoamine oxidase (MAO) determination with substrate tyramine can be used. The saturation with oxygen and the separation of ammonia from the substrate were omitted. At the end of incubation the samples were deproteinized with ethanol and consecutive centrifugation. The newly-formed ammonia is converted into the coloured compound indophenol, using the procedure of Fenton (1962). The indophenol concentration, respectively NH3 is determined by spectrophotometry at 625 nm, and calculated by comparison with a set of standard amounts of NH3. The enzyme activity is expressed as nanomoles ammonia, formed by 1 mg protein for 1 min.

By Measuring MAO-B Concentrations by Immunoblotting (Method from Snell et al., Supra)

Platelet MAO-B protein concentrations may be measured by analysis of immunoblots probed with a polyclonal antibody selective for MAO, such as those described herein.

By Measuring MAO-B Concentrations by Radiolabelling (Method from Snell et al., Supra)

Quantitative measurements of affinity labelling of platelet MAO may be used by the selective MAO-B catalytic site antagonist [3H]Ro 19-6327, such as those described herein.

By Positron Emission Tomography (Fowler et al. (2003) Proc. Nat. Sciences Soc. 100(20):11600)

A blood sample for plasma cotinine analysis (by gas chromatography; Quest Diagnostics) was taken before the first PET scan, and a breath sample was analysed for carbon monoxide. All twelve subjects completed both scans, and both their hearts and kidneys were visualized in the same scan for all but one of the subjects. Data from eight nonsmokers studied previously was used for comparison (Fowler et al., (2002) J. Nucl. Med. 43:1331).

PET scans comparing L-[¹¹C]deprenyl and L-[¹¹C]deprenyl-D2 [average doses were 6.4±0.9 and 5.6±1.3 mCi (1 Ci=37 GBq), respectively, with specific activity of 250 mCi/μmol at time of injection] were run on a whole-body, Siemens/CTI (Knoxville, Tenn.) HR+ positron emission tomograph (with spatial resolution of ≈4.5-mm full width at half maximum at center of field of view) in 3D dynamic acquisition mode with 2-3 h between scans. Subjects were positioned with a goal of having both the heart and kidneys within the 15-cm axial field of view. Arms were positioned overhead, out of the field of view. Blood sampling and analysis described were used, Fowler et al, supra. Briefly, arterial samples were withdrawn every 2.5 sec for the first 2.5 min by using an automated blood sampling instrument (Ole Dich Instruments, Hvidovre, Denmark), and samples were then hand drawn every minute from 2 to 6 min, and then at 8, 10, 15, 20, 30, 45, and 60 min. Each arterial blood sample was centrifuged, the plasma was pipetted, and the C-11 was counted. Plasma samples at 1, 5, 10, 20, 30, 45, and 60 min were analyzed for L-[¹¹C]deprenyl (or L-[¹¹C]deprenyl-D2) by using a solid-phase extraction method (Alexoff et al. (1995) Nucl. Med. Biol. 22:893). These values were used to correct the arterial plasma time activity curve for the presence of labelled metabolites.

In addition to the dynamic PET scans, we also performed whole-body scans with L-[¹¹C]deprenyl on one of the nonsmokers (7.66 mCi), and one of the smokers (7.42 mCi) who had previously received the dynamic scanning protocol. These whole-body scans were done on a different day than were the dynamic scans, and provided semiquantitative images of all organs, including the brain. PET scanning was initiated 25 min after tracer injection, which is the time when the initial distribution phase of the tracer is complete and when organ accumulation plateaus, reflecting the binding of the tracer to the enzyme. A standard clinical whole-body protocol provided by the PET camera manufacturer was used by using eight bed positions of 10 min each from pelvis to brain. Data were processed by using segmented transmission attenuation correction, and iterative reconstruction and images were scaled so that they could be directly compared.

### Example 2

### Indirect Biochemical Tests to Select ADHD Patients with Low MAO-B Activity

The MAO-B enzyme metabolizes predominantly dopamine (which results in the generation of HVA) and beta-phenylethylamine (which results in the production of PAA). MAO-B activity may be determined by any method that assesses the relative levels of one or more of the substrates and/or one or more of the metabolites. The detection methods below are described for either blood, urine or CSF, though any body fluid sample may be used for detection of these substances.

Urine Concentrations By GC-MS (Method from Zametkin et al. (1985), Arch. Gen. Psychiatry, 42(3):251)

Biochemical analyses for noradrenaline, dopamine and their metabolites MHPG, NMN, VMA, 3,4-dihydroxyphenylacetic acid (DOPAC) and homovanillic acid (HVA) as well as PEA were carried out with gas chromatography and mass spectrometry with the use of deuterated isomers as internal standards. Intra-class correlations were between 0.95 and 0.98 for repeated assay and sensitivity was in the picomole range. After collection, urine was acidified with 6N hydrochloride (3% of the total volume), stored at −80° C. and was analysed in one to three months. Quantification was achieved by comparing the peak heights of the endogeneous non-deuterated compounds with those of the appropriate deuterated internal standards. 24 hour urinary serotonin and 5-HIAA outputs were measured after an extraction procedure with liquid chromatography using electrochemical detection for which within assay coefficient of variation is 4.7% for serotonin and 4.5% for 5-HIAA. The between-assay variation is about 5% for each. Creatinine was assayed using the Here's method (Here, 1950, Proc. Soc. Exp. Biol. Med., 74:148) to assess completeness of urine collection.

HVA Concentrations in Urine Samples (Method from Shekim et al. Biol Psychiatry (1983) 18(6):707-14)

Following the method of Shekim at al, HVA concentrations may be determined from urine samples, such as those described herein.

Urine and Plasma Concentrations of Pea and Related Substances (Method from Baker et al. (1991) Biol. Psychiatry, 29:15)

Levels of urinary PEA (free and total) and or urinary and plasma PAA were analysed by electron-capture gas chromatography as described by Baker et al., supra and Wong et al. (1988) J. Chromatogr., 428(1):140, respectively. Urinary and plasma Phe and P-tyrosine (Tyr) were measured using the procedure of Yeung et al. 1986, J. Chromatogr., 378(2):293. All urinary values were expressed as per g of creatinine in 24-hr samples.

Plasma PEA Concentrations by GC-MS (Method from Zhou et al., Supra)

Seven milliliters of fasting venous blood was drawn into two plastic tubes containing an anticoagulant agent. Platelet rich plasma was centrifuged at 3000 g for 10 minutes for the PEA measurement. Plasma and plasma rich plasma were stored separately at −80° C. until assay. The determination of PEA was performed by GC-MS, as described previously, and expressed in pg/ml (Yamada et al. 1994, Biogenic Amines, 10:295)

Urine Concentrations of Pea & Related Substances by GC-MS-(Method from Zametkin et al., Supra)

After acidification with 3% 6N hydrochloric acid, urine samples were coded and analysed using a gas-chromotographic-mass-fragmentographic method as described elsewhere (Karoum et al. (1979) J. Neurochem., 33(1):201). The compounds assayed include PEA, creatinine, phenylalanine, tyrosine and phenylacetic acid (PAA).

Urine Concentrations of HVA by GC (Method from Shekim et al. J. Child Neurol. (1987) 2(1):50-56)

24-Hr urine collections were obtained and completeness of urine collections was monitored by urinary creatinine levels. Sodium metabisulfite (0.5 g/liter of urine) was added to the urine samples as a preservative for metabolites and the samples were refrigerated immediately after voiding. Aliquots were frozen and later analysed for HVA by the method of Dziedzic et al. (Annal. Biochem. 1972, 47:595).

HVA in Cerebro-Spinal Fluid (CSF) by GC-MS (Method from Reimherr et al, Supra)

Samples of 11 cc of CSF were obtained from women and of 15 cc from men. The samples were collected in calibrated tubes containing 5 mg of ascorbic acid, placed immediately in a dry ice ethanol and acetone bath, and transferred to a −70° C. freezer. Assays for HVA were carried out by gas chromatography mass spectrometry using deuterated internal standards (Godfe et al. 1977, Analytical Chem., 49:917, Gordon et al. 1974, Biological Med., 11:32).

### Example 3

### Genetic Tests to Select ADHD Patients with Genotype and/or Gene Expression Patterns Resulting in Low MAO-B Activity

Genotyping Experiments (from Bengra et al. 2002, Clin Chem. 48 (12):2131-40)

DNA Samples

Genomic DNA was extracted and purified from blood samples and stored at −20° C. before analysis.

Selection of SNPs

Primers for PCR amplification of different SNPs in the genes of interest were designed.

PCR Primers and DNA Amplification

The allele-specific PCR primers and the COM (reverse) primers were designed from published gene sequences using Oligo™ v6.4 primer analysis software (Molecular Biology Insights). PCR primer sequences were synthesized by Midland Certified Reagents. PCR primers contained two allele-specific primers, wild type (WT) and mutant (MUT), and a COM opposite primer per SNP, to amplify each of the SNP loci. The allele-specific primers contain 21-nucleotide (nt) regions (identical to the recognition site of each Universal Amplifluor primer; “tailed”) that are different for one of two labeled primers (green or red). A different sequence tail is then added to the 5′ end of each allele-specific primer. The 21-nt tails on the allele-specific primers are identical with the 21-nt 3′ region of the corresponding Universal Amplifluor (green or red). Final concentrations of PCR reagents were 200 μM of each deoxynucleoside triphosphate, 1.0 U/reaction of either Taq DNA polymerase (Roche Biochemical) or Platinum® Taq DNA polymerase (Life Technologies), 250 nM of both Universal Amplifluor primers and COM (reverse) primer, and 25 nM of both tailed allele-specific primers in 20 μL. The (1×) reaction buffer was 1.8 mM MgCl₂, 50 mM KCl, and 10 mM Tris, pH 8.30. The Amplifluor reagent system (Serologicals Corp.) includes two Universal Amplifluor primers [labeled with fluorescein (FAM) or sulforhodamine (SR)], 10×PCR buffer, and deoxynucleoside triphosphates]. PCR reactions were set up and performed in optically clear PCR microplates (VWR Scientific Products) and sealed with PCR plate-sealer adhesive tape (Robbins Scientific Corp.).

Amplifications were performed in an PTC-200 gradient thermal cycler (M J Research) with the following conditions: a pseudo-hot start of 5-10 s at 94° C., denaturation of 4 min at 95° C., then 35 cycles (10 s at 94° C., 20 s at 55° C., and 40 s at 72° C.), followed by 3 min of final extension at 72° C. PCR reactions were held at 20° C. until fluorescence measurements could be performed. SNP PCR reactions were optimized by performing PCRs with several 10×PCR buffers [Buffer K: 600 mM Tris-HCl (pH 9.5), 150 mM (NH₄)₂SO₄, and 25 mM MgCl₂; Buffer N: 600 mM Tris-HCl (pH 10.0), 150 mM (NH₄)₂SO₄, and 20 mM MgCl₂; and Buffer I: 100 mM Tris-HCl (pH 8.3), 500 mM KCl, and 18 mM MgCl₂] and then analyzing the PCR products by gel electrophoresis for yield and specificity. One buffer that gave maximum amplicon yield and specificity was subsequently selected for all SNP PCRs. We also performed temperature-gradient PCRs to investigate the potential influence of Amplifluors on amplicon yield and specificity of PCR as a function of annealing temperature (range, 50-70° C.). The optimum combination of target amount and cycle number that provided the best yield of PCR amplicon was also determined.

Fluorescent Measurements and Data Analysis

Total fluorescence (as relative fluorescence units) of labeled Universal Amplifluor primer-containing amplicons was quantified through the top of each well of open PCR microplates using a Victor™ 1420 fluorescence microplate reader (Perkin-Elmer Wallac, Inc.). The microplate reader was equipped with the narrow bandpass filters to quantify FAM (excitation, 485 nm; emission, 535 nm) and SR (excitation, 585 nm; emission, 620 nm). Fluorescence results were transferred to separate Excel worksheets for analysis, and scatterplots for each SNP locus were built as follows. Signals from WT (usually FAM-labeled primer) alleles were plotted along the x axes, whereas signals from MUT (usually SR-labeled primer) alleles were plotted along the y axes. In the typical labeling scheme, fluorescence of samples that have a homozygous WT genotype accumulate along the x axis, whereas signals from samples with the homozygous MUT genotype accumulate along the y axis. Signals from the heterozygous genotypes tend to cluster along a diagonal line between the x and y axes. Signals of no-template (blank) PCRs appear near the x,y origin.

Genotype frequencies were compared with Hardy-Weinberg expectations, and allele frequencies were compared between normotensive and hypertensive groups by the method of Roussett and Raymond (1995, Genetics 140(4):1413-9).

Sequence Confirmation of SNP Amplicons

Confirmation of the WT and MUT amplicon sequences at five of six SNP loci was performed by use of a sequence-appropriate restriction endonuclease to digest the PCR products. After fluorescence quantification, PCR amplicons were typically purified by precipitation using 2× volumes of absolute ethanol and then resuspended with deionized IH₂O and restricted with one to two units of an appropriate restriction endonuclease. After incubation, reaction mixtures (volume, 20 μL) were separated by gel electrophoresis on 4% agarose gels in 1× Tris-acetate-EDTA buffer, followed by staining with ethidium bromide. Sizes of digested amplicons were determined by comparison with a 10-bp size ladder (New England Biolabs).

Similarly gene expression tests measuring the level of MAO-B messenger RNA expression may be measured in any human tissue samples as exemplified below. These technologies could include RT-PCR related methods such as by microarray, or by the ABI-Taqman™ technology (see WO-00/05409). Tissues which may be used include lymphocytes (see Gladkevich et al., 2004, Prog Neuropsychopharmacol Biol Psychiatry. 28(3):559-76).

Expression of Messenger RNA

Expression of messenger RNA may be performed using the methods described in, for example, WO 00/05409.

RNA Extraction

Total RNA will be extracted from the tissues using Trizol according to the manufacturer's protocol. The RNA will only be used for cDNA synthesis if the optical absorbance ratio (A260/A280) >1.8 and it has intact 18 and 28S ribosomal RNA.

Primer/Probe Design

Primers and TaqMan probes are designed to amplify specific GenBank sequences. These primers and probes are then homology searched against GenBank to confirm that they are specific for the targets from which they were designed. PCR reactions for the target gene are duplexed, with glyceraldehyde-3-phosphate dehydrogenase (GAPDH), which is used as a marker of intact RNA. The target probe is labelled with the fluor 6-FAM whilst the probe for GAPDH is labelled with the fluor VIC. Primers and probes will be designed across exon/exon boundaries or where this is not possible the samples will be DNase I treated. This is to avoid any amplification from genomic DNA, which has been co isolated with the total RNA.

cDNA Synthesis

This will be synthesised from 50 ng of total RNA from each of the tissues being studied. The cDNA will synthesised using random primers, using a high capacity cDNA archive kit (Applied Biosystems 4322171).

The cDNA derived from the 50 ng total RNA for each sample will be subjected to PCR amplification in a single reaction to identify both target and GAPDH transcripts. Primers and probes for the target and GAPDH genes will be added to the reaction mix along with the appropriate buffer, nucleotides and Taq polymerase. The PCR conditions will be: 95° C. for 10 minutes, followed by 45 cycles of 95° C. for 15 seconds and 60° C. for 45 seconds. PCR amplification curves will be analysed to yield Ct values and these values will be used to determine the starting mRNA copy number of both target and GAPDH genes by extrapolation from standard curves generated from known amounts of PCR product for both the target and GAPDH.

1. A method for determining whether an ADHD patient is suitable for treatment with a monoamine oxidase type B (MAO-B) inhibitor which comprises:
(a) determining ex vivo the level of MAO-B activity in an ADHD patient;
(b) if said activity of MAO-B falls within the 30% lower percentile of the full range of MAO-B activity within a normal population, testing said patient for symptoms of ADHD predominantly inattentive type (in accordance with DSM IV); and
(c) if said activity of MAO-B falls within the 30% lower percentile of the full range of MAO-B activity within a normal population and said patient tests for symptoms of ADHD predominantly inattentive type, concluding that said patient is suitable for treatment with an MAO-B inhibitor.

2. A method for determining whether an ADHD patient is suitable for treatment with an MAO-B inhibitor which comprises:
(a) selecting patients diagnosed with ADHD predominantly inattentive type in accordance with DSM IV;
(b) testing ex vivo the patients selected according to step (a) for activity of MAO-B; and
(c) submitting patients with an MAO-B level within the 30% lower percentile of the full range of MAO-B activity within a normal population for treatment with an MAO-B inhibitor.

3. A method according to claim 1 wherein the 30% lower percentile of the full range of MAO-B activity within a normal population is defined as less than or equal to 30 nmol/ml/h/number of platelets×10⁻⁶; or less than or equal to 8 nmol/min/10¹⁰ platelets; or less than or equal to 3 MAO-B units/10⁸ platelets.

4. A method according to claim 1 which comprises determining MAO-B activity in a blood, platelet, brain biopsy, cerebrospinal fluid (CSF), lymphocyte or liver sample.

5. A method according to claim 1 which comprises determining MAO-B activity by radiometry, gas chromatography, mass spectrometry or a genetic test.

6. A method according to claim 5 wherein the genetic test comprises the use of one or more probes or primers for detecting variable number tandem repeats (VNTRs) and/or single nucleotide polymorphisms (SNPs).

7. A method according to claim 6 which comprises detection of one or more VNTRs selected from GCTGCCAAGAAGAAGGTG (SEQ ID NO:1), TGGATGGATGAA (SEQ ID NO:3), ACCATCATC, CACACACATG (SEQ ID NO:5), ATTTATTAACT (SEQ ID NO:7), TGTATCAGCCATTTCCAAC (SEQ ID NO:9), TTTTACAAAGTAATATTTG (SEQ ID NO:11), ATTTGTTTTACAAATTTTTACAAAGTA (SEQ ID NO:13), ATAGATAT, TTCAAAGCAAATGTTGAG (SEQ ID NO:15), TGTTTATGAAACAAA (SEQ ID NO:17), GATTTCATTCATAAGATACAC (SEQ ID NO:19) and CTTGCTCAGTTACAAGA (SEQ ID NO:21).

8. A method according to claim 5 wherein the genetic test comprises:
(a) determining the presence of a G-allele in the MAO-B intron 13;
(b) assessing the genotype at the AP-2beta gene; or
(c) assessing the genotype at or expression levels of the c-Jun, Egr-1 and Sp1 genes.

9. A method according to claim 1 which further comprises treating the patient with an MAO-B inhibitor.

10. (canceled)

11. A method according to claim 9 wherein the MAO-B inhibitor is selected from selegiline, rasagiline, safinamide, mofegiline and lazabemide, SL-25.118, milacemide, LU-53439, SL-34.0026, EXP-631, M-2-PP, SL-25.1131, FA-87, RS-1636, NW-1048, himantane, excitatory amino acids, FA-73, ladostigil, CHF-3381, selegiline analogs, befloxatone, AIT-203 and AIT-297.

12-15. (canceled)

16. A method according to claim 9 wherein the MAO-B inhibitor is a selective inhibitor.

17. (canceled)

18. A nucleic acid probe or primer for detection of a VNTR motif, comprising:
(a) a fragment of the flanking sequence of the VNTR motif; or
(b) a nucleic acid sequence complementary to (a); wherein the VNTR motif is selected from GCTGCCAAGAAGAAGGTG (SEQ ID NO:1), TGGATGGATGAA (SEQ ID NO:3), ACCATCATC, CACACACATG (SEQ ID NO:5), ATTTATTAACT (SEQ ID NO:7), TGTATCAGCCATTTCCAAC (SEQ ID NO:9), TTTTACAAAGTAATATTTG (SEQ ID NO:11), ATTTGTTTTACAAATTTTTACAAAGTA (SEQ ID NO:13), ATAGATAT, TTCAAAGCAAATGTTGAG (SEQ ID NO:15), TGTTTATGAAACAAA (SEQ ID NO:17), GATTTCATTCATAAGATACAC (SEQ ID NO:19) and CTTGCTCAGTTACAAGA (SEQ ID NO:21) and wherein the flanking sequence is the sequence from 1 to 250 bases upstream or downstream of the motif.

19. A probe or primer generated according to claim 36 for use in medicine.

20. (canceled)

21. A kit for use in diagnosing and/or treating ADHD in a subject which kit comprises a probe or primer generated according to claim 36.

22. A method according to claim 2 wherein the 30% lower percentile of the full range of MAO-B activity within a normal population is defined as less than or equal to 30 nmol/ml/h/number of platelets×10⁻⁶; or less than or equal to 8 nmol/min/10¹⁰ platelets; or less than or equal to 3 MAO-B units/10⁸ platelets.

23. A method according to claim 2 which comprises determining MAO-B activity in a blood, platelet, brain biopsy, cerebrospinal fluid (CSF), lymphocyte or liver sample.

24. A method according to claim 2 which comprises determining MAO-B activity by radiometry, gas chromatography, mass spectrometry or a genetic test.

25. A method according to claim 2 wherein the genetic test comprises the use of one or more probes or primers for detecting variable number tandem repeats (VNTRs) and/or single nucleotide polymorphisms (SNPs).

26. A method according to claim 24 which comprises detection of one or more VNTRs selected from GCTGCCAAGAAGAAGGTG (SEQ ID NO:1), TGGATGGATGAA (SEQ ID NO:3), ACCATCATC, CACACACATG (SEQ ID NO:5), ATTTATTAACT (SEQ ID NO:7), TGTATCAGCCATTTCCAAC (SEQ ID NO:9), TTTTACAAAGTAATATTTG (SEQ ID NO:11), ATTTGTTTTACAAATTTTTACAAAGTA (SEQ ID NO:13), ATAGATAT, TTCAAAGCAAATGTTGAG (SEQ ID NO:15), TGTTTATGAAACAAA (SEQ ID NO:17), GATTTCATTCATAAGATACAC (SEQ ID NO:19) and CTTGCTCAGTTACAAGA (SEQ ID NO:21).

27. A method according to claim 24 wherein the genetic test comprises:
(a) determining the presence of a G-allele in the MAO-B intron 13;
(b) assessing the genotype at the AP-2beta gene; or
(c) assessing the genotype at or expression levels of the c-Jun, Egr-1 and Sp1 genes.

28. A method according to claim 2 which further comprises treating the patient with an MAO-B inhibitor.

29. A method of treating a patient with ADHD comprising administering an MAO-B inhibitor to the patient, wherein the suitability of the patient for such treatment has been determined by:
(a) determining ex vivo the level of MAO-B activity in the ADHD patient;
(b) if said activity of MAO-B is within the 30% lower percentile of the full range of MAO-B activity with a normal population, testing said patient for symptoms of ADHD predominantly inattentive type (in accordance with DSM IV); and
(c) if said activity of MAO-B falls within the 30% lower percentile of the full range of MAO-B activity within a normal population and said patient tests for symptoms of ADHD predominantly inattentive type, concluding that said patient is suitable for treatment with a monoamine oxidase type B inhibitor.

30. A method according to claim 29 wherein the MAO-B inhibitor is selected from selegiline, rasagiline, safinamide, mofegiline, and lazabemide, SL-25.118, milacemide, LU-53439, SL-34.0026, EXP-631, M-2-PP, SL-25.1131, FA-87, RS-1636, NW-1048, himantane, excitatory amino acids, FA-73, ladostigil, CHF-3381, selegiline analogs, befloxatone, AIT-203 and AIT-297.

31. A method according to claim 29 wherein the MAO-B inhibitor is a selective inhibitor.

32. A method of treating a patient with ADHD, comprising administering an MAO-B inhibitor to the patient wherein the MAO-B inhibitor is other than selegiline, pargiline or rasagiline.

33. A method according to claim 32 wherein the patient is of the ADHD predominantly inattentive subtype and/or has low MAO-B activity.

34. A method according to claim 32 wherein the MAO-B inhibitor is selected from SL-25.118, lazabemide, safinamide, mofegiline, milacemide, LU-53439, SL-34.0026, EXP-631, M-2-PP, SL-25.1131, FA-87, RS-1636, NW-1048, himantane, excitatory amino acids, FA-73, ladostigil, CHF-3381, selegiline analogs, befloxatone, AIT-203 and AIT-297.

35. A method according to claim 32 wherein the MAO-B inhibitor is a selective inhibitor.

36. A method for generating a nucleic acid probe or primer for the assessment of a genotype predictive of MAO-B activity in an ADHD patient, comprising designing the probe or primer based on the flanking sequence of a VNTR sequence motif, wherein the VNTR sequence motif is selected from GCTGCCAAGAAGAAGGTG (SEQ ID NO:1), TGGATGGATGAA (SEQ ID NO:3), ACCATCATC, CACACACATG (SEQ ID NO:5), ATTTATTAACT (SEQ ID NO:7), TGTATCAGCCATTTCCAAC (SEQ ID NO:9), TTTTACAAAGTAATATTTG (SEQ ID NO:11), ATTTGTTTTACAAATTTTTACAAAGTA (SEQ ID NO:13), ATAGATAT, TTCAAAGCAAATGTTGAG (SEQ ID NO:15), TGTTTATGAAACAAA (SEQ ID NO:17), GATTTCATTCATAAGATACAC (SEQ ID NO:19) and CTTGCTCAGTTACAAGA (SEQ ID NO:21), and wherein the flanking sequence is the sequence from 1 to 250 bases upstream or downstream of the motif.

37. A method for in vitro determination of genotype predictive of MAO-B activity comprising contacting a probe or primer according to claim 18 with a sample taken from the patient.
